SEQUENCE LISTING (109 sequences)

SEQ ID NO: 1 - protein, 621 aa, Homo sapiens [sequence omitted]

SEQ ID NO: 2 - DNA, 1866 bp, Homo sapiens
  source 1..1866: mol_type = genomic DNA
atggccttgccaacggctcgacccctgttggggtcctgtgggacccccgccctcggcagcctcctgttcctgctcttcagcctcggatgggtgcagccctcgaggaccctggctggagagacagggcaggctgcacccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagaggccctctcggggacgccctgcctcctaggacctggacctgttctcaccgtcctggcactgctcctagcctccaccctggcctga

SEQ ID NO: 3 - DNA, 22 bp, synthetic construct
  source 1..22: mol_type = other DNA
cctggtgcatgatgggaagttc

SEQ ID NO: 4 - DNA, 27 bp, synthetic construct
  source 1..27: mol_type = other DNA
  misc_feature 1..24: note = DNA
  misc_feature 25..27: note = RNA
gtcgcacggtccatcgcagcagtcggg

SEQ ID NO: 5 - DNA, 20 bp, synthetic construct
  source 1..20: mol_type = other DNA
ccagctgaactggacctcgg

SEQ ID NO: 6 - DNA, 24 bp, synthetic construct
  source 1..24: mol_type = other DNA
gtcgcacggtccatcgcagcagtc

SEQ ID NO: 7 - DNA, 49 bp, synthetic construct
  source 1..49: mol_type = other DNA
ttcccttcaattcaagtaacaggacaagttcagcttgtagagtcaggcg

SEQ ID NO: 8 - DNA, 45 bp, synthetic construct
  source 1..45: mol_type = other DNA
ttcccttcaattcaagtaacaggacaggttcagctggtggagtcc

SEQ ID NO: 9 - DNA, 49 bp, synthetic construct
  source 1..49: mol_type = other DNA
ttcccttcaattcaagtaacaggacaagttcagcttgtcgagagtggtg

SEQ ID NO: 10 - DNA, 48 bp, synthetic construct
  source 1..48: mol_type = other DNA
ttcccttcaattcaagtaacaggacaggtccaattggtggaatctggg

SEQ ID NO: 11 - DNA, 46 bp, synthetic construct
  source 1..46: mol_type = other DNA
ttcccttcaattcaagtaacaggacaagtgcaacttgtcgagagcg

SEQ ID NO: 12 - DNA, 50 bp, synthetic construct
  source 1..50: mol_type = other DNA
ttcccttcaattcaagtaacaggagacgtacaacttgtggaatcaggtgg

SEQ ID NO: 13 - DNA, 48 bp, synthetic construct
  source 1..48: mol_type = other DNA
ttcccttcaattcaagtaacaggacaggttcaactcgttgagtcaggc

SEQ ID NO: 14 - DNA, 48 bp, synthetic construct
  source 1..48: mol_type = other DNA
ttcccttcaattcaagtaacaggacaagttcagttggtggaaagtggg

SEQ ID NO: 15 - DNA, 53 bp, synthetic construct
  source 1..53: mol_type = other DNA
ttcccttcaattcaagtaacaggacaagtaaaacttgaagaatcaggtggtgg

SEQ ID NO: 16 - DNA, 46 bp, synthetic construct
  source 1..46: mol_type = other DNA
ttcccttcaattcaagtaacaggacaagtccagctcgtcgagagtg

SEQ ID NO: 17 - DNA, 46 bp, synthetic construct
  source 1..46: mol_type = other DNA
ttcccttcaattcaagtaacaggacaggtccaattggtggaaagcg

SEQ ID NO: 18 - DNA, 47 bp, synthetic construct
  source 1..47: mol_type = other DNA
ttcccttcaattcaagtaacaggagaggtccaggtcgtcgaatcagg

SEQ ID NO: 19 - DNA, 49 bp, synthetic construct
  source 1..49: mol_type = other DNA
ttcccttcaattcaagtaacaggagaggtacaacttgtggaaagcggtg

SEQ ID NO: 20 - DNA, 46 bp, synthetic construct
  source 1..46: mol_type = other DNA
ttcccttcaattcaagtaacaggagaagtgcaggttgtcgagtccg

SEQ ID NO: 21 - DNA, 48 bp, synthetic construct
  source 1..48: mol_type = other DNA
ttcccttcaattcaagtaacaggacaagtaaaactcgaagagagcggg

SEQ ID NO: 22 - DNA, 50 bp, synthetic construct
  source 1..50: mol_type = other DNA
ttcccttcaattcaagtaacaggacagcttcaactggtagagagtggtgg

SEQ ID NO: 23 - DNA, 48 bp, synthetic construct
  source 1..48: mol_type = other DNA
ttcccttcaattcaagtaacaggacaggtccaactggtagaaagtggg

SEQ ID NO: 24 - DNA, 47 bp, synthetic construct
  source 1..47: mol_type = other DNA
ttcccttcaattcaagtaacaggacaggtccaactggtagagtcagg

SEQ ID NO: 25 - DNA, 47 bp, synthetic construct
  source 1..47: mol_type = other DNA
ttcccttcaattcaagtaacaggagaggtccaactcgtcgaatctgg

SEQ ID NO: 26 - DNA, 50 bp, synthetic construct
  source 1..50: mol_type = other DNA
ttcccttcaattcaagtaacaggacaagtgcaattggttgaatctggtgg

SEQ ID NO: 27 - DNA, 48 bp, synthetic construct
  source 1..48: mol_type = other DNA
ttcccttcaattcaagtaacaggagaagtccaacttgtagagagtggg

SEQ ID NO: 28 - DNA, 45 bp, synthetic construct
  source 1..45: mol_type = other DNA
ttcccttcaattcaagtaacaggacaggtgcaggttgtggaaagc

SEQ ID NO: 29 - DNA, 49 bp, synthetic construct
  source 1..49: mol_type = other DNA
ttcccttcaattcaagtaacaggacaggtgcaattggtagagtctggag

SEQ ID NO: 30 - DNA, 46 bp, synthetic construct
  source 1..46: mol_type = other DNA
ttcccttcaattcaagtaacaggacaagtgcagttggttgagagcg

SEQ ID NO: 31 - DNA, 47 bp, synthetic construct
  source 1..47: mol_type = other DNA
ttcccttcaattcaagtaacaggacaggttcaactcgtcgaatccgg

SEQ ID NO: 32 - DNA, 46 bp, synthetic construct
  source 1..46: mol_type = other DNA
ttcccttcaattcaagtaacaggacaagttcagcttgtggagagcg

SEQ ID NO: 33 - DNA, 49 bp, synthetic construct
  source 1..49: mol_type = other DNA
ttcccttcaattcaagtaacaggagaagttcagcttgttgaatcaggcg

SEQ ID NO: 34 - DNA, 50 bp, synthetic construct
  source 1..50: mol_type = other DNA
ttcccttcaattcaagtaacaggacaagttcaactcgtagaatctggcgg

SEQ ID NO: 35 - DNA, 50 bp, synthetic construct
  source 1..50: mol_type = other DNA
ttcccttcaattcaagtaacaggagaagttcaacttgtcgaaagcggagg

SEQ ID NO: 36 - DNA, 46 bp, synthetic construct
  source 1..46: mol_type = other DNA
ttcccttcaattcaagtaacaggacaggtccagcttgtcgaatccg

SEQ ID NO: 37 - DNA, 47 bp, synthetic construct
  source 1..47: mol_type = other DNA
ctcgcaatagttggcgctggcgtcggcgaggagacggtgaccgtggt

SEQ ID NO: 38 - DNA, 47 bp, synthetic construct
  source 1..47: mol_type = other DNA
ctcgcaatagttggcgctggcgtcggcgaggagactgtgagagtggt

SEQ ID NO: 39 - DNA, 47 bp, synthetic construct
  source 1..47: mol_type = other DNA
ctcgcaatagttggcgctggcgtcggcgcagagacagtgaccagagt

SEQ ID NO: 40 - DNA, 47 bp, synthetic construct
  source 1..47: mol_type = other DNA
ctcgcaatagttggcgctggcgtcggcgaggagacggtgactgaggt

SEQ ID NO: 41 - DNA, 80 bp, synthetic construct
  source 1..80: mol_type = other DNA
  modified_base 76: mod_base = OTHER; note = Nucleotide with phosphorothioate linkage
  modified_base 77: mod_base = OTHER; note = Nucleotide with phosphorothioate linkage
  modified_base 78: mod_base = OTHER; note = Nucleotide with phosphorothioate linkage
  modified_base 79: mod_base = OTHER; note = Nucleotide with phosphorothioate linkage
aatgatacggcgaccaccgagatctacactctttccctacacgacgctcttccgatctccatgctcaggctgctcttggc

SEQ ID NO: 42 - DNA, 81 bp, synthetic construct
  source 1..81: mol_type = other DNA
  modified_base 77: mod_base = OTHER; note = Nucleotide with phosphorothioate linkage
  modified_base 78: mod_base = OTHER; note = Nucleotide with phosphorothioate linkage
  modified_base 79: mod_base = OTHER; note = Nucleotide with phosphorothioate linkage
  modified_base 80: mod_base = OTHER; note = Nucleotide with phosphorothioate linkage
aatgatacggcgaccaccgagatctacactctttccctacacgacgctcttccgatctcccatgctcaggctgctcttggc

SEQ ID NO: 43 - DNA, 82 bp, synthetic construct
  source 1..82: mol_type = other DNA
  modified_base 78: mod_base = OTHER; note = Nucleotide with phosphorothioate linkage
  modified_base 79: mod_base = OTHER; note = Nucleotide with phosphorothioate linkage
  modified_base 80: mod_base = OTHER; note = Nucleotide with phosphorothioate linkage
  modified_base 81: mod_base = OTHER; note = Nucleotide with phosphorothioate linkage
aatgatacggcgaccaccgagatctacactctttccctacacgacgctcttccgatctgcccatgctcaggctgctcttggc

SEQ ID NO: 44 - DNA, 83 bp, synthetic construct
  source 1..83: mol_type = other DNA
  modified_base 79: mod_base = OTHER; note = Nucleotide with phosphorothioate linkage
  modified_base 80: mod_base = OTHER; note = Nucleotide with phosphorothioate linkage
  modified_base 81: mod_base = OTHER; note = Nucleotide with phosphorothioate linkage
  modified_base 82: mod_base = OTHER; note = Nucleotide with phosphorothioate linkage
aatgatacggcgaccaccgagatctacactctttccctacacgacgctcttccgatctagcccatgctcaggctgctcttggc

SEQ ID NO: 45 - DNA, 84 bp, synthetic construct
  source 1..84: mol_type = other DNA
  modified_base 80: mod_base = OTHER; note = Nucleotide with phosphorothioate linkage
  modified_base 81: mod_base = OTHER; note = Nucleotide with phosphorothioate linkage
  modified_base 82: mod_base = OTHER; note = Nucleotide with phosphorothioate linkage
  modified_base 83: mod_base = OTHER; note = Nucleotide with phosphorothioate linkage
aatgatacggcgaccaccgagatctacactctttccctacacgacgctcttccgatctcaacccatgctcaggctgctcttggc

SEQ ID NO: 46 - DNA, 86 bp, synthetic construct
  source 1..86: mol_type = other DNA
  modified_base 82: mod_base = OTHER; note = Nucleotide with phosphorothioate linkage
  modified_base 83: mod_base = OTHER; note = Nucleotide with phosphorothioate linkage
  modified_base 84: mod_base = OTHER; note = Nucleotide with phosphorothioate linkage
  modified_base 85: mod_base = OTHER; note = Nucleotide with phosphorothioate linkage
aatgatacggcgaccaccgagatctacactctttccctacacgacgctcttccgatcttgcaccccatgctcaggctgctcttggc

SEQ ID NO: 47 - DNA, 87 bp, synthetic construct
  source 1..87: mol_type = other DNA
  modified_base 83: mod_base = OTHER; note = Nucleotide with phosphorothioate linkage
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
aatgatacggcgaccaccgagatctacactctttccctacacgacgctcttccgatctacgcaacccatgctcaggctgctcttggc

SEQ ID NO: 48 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
aatgatacggcgaccaccgagatctacactctttccctacacgacgctcttccgatctgaagacccccatgctcaggctgctcttggc

SEQ ID NO: 49 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagataaccgcgggtgactggagttcagacgtgtgctcttccgatctcaatagttggcgctggcgtcgg

SEQ ID NO: 50 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatggttataagtgactggagttcagacgtgtgctcttccgatctcaatagttggcgctggcgtcgg

SEQ ID NO: 51 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatccaagtccgtgactggagttcagacgtgtgctcttccgatctcaatagttggcgctggcgtcgg

SEQ ID NO: 52 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatttggacttgtgactggagttcagacgtgtgctcttccgatctcaatagttggcgctggcgtcgg

SEQ ID NO: 53 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatcagtggatgtgactggagttcagacgtgtgctcttccgatctcaatagttggcgctggcgtcgg

SEQ ID NO: 54 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagattgacaagcgtgactggagttcagacgtgtgctcttccgatctcaatagttggcgctggcgtcgg

SEQ ID NO: 55 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatctagcttggtgactggagttcagacgtgtgctcttccgatctcaatagttggcgctggcgtcgg

SEQ ID NO: 56 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagattcgatccagtgactggagttcagacgtgtgctcttccgatctcaatagttggcgctggcgtcgg

SEQ ID NO: 57 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatcctgaactgtgactggagttcagacgtgtgctcttccgatctcaatagttggcgctggcgtcgg

SEQ ID NO: 58 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatttcaggtcgtgactggagttcagacgtgtgctcttccgatctcaatagttggcgctggcgtcgg

SEQ ID NO: 59 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatagtagagagtgactggagttcagacgtgtgctcttccgatctcaatagttggcgctggcgtcgg

SEQ ID NO: 60 - DNA, 88 bp, synthetic construct
  source 1..88: mol_type = other DNA
  modified_base 84: mod_base = OTHER; note = Nucleotide with phosphorothioate linkage
  modified_base 85: mod_base = OTHER; note = Nucleotide with phosphorothioate linkage
  modified_base 86: mod_base = OTHER; note = Nucleotide with phosphorothioate linkage
  modified_base 87: mod_base = OTHER; note = Nucleotide with phosphorothioate linkage
caagcagaagacggcatacgagatgacgagaggtgactggagttcagacgtgtgctcttccgatctcaatagttggcgctggcgtcgg

SEQ ID NO: 61 - DNA, 8035 bp, synthetic construct
  source 1..8035: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccccgggtttattacagggacagcagagatccactttggcgccggctcgagttttaaaagaaaaggggggattggggggtacagtgcaggggaaagaatagtagacataatagcaacagacatacaaactaaagaattacaaaaacaaattacaaaaattcaaaatttttcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgctcaggctgctcttggctctcaacttattcccttcaattcaagtaacaggagggtcttccttttttgaagacccgacgccagcgccaactattgcgagtcagcctctcagtctgcgacctgaggcttgtcgaccagcagccggaggcgcagtgcacacgagggggctggacttcgcctgtgatccttctaagcccttttgggtgctggtggtggttggtggagtcctggcttgctatagcttgctagtaacagtggcctttattattttctgggtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagcgccacgaacttctctctgttaaagcaagcaggcgacgtggaagaaaaccccggtcccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctttgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt

SEQ ID NO: 62 - protein, 511 aa, synthetic construct
MLRLLLALNLFPSIQVTGGSSFFEDPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRASATNFSLLKQAGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 63 - DNA, 7521 bp, synthetic construct
  source 1..7521: mol_type = other DNA
ggccgccagcacagtggtcgatcgacgataaaataaaagattttatttagtctccagaaaaaggggggaatgaaagaccccacctgtaggtttggcaagctagcttaagtaacgccattttgcaaggcatggaaaaatacataactgagaatagaaaagttcagatcaaggtcaggaacagatggaacagggtcgcgtcccgcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccagctttgctcttaggagtttcctaatacatcccaaactcaaatatataaagcatttgacttgttctatgccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccaagctggaggaaaaactgtttcatacagaaggcgtggaggaaaaactgtttcatacagaaggcgtggaggaaaaactgtttcatacagaaggcgtggaggaaaaactgtttcatacagaaggcgtcgcgaattcgcggagactctagagggtatataatggaagctcgatttccagcttggcattccggtactgttggtaaacaccaagctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtttcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgctagccgggccaagcggtccggatccggacagtgcaccaactatgcgctgctgaaactggcgggcgatgtggaaagcaacccgggcccgatggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaaccgcgccaagcgctcgggttcgggtgccaccaacttcagcctgctgaagcaggccggcgacgtggaggagaaccccggccccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagataa

SEQ ID NO: 64 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 65 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 66 - DNA, 199 bp, synthetic construct
  source 1..199: mol_type = other DNA
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcag

SEQ ID NO: 67 - DNA, 21 bp, synthetic construct
  source 1..21: mol_type = other DNA
cgcaaatgggcggtaggcgtg

SEQ ID NO: 68 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 69 - DNA, 126 bp, synthetic construct
  source 1..126: mol_type = other DNA
ctctctcgacgcaggactcggcttgctgaagcgcgcacggcaagaggcgaggggcggcgactggtgagtacgccaaaaattttgactagcggaggctagaaggagagagatgggtgcgagagcgtc

SEQ ID NO: 70 - DNA, 234 bp, synthetic construct
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 71 - DNA, 118 bp, synthetic construct
  source 1..118: mol_type = other DNA
ttttaaaagaaaaggggggattggggggtacagtgcaggggaaagaatagtagacataatagcaacagacatacaaactaaagaattacaaaaacaaattacaaaaattcaaaatttt

SEQ ID NO: 72 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 73 - DNA, 762 bp, synthetic construct
  source 1..762: mol_type = other DNA
atgctcaggctgctcttggctctcaacttattcccttcaattcaagtaacaggagggtcttccttttttgaagacccgacgccagcgccaactattgcgagtcagcctctcagtctgcgacctgaggcttgtcgaccagcagccggaggcgcagtgcacacgagggggctggacttcgcctgtgatccttctaagcccttttgggtgctggtggtggttggtggagtcctggcttgctatagcttgctagtaacagtggcctttattattttctgggtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagc

SEQ ID NO: 74 - DNA, 51 bp, synthetic construct
  source 1..51: mol_type = other DNA
atgctcaggctgctcttggctctcaacttattcccttcaattcaagtaaca

SEQ ID NO: 75 - DNA, 111 bp, synthetic construct
  source 1..111: mol_type = other DNA
ccgacgccagcgccaactattgcgagtcagcctctcagtctgcgacctgaggcttgtcgaccagcagccggaggcgcagtgcacacgagggggctggacttcgcctgtgat

SEQ ID NO: 76 - DNA, 86 bp, synthetic construct
  source 1..86: mol_type = other DNA
ctaagcccttttgggtgctggtggtggttggtggagtcctggcttgctatagcttgctagtaacagtggcctttattattttctgg

SEQ ID NO: 77 - DNA, 132 bp, synthetic construct
  source 1..132: mol_type = other DNA
gtgaggaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 78 - DNA, 342 bp, synthetic construct
  source 1..342: mol_type = other DNA
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 79 - DNA, 57 bp, synthetic construct
  source 1..57: mol_type = other DNA
gccacgaacttctctctgttaaagcaagcaggcgacgtggaagaaaaccccggtccc

SEQ ID NO: 80 - DNA, 717 bp, synthetic construct
  source 1..717: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 81 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctttgccttcgccctcagacgagtcggatctccctttgggccgcctccccgc

SEQ ID NO: 82 - DNA, 12 bp, synthetic construct
  source 1..12: mol_type = other DNA
tcggccctcaat

SEQ ID NO: 83 - DNA, 234 bp, synthetic construct
  source 1..234: mol_type = other DNA
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 84 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 85 - DNA, 358 bp, synthetic construct
  source 1..358: mol_type = other DNA
aagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctg

SEQ ID NO: 86 - DNA, 417 bp, synthetic construct
  source 1..417: mol_type = other DNA
ggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcc

SEQ ID NO: 87 - DNA, 375 bp, synthetic construct
  source 1..375: mol_type = other DNA
ccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 88 - DNA, 603 bp, synthetic construct
  source 1..603: mol_type = other DNA
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtttcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgctagc

SEQ ID NO: 89 - DNA, 12 bp, synthetic construct
  source 1..12: mol_type = other DNA
cgggccaagcgg

SEQ ID NO: 90 - DNA, 12 bp, synthetic construct
  source 1..12: mol_type = other DNA
tccggatccgga

SEQ ID NO: 91 - DNA, 12 bp, synthetic construct
  source 1..12: mol_type = other DNA
tcgggttcgggt

SEQ ID NO: 92 - DNA, 60 bp, synthetic construct
  source 1..60: mol_type = other DNA
cagtgcaccaactatgcgctgctgaaactggcgggcgatgtggaaagcaacccgggcccg

SEQ ID NO: 93 - DNA, 1128 bp, synthetic construct
  source 1..1128: mol_type = other DNA
atggcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaac

SEQ ID NO: 94 - DNA, 699 bp, synthetic construct
  source 1..699: mol_type = other DNA
atggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagataa

SEQ ID NO: 95 - DNA, 32 bp, synthetic construct
  source 1..32: mol_type = other DNA
tagagggtatataatggaagctcgatttccag

SEQ ID NO: 96 - protein, 254 aa, synthetic construct
MLRLLLALNLFPSIQVTGGSSFFEDPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 97 - protein, 17 aa, synthetic construct
MLRLLLALNLFPSIQVT

SEQ ID NO: 98 - protein, 37 aa, synthetic construct
PTPAPTIASQPLSLRPEACRPAAGGAVHTRGLDFACD

SEQ ID NO: 99 - protein, 29 aa, synthetic construct
SKPFWVLVVVGGVLACYSLLVTVAFIIFW

SEQ ID NO: 100 - protein, 44 aa, synthetic construct
VRKRGRKKLLYIFKQPFMRPVQTTQEEDGCSCRFPEEEEGGCEL

SEQ ID NO: 101 - protein, 116 aa, synthetic construct
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAS

SEQ ID NO: 102 - protein, 19 aa, synthetic construct
ATNFSLLKQAGDVEENPGP

SEQ ID NO: 103 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 104 - protein, 201 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAAS

SEQ ID NO: 105 - protein, 4 aa, synthetic construct
RAKR

SEQ ID NO: 106 - protein, 4 aa, synthetic construct
SGSG

SEQ ID NO: 107 - protein, 20 aa, synthetic construct
QCTNYALLKLAGDVESNPGP

SEQ ID NO: 108 - protein, 376 aa, synthetic construct
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 109 - protein, 232 aa, synthetic construct
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

(19) United States
(12) Patent Application Publication
(10) Pub. No.: US 2026/0098255 A1
(43) Pub. Date: Apr. 9, 2026

(54) COMPOSITIONS AND METHODS FOR IDENTIFICATION OF VHH ANTIBODIES THAT BIND A TARGET ANTIGEN

(71) Applicants: The Broad Institute, Inc., Cambridge, MA (US); The General Hospital Corporation, Boston, MA (US); Dana-Farber Cancer Institute, Inc., Boston, MA (US)

(72) Inventors: Robert Manguso, Boston, MA (US); Gabriel Griffin, Boston, MA (US); Nelson Knudsen, Cambridge, MA (US); Hans Pope, Cambridge, MA (US)

(73) Assignees: The Broad Institute, Inc., Cambridge, MA (US); The General Hospital Corporation, Boston, MA (US); Dana-Farber Cancer Institute, Inc., Boston, MA (US)

(21) Appl. No.: 19/415,305

(22) Filed: Dec. 10, 2025

Related U.S. Application Data
(63) Continuation of application No. PCT/US2024/034097, filed on Jun. 14, 2024.
(60) Provisional application No. 63/508,232, filed on Jun. 14, 2023.

(51) Int. Cl. C12N 15/10; C07K 14/705; C07K 14/725; C12N 9/12

(52) U.S. Cl. CPC C12N 15/1037; C07K 14/7051; C07K 14/70521; C12N 9/1211; C12N 15/1082; C12N 15/1086; C07K 2319/70; C12Y 207/01021

(57) ABSTRACT

Compositions and methods that are useful for identification of VHH antibodies that bind a target antigen.

### CROSS-REFERENCE TO RELATED APPLICATION

This application is a continuation under 35 U.S.C. § 111(a) of PCT International Patent Application No. PCT/US2024/034097, filed Jun. 14, 2024, designating the United States and published in English, which claims priority to and the benefit of U.S. Provisional Application No. 63/508,232, filed Jun. 14, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Aug. 13, 2024, is named 167741-050001PCT_SL.xml and is 173,053 bytes in size.

### BACKGROUND

A VHH antibody, also known as a nanobody, is an antibody consisting of a single monomeric variable antibody domain. VHH antibodies are able to bind to a target antigen. VHH antibodies are smaller than other antibodies, such as multimeric antibodies composed of two or more polypeptide molecules. VHH antibodies typically have similar affinity to antigens as larger (e.g., multimeric) antibodies, but tend to be more heat-resistant and stable towards detergents and high concentrations of urea. Also, VHH antibodies do not show complement system triggered cytotoxicity because they lack an Fc region, and they are able to bind to hidden antigens that are not accessible to larger antibodies, for example to the active sites of enzymes.

Given the beneficial characteristics of VHH antibodies, there is a need for improved methods for the generation and screening of VHH antibodies to identify new VHH antibodies capable of binding an antigen.

### SUMMARY

As described below, the present disclosure features compositions and methods that are useful for identification of VHH antibodies that bind a target antigen.

In one aspect, the disclosure features a method for identifying a VHH antibody that binds a target antigen. The method involves (a) preparing a library of expression vectors encoding chimeric antigen receptors (CARs). Each CAR contains a VHH domain generated in response to an antigen of interest. The method further involves (b) expressing each member of the library of expression vectors in an immortalized immune cell. The immortalized immune cell contains a selection vector containing an activation induced promoter operably linked to a resistance gene and a sensitizing gene, where the resistance gene provides for positive selection with a positive selection agent and the sensitizing gene provides for negative selection with a negative selection agent. The method also involves (c) contacting the immune cell with an antigen that is not the antigen of interest and the negative selection agent. The method further involves (d) contacting the immune cell with the antigen of interest and the positive selection agent. The method results in the identification of chimeric antigen receptors containing VHH domains that selectively bind the target antigen.

In another aspect, the disclosure features a kit suitable for use in the method of any of the aspects of the disclosure, or embodiments thereof, where the kit contains the immortalized immune cell containing the selection vector.

In any aspect of the disclosure, or embodiments thereof, the immortalized immune cells are Jurkat cells.

In any aspect of the disclosure, or embodiments thereof, the antigen of interest is expressed on the surface of a cell. In any aspect of the disclosure, or embodiments thereof, the antigen of interest is associated with a neoplasia. In embodiments, the neoplasia is a cancer.

In any aspect of the disclosure, or embodiments thereof, the antigen of interest is a polypeptide.

In any aspect of the disclosure, or embodiments thereof, the VHH domains were generated in an animal exposed to an immunogenic composition containing an antigen presenting cell (APC) expressing the antigen of interest. In embodiments, the APC is a dendritic cell. In embodiments, the animal is a mouse. In embodiments, the animal belongs to the subfamily Camelinae. In embodiments, the animal belongs to a genus selected from one or more of Lama, Hemiauchenia, Palaeolama, Camelus, Camelops, or Paracamelus. In embodiments, the animal is a dromedary, a Syrian camel, a camel, a Bactrian camel, a wild Bactrian camel, a llama, a guanaco, an alpaca, or a vicuña.

In any aspect of the disclosure, or embodiments thereof, the method further involves sequencing one or more polynucleotides encoding the VHH domains that selectively bind the target antigen.

In any aspect of the disclosure, or embodiments thereof, the expression vectors each contain a promoter controlling expression of the encoded CAR. In embodiments, the promoter is a constitutive promoter. In embodiments, the promoter is an EFS promoter.

In any aspect of the disclosure, or embodiments thereof, the expression vectors are viral vectors. In embodiments, the viral vectors are lentiviral vectors.

In any aspect of the disclosure, or embodiments thereof, CAR contains from N-terminus to C-terminus, a CD28 signal peptide, a VHH domain, a CD28 transmembrane domain, a CD28 cytoplasmic domain, and a CD3ζ domain.

In any aspect of the disclosure, or embodiments thereof, the immortalized immune cell is an immune effector cell. In any aspect of the disclosure, or embodiments thereof, the immortalized immune cell is a T cell, NK cell, or precursors thereof. In any aspect of the disclosure, or embodiments thereof, the immune cell is a Jurkat cell.

In any aspect of the disclosure, or embodiments thereof, the activation induced promoter contains a nuclear factor of activated T cells response element (NFAT RE). In any aspect of the disclosure, or embodiments thereof, the activation induced promoter contains two or more tandem repeats of the NFAT RE. In any aspect of the disclosure, or embodiments thereof, the activation induced promoter contains three tandem repeats of the NFAT RE. In any aspect of the disclosure, or embodiments thereof, the activation induced promoter contains a minimal promoter.

In any aspect of the disclosure, or embodiments thereof, the selection vector contains a detectable reporter.

In any aspect of the disclosure, or embodiments thereof, the negative and positive selection genes encode a single polypeptide containing a self-cleaving peptide. In embodiments, the self-cleaving peptide is P2A or T2A.

In any aspect of the disclosure, or embodiments thereof, the resistance gene is a puromycin resistance gene and the positive selection agent contains puromycin.

In any aspect of the disclosure, or embodiments thereof, the sensitizing gene encodes an HSV thymidine kinase and the negative selection agent contains ganciclovir.

In any aspect of the disclosure, or embodiments thereof, the method further involves sequencing VHH domains encoded by the immune cells following exposure to the positive selection agent and/or the negative selection agent.

In any aspect of the disclosure, or embodiments thereof, the method further involves sequencing VHH domains encoded by the immune cells following exposure to both the positive selection agent and the negative selection agent.

The disclosure provides compositions and methods that are useful for identification of VHH antibodies that bind a target antigen. Compositions and articles defined by the disclosure were isolated or otherwise manufactured in connection with the examples provided below. Other features and advantages of the various aspects and embodiments of the disclosure will be apparent from the detailed description, and from the claims.

### Definitions

Unless defined otherwise, all technical and scientific terms used herein have the meaning commonly understood by a person skilled in the art to which the various aspects and embodiments of this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “alteration” is meant a change in the structure, sequence, expression levels or activity of a polynucleotide or polypeptide as detected by standard art known methods such as those described herein. The alteration can be an increase or a decrease. As used herein, an alteration includes a 10% change in expression levels, a 25% change, a 40% change, and a 50% or greater change in expression levels.”

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” or “antigen-binding domain” refers to an immunoglobulin molecule, a nanobody, or a fragment thereof that specifically binds to, or is immunologically reactive with, a particular antigen. Non-limiting examples of antibodies or antigen-binding domains include VHH antibodies, polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments, as well as engineered antibodies, which include CrossMabs (e.g., CrossMab^{Fabs}, CrossMab^{CH1-CL} and CrossMab^{VH-VL} formats), or fragments thereof. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

By “Chimeric Antigen Receptor” or alternatively a “CAR” is meant a polypeptide capable of providing an immune effector cell with specificity for a target cell. In embodiments, the target cell isa cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a VHH domain) during cellular processing and localization of the CAR to the cellular membrane. Embodiments of CARs are shown in FIGS. 9A and 9B.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include neoplasias, such as tumors and cancers. In various instances, the cancer is a lung cancer or an ovarian cancer.

By “effective amount” is meant the amount of an agent required to ameliorate the symptoms of a disease relative to an untreated patient. The effective amount of active compound(s) used to practice the various aspects and embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “ganciclovir” is meant a compound with the following structure:

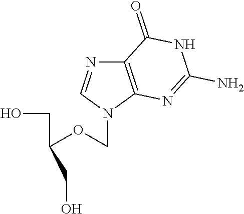

and corresponding to CAS No. 82410-32-0, or pharmaceutically acceptable salts or analogs thereof.

By “increase” is meant to alter positively by at least 5% relative to a reference. An increase may be by 5%, 10%, 25%, 30%, 50%, 75%, or even by 100%.

An “intracellular signaling domain,” refers to portion of a molecule that transduces a signal from the surface of the cell to the interior of the cell. The intracellular signaling domain generates a signal that promotes an immune effector function of a CAR containing cell, e.g., a CART cell. Examples of immune effector function, e.g., in a CART cell, include cytolytic activity and helper activity, including the secretion of cytokines.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid (e.g., a pLX311 plasmid or a pLX307 plasmid) or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a developmental state, condition, disease, or disorder. A non-limiting example of a marker is a mesothelin polypeptide.

By “mesothelin polypeptide” is meant a protein with at least about 85% amino acid sequence identity to Genbank Accession No. AAH03512.1, or a fragment thereof that is immunogenic in a subject. An exemplary mesothelin amino acid sequence from Homo Sapiens is provided below (GenBank: AAH03512.1):

>AAH03512.1 Mesothelin [Homo sapiens]
(SEQ ID NO: 1)
MALPTARPLLGSCGTPALGSLLFLLFSLGWVQPSRTLAGETGQAA
PLDGVLANPPNISSLSPRQLLGFPCAEVSGLSTERVRELAVALAQ
KNVKLSTEQLRCLAHRLSEPPEDLDALPLDLLLFLNPDAFSGPQA
CTRFFSRITKANVDLLPRGAPERQRLLPAALACWGVRGSLLSEAD
VRALGGLACDLPGRFVAESAEVLLPRLVSCPGPLDQDQQEAARAA
LQGGGPPYGPPSTWSVSTMDALRGLLPVLGQPIIRSIPQGIVAAW
RQRSSRDPSWRQPERTILRPRFRREVEKTACPSGKKAREIDESLI
FYKKWELEACVDAALLATQMDRVNAIPFTYEQLDVLKHKLDELYP
QGYPESVIQHLGYLFLKMSPEDIRKWNVTSLETLKALLEVNKGHE
MSPQVATLIDRFVKGRGQLDKDTLDTLTAFYPGYLCSLSPEELSS
VPPSSIWAVRPQDLDTCDPRQLDVLYPKARLAFQNMNGSEYFVKI
QSFLGGAPTEDLKALSQQNVSMDLATFMKLRTDAVLPLTVAEVQK
LLGPHVEGLKAEERHRPVRDWILRQRQDDLDTLGLGLQGGIPNGY
LVLDLSVQEALSGTPCLLGPGPVLTVLALLLASTLA.

By “mesothelin polynucleotide” is meant a nucleic acid molecule encoding a mesothelin polypeptide, as well as the introns, exons, 3′ untranslated regions, 5′ untranslated regions, and regulatory sequences associated with its expression, or fragments thereof. In embodiments, a mesothelin polynucleotide is the genomic sequence, cDNA, mRNA, or gene associated with and/or required for mesothelin expression. An exemplary mesothelin nucleotide sequence from Homo Sapiens is provided below (GenBank: BC003512.1:418-2283):

>BC003512.1:418-2283 Homo sapiens mesothelin,
mRNA (cDNA clone MGC: 10686 IMAGE: 3611296),
complete cds
(SEQ ID NO: 2)
ATGGCCTTGCCAACGGCTCGACCCCTGTTGGGGTCCTGTGGGACC
CCCGCCCTCGGCAGCCTCCTGTTCCTGCTCTTCAGCCTCGGATGG
GTGCAGCCCTCGAGGACCCTGGCTGGAGAGACAGGGCAGGCTGCA
CCCCTGGACGGAGTCCTGGCCAACCCACCTAACATTTCCAGCCTC
TCCCCTCGCCAACTCCTTGGCTTCCCGTGTGCGGAGGTGTCCGGC
CTGAGCACGGAGCGTGTCCGGGAGCTGGCTGTGGCCTTGGCACAG
AAGAATGTCAAGCTCTCAACAGAGCAGCTGCGCTGTCTGGCTCAC
CGGCTCTCTGAGCCCCCCGAGGACCTGGACGCCCTCCCATTGGAC
CTGCTGCTATTCCTCAACCCAGATGCGTTCTCGGGGCCCCAGGCC
TGCACCCGTTTCTTCTCCCGCATCACGAAGGCCAATGTGGACCTG
CTCCCGAGGGGGGCTCCCGAGCGACAGCGGCTGCTGCCTGCGGCT
CTGGCCTGCTGGGGTGTGCGGGGGTCTCTGCTGAGCGAGGCTGAT
GTGCGGGCTCTGGGAGGCCTGGCTTGCGACCTGCCTGGGCGCTTT
GTGGCCGAGTCGGCCGAAGTGCTGCTACCCCGGCTGGTGAGCTGC
CCGGGACCCCTGGACCAGGACCAGCAGGAGGCAGCCAGGGCGGCT
CTGCAGGGCGGGGGACCCCCCTACGGCCCCCCGTCGACATGGTCT
GTCTCCACGATGGACGCTCTGCGGGGCCTGCTGCCCGTGCTGGGC
CAGCCCATCATCCGCAGCATCCCGCAGGGCATCGTGGCCGCGTGG
CGGCAACGCTCCTCTCGGGACCCATCCTGGCGGCAGCCTGAACGG
ACCATCCTCCGGCCGCGGTTCCGGCGGGAAGTGGAGAAGACAGCC
TGTCCTTCAGGCAAGAAGGCCCGCGAGATAGACGAGAGCCTCATC
TTCTACAAGAAGTGGGAGCTGGAAGCCTGCGTGGATGCGGCCCTG
CTGGCCACCCAGATGGACCGCGTGAACGCCATCCCCTTCACCTAC
GAGCAGCTGGACGTCCTAAAGCATAAACTGGATGAGCTCTACCCA
CAAGGTTACCCCGAGTCTGTGATCCAGCACCTGGGCTACCTCTTC
CTCAAGATGAGCCCTGAGGACATTCGCAAGTGGAATGTGACGTCC
CTGGAGACCCTGAAGGCTTTGCTTGAAGTCAACAAAGGGCACGAA
ATGAGTCCTCAGGTGGCCACCCTGATCGACCGCTTTGTGAAGGGA
AGGGGCCAGCTAGACAAAGACACCCTAGACACCCTGACCGCCTTC
TACCCTGGGTACCTGTGCTCCCTCAGCCCCGAGGAGCTGAGCTCC
GTGCCCCCCAGCAGCATCTGGGCGGTCAGGCCCCAGGACCTGGAC
ACGTGTGACCCAAGGCAGCTGGACGTCCTCTATCCCAAGGCCCGC
CTTGCTTTCCAGAACATGAACGGGTCCGAATACTTCGTGAAGATC
CAGTCCTTCCTGGGTGGGGCCCCCACGGAGGATTTGAAGGCGCTC
AGTCAGCAGAATGTGAGCATGGACTTGGCCACGTTCATGAAGCTG
CGGACGGATGCGGTGCTGCCGTTGACTGTGGCTGAGGTGCAGAAA
CTTCTGGGACCCCACGTGGAGGGCCTGAAGGCGGAGGAGCGGCAC
CGCCCGGTGCGGGACTGGATCCTACGGCAGCGGCAGGACGACCTG
GACACGCTGGGGCTGGGGCTACAGGGCGGCATCCCCAACGGCTAC
CTGGTCCTAGACCTCAGCGTGCAAGAGGCCCTCTCGGGGACGCCC
TGCCTCCTAGGACCTGGACCTGTTCTCACCGTCCTGGCACTGCTC
CTAGCCTCCACCCTGGCCTGA.

By “neoplasia” is meant a disease or disorder characterized by excess proliferation or reduced apoptosis. In embodiments, a neoplasia is a cancer or tumor. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In embodiments, the neoplasia may be a liquid tumor such as, for example, leukemia or lymphoma. In embodiments, the cancer is a colon, kidney, lung, pancreatic, renal (e.g., renal cell carcinoma or clear renal cell carcinoma), or skin cancer (e.g., a melanoma).

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. Various aspects and embodiments of the disclosure embrace sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “puromycin” is meant a compound with the following structure:

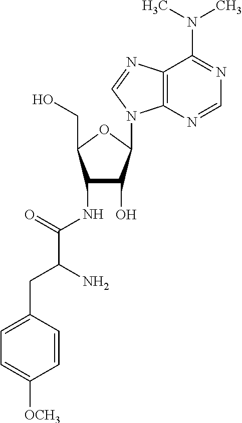

and corresponding to CAS No. 53-79-2, or pharmaceutically acceptable salts or analogs thereof.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference is a cell that does not comprise a polymer of the disclosure, such as a chimeric antigen receptor comprising a VHH domain, a puromycin resistance polypeptide, and/or an HSV thymidine kinase. In some cases, a reference is a cell containing a chimeric antigen receptor comprising a VHH domain that does not bind a target antigen. In some cases, a reference is an antigen presenting cell that does not express a target antigen (e.g., a DC2.4 cell), or a cell co-cultured with the antigen presenting cell. In various instances, a reference is a cell or cell population cultured in the absence of a selective condition, such as the presence of a toxic agent that can be neutralized enzymatically (e.g., by a puromycin resistance polypeptide) or a non-toxic agent that can be converted into a toxic agent enzymatically (e.g., by a HSV thymidine kinase), such as puromycin or ganciclovir, respectively.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 10 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In embodiments, a reference sequence is a VHH antibody or a fragment thereof that does not bind a target antigen. In some cases, a reference sequence is a VHH antibody or a fragment thereof that binds a target antigen.

By “specifically binds” is meant in the context of an antibody or other polypeptide capture molecule recognizes and binds a polypeptide of the disclosure, but which does not substantially recognize and bind other molecules in a sample. In embodiments, the capture molecule is a VHH domain or a fragment thereof. A VHH domain or fragment thereof that specifically binds to an antigen will bind to the antigen with a K_{D} of less than 100 nM. For example, a VHH domain or fragment thereof that specifically binds to an antigen will bind to the antigen with a K_{D} of up to 100 nM (e.g., between 1 pM and 100 nM). A VHH domain or fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select a VHH domain or fragment thereof that specifically immunoreactive with a particular protein or carbohydrate. For example, solid-phase ELISA immunoassays are routinely used to select VHH domains or fragments thereof specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodiun citrate, about less than about 500 mM NaCl and 50 mM trisodium citrate, or about less than about 250 mM NaCl and 25 mM trisodiun citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Transduction” refers to a process by which a polynucleotide is introduced or transferred into a cell. In an embodiment, the DNA or polynucleotide transduced into a cell is stably expressed in the cell. In some cases, the virus or virus vector is said to infect a cell.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to a polynucleotide suitable for delivery of a gene sequence to a cell, or to a virus particle. Non-limiting examples of vectors include plasmids (e.g., a pLX311 plasmid or a pLX307 plasmid) and cosmids. A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a heterologous gene, such as a therapeutic gene, or a reporter gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region, and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site to direct efficient transcription of a gene carried on the expression vector. Vectors, such as the viral particles described herein, may also be referred to as expression vectors.

As used herein, the term “vehicle” refers to a solvent, diluent, or carrier component of a pharmaceutical composition.

By “viral particle” is meant an agent capable of infecting a cell and that exists as an independent particle containing a core viral genome or polynucleotide, a capsid, which surrounds the genetic material and protects it, and an envelope of lipids surrounding the capsid. A viral particle may refer to the form of a virus before it infects a cell and becomes intracellular, or to the form of the virus that infects a cell.

By “VHH domain” is meant an antigen binding domain of a heavy chain only antibody or an antigen binding fragment thereof.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic overview of a screen for the identification of VHH antibodies (nanobodies) specific for an antigen. Antigen presenting cells (APCs) expressing an antigen of interest are used to vaccinate mice capable of producing nanobodies. VHH libraries produced by the vaccinated mice are screened to identify VHH antibodies that bind the antigen of interest.

FIGS. 2A and 2B provide a flow cytometry scatter plot and a bar graph demonstrating that mice administered antigen-presenting cells expressing ovalbumin (OVA) generated effective T-cell and B-cell/antibody responses against OVA. The mice were vaccinated with dendritic cells (DC2.4) engineered to constitutively express OVA. FIG. 2A provides a flow cytometry scatter plot showing OVA specific (tet+) CD8 T-cell responses in peripheral blood mononuclear cells (PBMCs) (uncircled in boxed region) from vaccinated mice. The PBMCs were admixed with positive-control OT-I cells (circled). FIG. 2B provides a bar graph showing results from an enzyme-linked immunosorbent assay (ELSA) showing that anti-OVA IgG antibodies were present in serum from vaccinated mice. In FIG. 2B “PBS” indicates serum from mice administered phosphate buffered saline, “DC2.4 (no OVA)” indicates serum from mice administered DC2.4 cells that did not express OVA, and “DC2.4 (+OVA)” indicates serum from mice administered DC2.4 cells expressing+OVA. A549 lung carcinoma cells were used as a positive control.

FIG. 3 provides a schematic overview of a strategy for screening single domain antibodies (VHHs) against cell surface antigens using nanomice and chimeric antigen receptors (CARs). Nanomice were vaccinated using dendritic cells (DCs) expressing a target antigen. VHH-encoding polynucleotide sequences were then prepared from nanobody-producing B cells in the mice. The VHH-encoding polynucleotide sequences were used to prepare libraries of lentiviral vectors containing polynucleotide sequences encoding chimeric antigen receptors containing the VHH antibodies as their antigen-binding domains.

FIG. 4 provides a schematic diagram relating to whole-cell vaccination using a DC2.4 cell line. Antigens of interest can be introduced to the DC2.4 cells through lentiviral transduction using a lentiviral vector containing a polynucleotide encoding an antigen of interest. Antigens expressed in the DC2.4 cells are presented by MHC class I and/or class II molecules on the surface of the cells. FIG. 4 provides a schematic diagram showing a strategy for immunizing animals using DC2.4 cells expressing a target antigen. The DC2.4 cells are stimulated with CpG oligodeoxynucleotides for about 4 hours prior to vaccinating an animal (e.g., a mouse) with about 5 million cells by intraperitoneal injection (IP) and subcutaneous injection. After about two weeks, the animals are administered a boost vaccination. After about two additional weeks, cells (e.g., antibody-producing B cells) are harvested from the animals, or the animals are administered further boost vaccinations.

FIG. 5 provides flow cytometry scatter plots showing that whole cell vaccination of mice with DC2.4 cells expressing ovalbumin (OVA) induced antigen-specific T cell expansion. In FIG. 5, “DC2.4 Vax” indicates cells from mice vaccinated using DC2.4 cells that did not express OVA, “DC2.4-OVA Vax” indicates cells from mice vaccinated using DC2.4 cells expressing OVA, and “OT-1” indicates OT-1 cells, which were used as a positive control. The boxed are in the flow cytometry scatter plots of FIG. 5 surrounds a region corresponding to Tet+, CD8+ cells.

FIG. 6 provides a plot showing results from enzyme-linked immunosorbent assays demonstrating that whole cell vaccination of mice using DC2.4 cells expressing ovalbumin (OVA) induced OVA-specific (i.e., antigen-specific) antibody responses. In FIG. 6 “OD” indicates “optical density,” PBS indicates cells from mice vaccinated using phosphate buffered saline, “DC2.4 Vax” indicates cells from mice vaccinated using DC2.4 cells that did not express OVA, and “DC2.4-OVA Vax” indicates cells from mice vaccinated using DC2.4 cells expressing OVA.

FIGS. 7A and 7B provide flow cytometry histograms showing that nanomice vaccinated using DC2.4 cells expressing mesothelin produced anti-mesothelin nanobodies.

FIG. 7A provides a flow cytometry histogram confirming that DC2.4 cells transduced with mesothelin expression constructs expressed the mesothelin and presented mesothelin antigens on their surface. In FIG. 7A, “DC2.4 PLX311 Meso” indicates DC2.4 cells transduced with a pLX311 plasmid encoding mesothelin, “DC2.4 PLX307 Meso” and “DC2.4 PLX307 Meso” indicate DC2.4 cells transduced with a pLX307 plasmid encoding mesothelin, “A549 (+ctrl)” indicates A549 lung carcinoma cells, “DC2.4 NT” indicates untransduced DC2.4 cells, and “Unstained” indicates cells that were not immunostained. FIG. 7B provides a flow cytometry histogram demonstrating that nanomice vaccinated using DC2.4 cells expressing mesothelin contained anti-mesothelin nanobodies in their blood serum. In FIG. 7B, “Unstained” indicates unstained cells, “Secondary Only” indicates cells immunostained only using the secondary antibody, “DC2.4” indicates serum from mice vaccinated using DC2.4 cells that did not express mesothelin, and “DC2.4 Mesothelin” indicates serum from mice vaccinated using DC2.4 cells expressing mesothelin.

FIG. 8 provides a schematic diagram presenting a strategy for screening a library of polynucleotide sequences encoding VHH antibodies for those antibodies that bind a target antigen. The screen is a chimeric-antigen receptor—(CAR) based screen. The screen involves expressing nanobody-containing CARs on the surface of Jurkat cells and then subjecting the Jurkat cells to a negative antigen selection using ganciclovir and a positive antigen selection using puromycin followed by sequencing of VHH-encoding polynucleotide sequences associated with Jurkat cells that passed both stages of the screen.

FIGS. 9A and 9B provide schematic diagrams showing an embodiment of a chimeric antigen receptor and polynucleotide encoding the same suitable for use in the methods of the disclosure. In embodiments, the polynucleotide shown in FIG. 9B is part of a plasmid called “pSLCAR-VHH.” In FIG. 9B, “EFS” indicates an EF-1 alpha short (EFS) promoter, “CD28sp” indicates a signal peptide from cluster of differentiation 28 (CD28), “CD8h” indicates a hinge domain derived from cluster of differentiation 8 (CD8), “CD28tm” indicates a transmembrane domain derived from cluster of differentiation 28, “CD28cyto” indicates a cytoplasmic domain derived from cluster of differentiation 28 (CD28), “CD3ζ” indicates an activation signaling domain derived from cluster of differentiation CD3, “P2A” indicates a self-cleaving peptide, “eGFP” indicates the fluorescent protein “enhanced green fluorescent protein,” and “BpiI” represents the restriction enzyme BpiI from Bacillus pumillus SW 4-3 with isoschizomers (i.e., restriction endonucleases that recognize the same DNA sequence and make the same cut) BbsI, BbsI-HF®, BpuAI, and BstV2I and recognizing the following sequence, where N₂ and N₆ indicate any 2 or 6 nucleotides, respectively, and the upward and downward arrows indicate the location the site nicked by the restriction enzyme on the indicated DNA stand:

5′ G A A G A C N₂ ↓ 3'
3' C T T C T G N₆ ↑ 5'

FIGS. 10A and 10B provide flow cytometry scatter plots showing that chimeric antigen receptors (CARs) containing VHH domains that bind mesothelin activated signaling (i.e., cluster of differentiation 69 (CD69) activation) in Jurkat cells expressing the same when the Jurkat cells were co-cultured with cells surface-expressing mesothelin (i.e., mesothelin+ cells). In FIGS. 10A and 10B, “Mesothelin⁺ DC2.4” indicates DC2.4 cells that express mesothelin, “Untransduced Jurkat” cells indicates Jurkat cells that do not express a chimeric antigen receptor, “Meso-Nb CAR Jurkat” indicates Jurkat cells expressing a chimeric antigen receptor (CAR) containing an anti-mesothelin nanobody (i.e., VHH) domain, and “Control DC2.4” indicates DC2.4 cells that do not express mesothelin. In FIGS. 10A and 10B, activated cells fall within quadrant 2 (Q2), and the numbers below each quadrant label (i.e., Q1, Q2, Q3, or Q4) indicate the percent of total cells counted that fell within the indicated quadrant. In FIGS. 10A and 10B, the term “Meso-Nb CAR” indicates a chimeric antigen receptor (CAR) containing an anti-mesothelin nanobody (Nb) as an antigen-binding domain.

FIG. 11 provides a schematic diagram showing a screening construct used in the positive and negative selections of the methods of the disclosure. The screening construct contained three nuclear factor of activated T cells (NFAT) response element (RE) sequences in tandem upstream of a minimal promoter controlling transcription from a polynucleotide sequence encoding a polypeptide. The polypeptide contained a puromycin resistance (PuroR) polypeptide capable of inactivating puromycin, an HSV Thymidine Kinase (HSV-TK) polypeptide capable of converting ganciclovir into a toxic product, and the fluorescent protein mKate, each linked to one another by self-cleaving peptides (i.e., P2A and T2A). The NFAT response element showed only low levels of basal activation, if any, in Jurkat cells. The puromycin resistance polypeptide (PuroR) allowed for positive selection for cells expressing chimeric antigen receptors containing a VHH domain binding a target antigen by contacting the cells with the target antigen and puromycin. The HSV thymidine kinase allowed for negative section against cells expressing chimeric antigen receptors containing a VHH domain that were activated when contacted with a non-target antigen or no antigen by selecting the cells with ganciclovir in the absence of a target antigen. The red fluorescent protein, mKate, was used to monitor construct expression.

FIG. 12 provides flow cytometry scatter plots showing results from a negative selection before and after selecting cells with ganciclovir. Jurkat cells collectively expressing a library of chimeric antigen receptors containing different VHH domains produced by mice vaccinated using DC2.4 cells expressing mesothelin were transduced with the expression construct of FIG. 11. The Jurkat cells were then co-cultured with DC2.4 cells that did not express mesothelin. Flow cytometry was used to measure levels of construct expression, as determined by measuring mKate fluorescence, before and after selecting the co-cultured cells with ganciclovir (i.e., after the negative selection). CAR-expressing Jurkat cells activated in the presence of the DC2.4 cells were killed when the cells were exposed to ganciclovir (compare the left and right plots of FIG. 12). The boxed region in FIG. 12 indicates the region of the plots corresponding to mKate expressing cells, and the numbers below the label “mKate+” indicates the percent of total counted cells that were measured as expressing mKate.

FIG. 13 provides flow cytometry scatter plots showing results form a positive selection screen before and after selecting cells with puromycin. Jurkat cells that passed the negative selection described in FIG. 12 were then subjected to a positive selection to identify those Jurkat cells expressing chimeric antigen receptors containing VHH domains capable of binding a mesothelin antigen. The left plot of FIG. 13 shows mKate expression in cells grown in the absence of DC2.4 cells expressing mesothelin, the middle plot shows expression of mKate in cells co-cultured with DC2.4 cells expressing mesothelin before being selected with puromycin, and the right plot shows expression of mKate in cells co-cultured with DC2.4 cells expressing mesothelin after being selected with puromycin (i.e., after positive selection). The boxed region in FIG. 13 indicates the region of the plots corresponding to mKate expressing activated (i.e., CD69+) Jurkat cells, and the numbers below the label “CD69+mKate+” indicates the percent of total counted activated cells (CD69+) that were measured as expressing mKate. The selection enriched for those cells activated by the DC2.4 cells expressing mesothelin.

FIG. 14 provides a schematic diagram, which is partially described in FIG. 93, showing that ˜350 bp polynucleotide molecules encoding VHH domain of the chimeric antigen receptors in the Jurkat cells were amplified using PCR and subsequently sequenced using next generation sequencing (NGS) by MiSeq.

FIGS. 15A-15H present a series of flow cytometry scatter plots demonstrating that the negative and positive selection screens presented herein were effective in identifying VHH antibodies capable of selectively binding an antigen of interest (e.g., mesothelin). FIGS. 15A-15D provide flow cytometry scatter plots showing activation by A375 cells (FIGS. 15A and 15B) or A20 cells (FIGS. 15C and 15D) of Jurkat CAR T cells subsequent to being subjected to a negative selection involving co-culture in ganciclovir-containing medium with either A20 cells (FIGS. 15A and 15D) or A375 cells (FIGS. 15B and 15C). FIGS. 15E-15H provide flow cytometry scatter plots showing enrichment of Jurkat CAR T cells capable of being activated by DC2.4 cells surface expressing mesothelin during a positive selection involving co-culturing the DC2.4 cells in medium containing puromycin (FIG. 15G). As controls, the cells were either not cocultured with any cells (FIG. 15E), the cells were co-cultured in the absence of puromycin or ganciclovir (FIG. 15F), or the cells were co-cultured in the presence of ganciclovir but in the absence of puromycin (FIG. 15H). In FIGS. 15A-15H, the term “A375-Meso” indicates mesothelin-expressing A375 cells and the term “A20” indicates A20 cells, which surface-expressed mouse major histocompatability complex II (mMHCII) but did not surface-express mesothelin. In each of FIGS. 15A-15H, the y-axis measures GFP fluorescence, which was directly correlated with chimeric antigen receptor expression in the cells, and the x-axis measures mKate expression, which was directly correlated with activation of CAR T cells by an antigen. The Jurkat CAR T cells subjected to the positive and negative screens expressed a library of chimeric antigen receptors containing as their antigen binding domains different VHH domains (see Table 1), and the screening construct shown in FIG. 11.

FIG. 16 presents a plot confirming the results of FIGS. 15B and 15G through next-generation sequencing of VHH domain sequences. In FIG. 16, “Input” indicates baseline change in VHH chimeric antigen receptor abundance in the Jurkat CAR T cells grown without being subjected to any co-culture or selection. The VHH domain CD15 is not represented in FIG. 16.

FIG. 17 provides a plasmid map for a plasmid encoding a chimeric antigen receptor of the disclosure.

FIG. 18 provides a plasmid map for a plasmid encoding a screening construct of the disclosure.

### DETAILED DESCRIPTION

The disclosure features compositions and methods that are useful for identification of VHH antibodies that bind a target antigen.

The various aspects and embodiments of the disclosure are base at least in part upon, among other things, the discovery of a method, as described further in the Examples provided herein, for screening libraries of VHH antibodies for those antibodies that are capable of binding a target antigen. In various instances, the screening methods involve immunization of a nanobody producing animal (e.g., a nanomouse) with a target antigen. In some cases, the animals are vaccinated using antigen presenting cells (APCs) (e.g., dendritic cells (DCs)) expressing the target antigen. The nanobodies produced by the vaccinated animals are screened in a chimeric antigen receptor—(CAR) based screen involving a positive selection step and a negative selection step to identify VHH antibodies capable of binding a target antigen.

### VHH Antibodies

In various aspects, the disclosure provides methods for the identification of VHH antibodies, also known as “nanobodies,” capable of binding a target antigen, as well as polypeptides containing VHH domains or polynucleotides encoding the same. In embodiments, the VHH antibodies bind an antigen associated with a disease or disorder (e.g., mesothelin), such as a neoplasia. In embodiments, the VHH binds an antigen associated with a target cell. In embodiments, the target cell is a neoplastic cell.

VHH domains are derived from nanobodies. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, a cloned and isolated VHH domain is a stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, nanobodies show high target specificity, high affinity for their target, and low inherent toxicity. However, like small molecule drugs they can inhibit enzymes and readily access receptor clefts. Furthermore, Nanobodies are stable, can be administered by means other than injection (see, e.g., International Patent Application No. WO 2004/041867 A2, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula, or Pichia) (see, e.g., U.S. Pat. No. 6,838,254, which is herein incorporated by reference in its entirety).

### Methods for Identifying VHH Antibodies

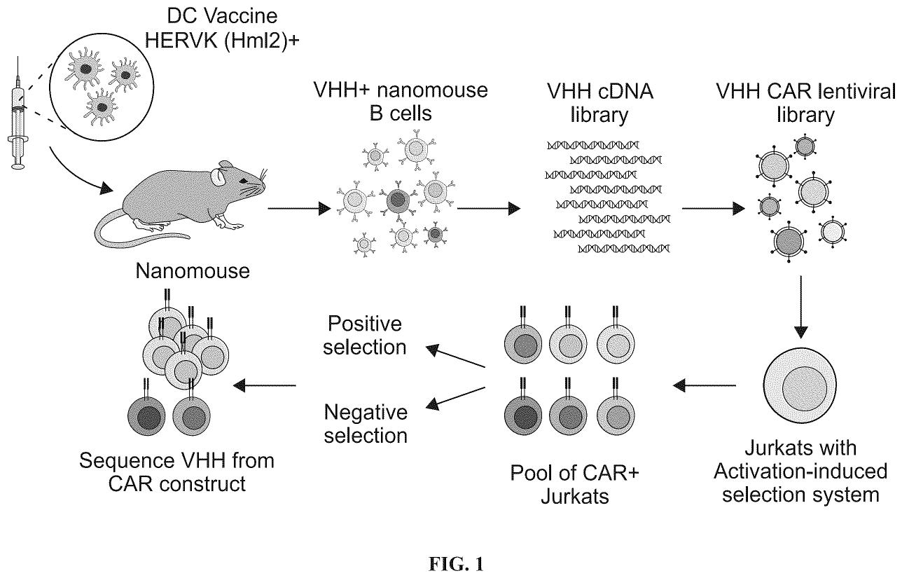

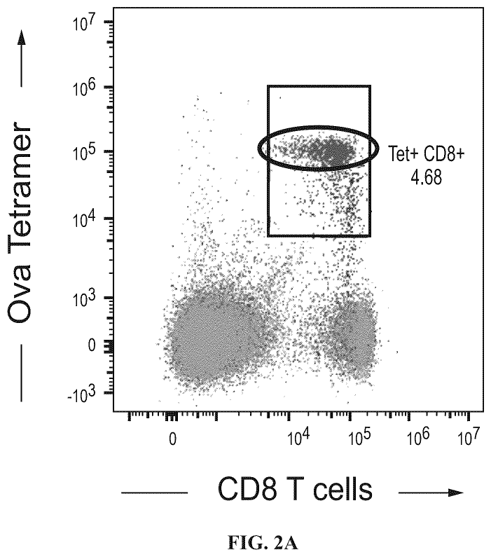

In various aspects, the disclosure provides methods for discovery of nanobodies specific for a target antigen. In embodiments, the methods involve the use of a chimeric antigen receptor—(CAR) based screen. Embodiments of the methods of the disclosure are shown in FIGS. 1, 3, and 8. The methods involve vaccinating an animal capable of producing VHH antibodies with a target antigen, preparing chimeric antigen receptor (CAR) cells containing chimeric antigen receptors containing VHH antibodies produced in response to the target antigen in the animal, and passing the CAR cells through a positive selection and a negative selection to identify those chimeric antigen receptors capable of binding the target antigen (FIG. 1). In some instances, the cell-based screening methods provided herein allow for selection of functional antibody sequences. In some cases, the methods provided herein facilitate identification of functional antibodies for use in pre-clinical or clinical studies.

In various embodiments, the methods of the disclosure involve administering a target antigen to an animal capable of producing VHH antibodies capable of binding the antigen as part of an immune response to the antigen. In various instances, the target antigen is a marker associated with a disease or disorder (e.g., a neoplasia). Non-limiting examples of animals suitable for use in the methods of the disclosure include those described in Xu, et al. “Nanobodies from camelid mice and llamas neutralize SARS-CoV-2 variants,” Nature, 595:278-282 (2021), the disclosure of which is incorporated herein in its entirety for all purposes. In embodiments, the animal is a nanomouse (i.e., a camelid mouse) or a camelid (e.g., a dromedary, a Syrian camel, a camel, a Bactrian camel, a wild Bactrian camel, a llama, a guanaco, an alpaca, or a vicuña). In embodiments, the animal is a member of the subfamily Camelinae. In some instances, the animal belongs to the genus Lama, Hemiauchenia, Palaeolama, Camelus, Camelops, or Paracamelus.

In various cases, the antigen is introduced to the animal as a purified polypeptide or as a live vaccine. In some cases, the live vaccine contains an antigen presenting cell that expresses the target antigen. Non-limiting examples of cells suitable for use as live vaccines include dendritic cells (e.g., a DC2.4 cell). Further non-limiting examples of antigen presenting cells include B cells. In some cases, the antigen presenting cells expressing the target antigen are a transposase or lentiviral cell line with a polynucleotide encoding the target antigen integrated into the genome thereof. Methods for integrating polynucleotide sequences into the genome of a cell are known to the skilled practitioner (see, e.g., Sandoval-Villegas, et al. “Contemporary Transposon Tools: A Review and Guide through Mechanisms and Applications of Sleeping Beauty, piggyBac and Tol2 for Genome Engineering,” International Journal of Molecular Sciences, 22:5084 (2021) doi.org/10.3390/ijms22105084; Cong, L., Zhang, F. (2015). Genome Engineering Using CRISPR-Cas9 System. In: Pruett-Miller, S. (eds) Chromosomal Mutagenesis. Methods in Molecular Biology, vol 1239. Humana Press, New York, NY. doi: 10.1007/978-1-4939-1862-1_10; and Froelich, et al. “Lentiviral vectors for Immune Cells Targeting,” Immunopharmacol Immunotoxicol, 32:208-218 (2010), the disclosures of which are incorporated herein by reference in their entireties for all purposes).

It can be advantageous in some instances to use a live vaccine to administer a target antigen to a cell if it is difficult to purify the target antigen or express the target antigen in a host cell. In embodiments, expressing the target antigen in the antigen presenting cells (e.g., dendritic cells) eliminates the need to generate a soluble and/or correctly folded antigen with which to vaccinate the animals. In embodiments, the target antigen is a cell surface protein, or a fragment thereof.

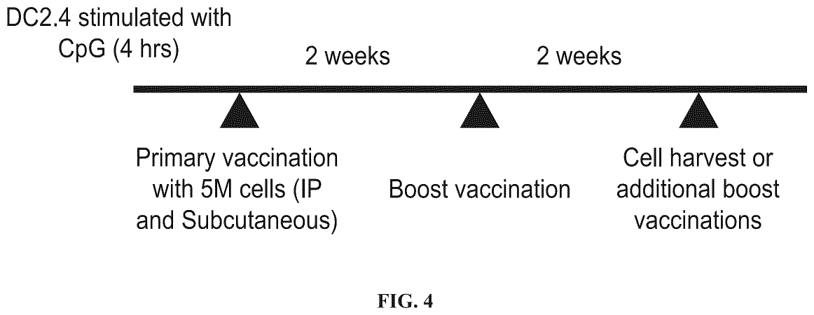

The methods of the disclosure involve immunizing the animal capable of producing VHH antibodies with the live vaccine or purified antigen. A non-limiting example of a vaccination schedule is provided in FIG. 4, where the animal is administered to vaccinations each separated by two weeks followed by a sample-collection two weeks following the second vaccination. In some instances, the animals are administered 1, 2, 3, 4, 5, 6, 7, 8, 9, or 10 vaccinations with the live vaccine or purified antigen, where each subsequent vaccination following a first vaccination may individually take place 1, 2, 3, 4, 5, 6, 7, 8, 9, or 10 weeks following the previous vaccination. In some cases, each subsequent vaccination following a first vaccination may individually take place no more than 1, 2, 3, 4, 5, 6, 7, 8, 9, or 10 weeks following the previous vaccination. In some cases, a sample comprising immune cells and/or VHH antibodies is collected from the animals about or at least about 1, 2, 3, 4, 5, 6, 7, 8, 9, or 10 weeks following the last vaccination. In some cases, the sample is collected from the animals not more than about 1, 2, 3, 4, 5, 6, 7, 8, 9, or 10 weeks following the last vaccination. In some cases, the sample collected from the animal is a blood sample containing mRNA encoding VHH antibodies produced as part of an immune response to the antigen.

In some cases, a live vaccine is stimulated prior to vaccination of an animal. It can be advantageous, for example to stimulate dendritic cells expressing a target antigen with a stimulating agent for about or at least about 1, 2, 3, 4, 5, 6, 7, 8, 9, 10, 11, or 12 hours prior to vaccination. It can be advantageous, for example to stimulate dendritic cells expressing a target antigen with a stimulating agent for no more than about 1, 2, 3, 4, 5, 6, 7, 8, 9, 10, 11, or 12 hours prior to vaccination. In some cases, the stimulating agent is a CpG oligodeoxynucleotide.

The methods of the disclosure involve in various embodiments preparing cDNA from mRNA expressed in the vaccinated animal. In embodiments, the methods involve amplifying from the cDNA using a polymerase chain reaction polynucleotide molecules encoding a VHH domain or a functional fragment thereof. The primers used in the polymerase chain reaction are so selected as to produce amplicons that each contain a restriction enzyme recognition at their 5′ and a restriction enzyme recognition sequence at their 3′ end. In some cases, the restriction enzyme recognition sequences are recognized by the same restriction enzyme. Non-limiting examples of restriction enzymes suitable for use in the methods of the disclosure include BpiI, BbsI, BbsI-HF®, BpuAI, and BstV2I.

The methods of the disclosure involve creating a library of chimeric antigen receptors by restriction digesting the amplicons encoding the VHH domains and cloning the VHH domain encoding polynucleotides in-frame into an expression construct encoding a chimeric antigen receptor so that the VHH becomes the antigen-binding domain of the encoded chimeric antigen receptor. In embodiments, the methods of the disclosure allow for a pooled cloning/screening approach without any need for pairing of immunoglobulin (e.g., IgG) heavy and light chains because VHH domains can be directly cloned into a chimeric antigen receptor. In some cases, the expression construct contains a promoter described herein, such as an EFS promoter that controls expression of the chimeric antigen receptor.

The methods involve introducing the expression construct into an immune cell. The expression construct can be introduced into the immune cell using any technique available to a skilled practitioner (e.g., electroporation or use of a vector) described herein. In some instances, the cells are transduced with the expression construct using a lentiviral vector. Non-limiting examples of immune cells include Jurkat cells or any other immune effector cell capable of expressing a functional chimeric antigen receptor on the surface thereof that is capable of generating an response when bound by an antigen.

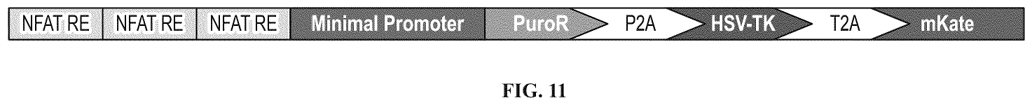

The immune cells encode a screening construct, such as that shown in FIG. 11. The screening construct contains a promoter that is activated in response to the chimeric antigen receptor binding an antigen. In an embodiment, the expression construct contains a single nuclear factor of activated T cells (NFAT) response element (RE) or 2, 3, 4, or 5 tandem repeats thereof upstream of a minimal promoter controlling transcription from a polynucleotide sequence encoding a polypeptide, such that expression of the polypeptide is increased from low or undetectable basal levels when the chimeric antigen receptor binds an antigen. The polypeptide encodes two functional polypeptides fused to one another by a self-cleaving peptide (e.g., a P2A or T2A self-cleaving peptide). One of the functional polypeptides (e.g., a puromycin resistance (PuroR) polypeptide) is capable of neutralizing an agent that is toxic to the immune cell. The other of the functional polypeptides (e.g., an HSV thymidine kinase (HSV-TK)) is capable of converting a non-toxic agent (e.g., ganciclovir) into an agent that is toxic to the immune cell.

The immune cells containing the library of chimeric antigen receptors containing the different VHH domains produced by the vaccinated animal are subjected to a positive screen and then to a negative screen, where the order of the screens is not necessarily limiting.

The positive screen involves contacting the CAR-expressing cells with the target antigen and the agent that is inactivated by one of the functional polypeptides expressed from the screening construct. Cells expressing chimeric antigen receptors containing VHH domains that do not bind the target antigen are killed in the positive screen. Therefore, the positive screen enriches for cells expressing chimeric antigen receptors containing VHH domains capable of binding the target antigen. In some cases, the target antigen is presented to the cells by contacting the cells with the same cells or purified polypeptide used to immunize the animal.

The negative screen involves growing the CAR-expressing cells in the absence of the target antigen and/or co-culturing the CAR-expressing cells with cells from the same cell line of antigen presenting cells used in the live vaccine, but that do not express the target antigen, and selecting the CAR-expressing cells with the agent that is converted to a toxic agent by one of the functional polypeptides expressed from the screening construct. Cells expressing chimeric antigen receptors containing VHH domains that bind to antigens other than the target antigen (e.g., an antigen displayed on the surface of the antigen presenting cells that do not express the target antigen) and that bind an antigen or are otherwise activated in the negative screen are killed in the negative screen. Therefore, the negative screen enriches for cells expressing chimeric antigen receptors containing VHH domains that do not bind an antigen present in the negative screen.

Following the positive and negative screen, the methods of the disclosure involve sequencing polynucleotides encoding the VHH domain of the chimeric antigen receptors encoded by the CAR-expressing cells. Any sequencing method available to a skilled practitioner or described herein may be used to sequence the polynucleotides.

In embodiments, the methods of the disclosure allow for identification of VHH antibodies capable of binding a target antigen within 1, 2, 3, 4, 5, 6, 7, 8, 9, or 10 months of first vaccination of an animal.

In embodiments, the VHH antibodies identified using the methods provided herein can be linked to agents (e.g., polypeptides) to be delivered to a site containing an antigen to which the VHH antibodies bind. For example, the VHH antibodies can be used to label a target antigen present in a sample by linking the VHH antibody to a detectable moiety, such as a dye or a fluorescent polypeptide. In embodiments, the VHH antibodies are linked to a drug to create an antibody-drug conjugate (ADC) (see, e.g., Birrer, et al. “Antibody-Drug Conjugate-Based Therapeutics: State of the Science,” JNCI, 111:538-549 (2019), the disclosure of which is incorporated herein in its entirety for all purposes) capable of selectively delivering the conjugated drug to the site of a target antigen. For example, the ADC can be used to deliver an agent to a target tissue or cell surface-expressing a target antigen.

### Delivery of Polynucleotides to a Cell

A cell of the disclosure, its progenitor or its in vitro-derived progeny, can contain a heterologous nucleotide sequence encoding genes to be expressed. Insertion of one or more pre-selected nucleotide molecules can be accomplished by homologous recombination or by viral integration into the host cell genome. The desired nucleotide molecule can also be incorporated into the cell, particularly into its nucleus, using a plasmid expression vector and a nuclear localization sequence. Methods for directing nucleotide molecules to the nucleus have been described in the art. The nucleotide molecules can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into a cell and is a standard method of DNA transfer to those of skill in the art. DEAE-dextran transfection, which is also known to those of skill in the art, may be advantageously used rather than calcium phosphate transfection where transient transfection is desired, as it is often more efficient. Since the cells of the present disclosure can be isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells of the present disclosure can also be genetically modified using electroporation.

Liposomal delivery of nucleotide molecules to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[1-(2, 3-dioleyloxy)propyl]-N—N—N-trimethyl ammonia chloride and DOPE. Liposomes can carry nucleotide molecules, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of nucleotide molecules into primary and established mammalian cell lines using lipopolyamine-coated nucleotide molecules can be used to introduce target DNA into the lymphatic endothelial progenitor cells described herein.

Naked plasmid DNA can be injected directly into a tissue comprising cells of the disclosure. This technique has been shown to be effective in transferring plasmid DNA to skeletal muscle tissue, where expression in mouse skeletal muscle has been observed for more than 19 months following a single intramuscular injection. More rapidly dividing cells take up naked plasmid DNA more efficiently. Therefore, it is advantageous to stimulate cell division prior to treatment with plasmid DNA. Microprojectile gene transfer can also be used to transfer nucleotide molecules into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Transducing viral vectors (e.g., retroviral vectors (e.g., lentiviral vectors), alphaviral vectors (e.g., Sindbis vectors), adenoviral vectors, herpes virus vectors, and adeno-associated viral vectors) can be used for introducing a polynucleotide to a cell, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cometta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Peptide or polypeptide transfection is another method that can be used to genetically alter lymphatic endothelial progenitor cells of the disclosure and their progeny. Peptides such as Pep-1 (commercially available as Chariot), as well as other polypeptide transduction domains, can quickly and efficiently transport biologically active polypeptides, peptides, antibodies, and nucleic acids directly into cells, with an efficiency of about 60% to about 95% (Morris, M. C. et al, (2001) Nat. Biotech. 19: 1173-1176).

### Lentiviral Vectors

A method of producing a viral particle described herein will generally involve introducing a viral transfer vector and one or more additional vectors (e.g., a retroviral packaging vector) into a cell. A variety of methods suitable for production of viral vectors of the disclosure are known, such as those presented in Merten, et al., “Production of lentiviral vectors”, Mol Ther Methods Clin Dev, 3:16017 (2016) and in Nasri, et al., “Production, purification and titration of a lentivirus-based vector for gene delivery purposes”, Cytotechnology, 66:1031-1038 (2014), the disclosures of which are incorporated herein by reference in their entireties for all purposes.

In embodiments, the production of a viral particle involves introducing into a cell (i.e., a producer cell) a viral transfer vector containing a heterologous gene sequence, a packaging vector, and an envelope vector. In embodiments, the viral transfer vector contains a heterologous polynucleotide sequence containing a heterologous gene flanked by long terminal repeat (LTR) sequences, which facilitate integration of the heterologous gene sequence into the genome of a target cell. In embodiments, the transfer vector may contain a deletion in a 3′LTR to render the viral particle self-inactivating (SIN) after integration of the polynucleotide into the genome of the target cell.

The vectors may be introduced into the cell using transfection methods well known in the art. After transfection, the cell may be permitted to express viral proteins encoded by the viral transfer vector and/or the one or more additional vectors (e.g., by incubating the cell under standard conditions known in the art for inducing viral gene expression). In embodiments, the viral genes are expressed under the control of a constitutive or inducible promoter. In the latter case, viral gene expression may be selectively induced by incubating the cell under conditions suitable for activating the inducible promoter. Viral proteins produced by the cell may subsequently form a viral particle, which buds from the cell surface and can be isolated from the solution (e.g., according to methods well known in the art). When the viral particle buds from the cell surface and obtains a viral envelope containing a portion of the lipid membrane of the cell from which it budded as well as associated membrane proteins (e.g., a hemagglutinin) that were contained within the lipid membrane of the cell. During formation of the virus, a polynucleotide encoding a heterologous polypeptide may be incorporated into the viral particle. Thus, this process yields a retroviral particle that includes a polynucleotide encoding a heterologous gene (e.g., a heterologous polypeptide), where the polynucleotide sequence originated from the viral transfer vector.

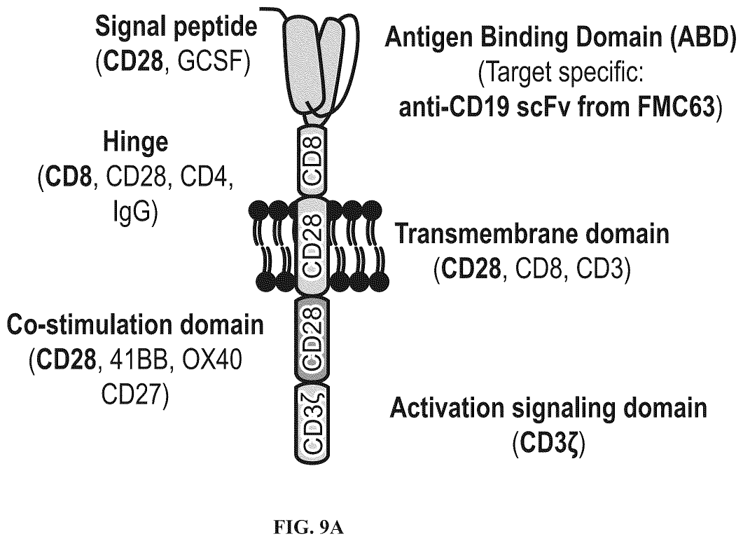

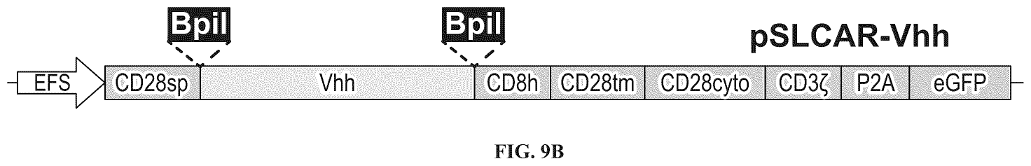

The heterologous gene may include a gene encoding a polypeptide (e.g., a VHH antibody, a chimeric antigen receptor (e.g., FIG. 9B), or a screening construct (e.g., FIG. 11)) or a gene for a noncoding RNA that is to be expressed in a target cell. In some instances, the heterologous protein ORF is positioned downstream of a Kozak sequence. In some instances, the polynucleotide of the viral transfer vector will be present in a retroviral particle produced in a cell transfected with the viral transfer vector and, optionally, one or more additional vectors (e.g., packaging vectors). In certain instances, the polynucleotide may be integrated into the genome of a cell infected with the retroviral particle. Integration of the heterologous nucleic acid into the genome of such a cell may permit the cell and its progeny to express the heterologous gene of interest. The gene of interest may be any gene known in the art. Exemplary genes of interest include, without limitation, genes encoding chimeric antigen receptors (CARs), binding moieties (e.g., VHH antibodies and antibody fragments), signaling proteins, cell surface proteins (e.g., T cell receptors), proteins involved in disease (e.g., cancers, autoimmune diseases, neurological disorders, or any other disease known in the art), or any derivative or combination thereof. In embodiments, the heterologous polypeptide is an antigen (e.g., an influenza, coronavirus, cancer, or cytomegalovirus antigen). In embodiments, the heterologous polypeptide is a therapeutic polypeptide (e.g., a chimeric antigen receptor (CAR)).

A viral transfer vector of the disclosure may be introduced into a cell (producer cell). The viral transfer vector is generally co-transfected into the cell together with one or more additional vectors (e.g., one or more packaging vectors). The one or more additional vectors may encode viral proteins and/or regulatory proteins. Co-transfection of the viral transfer vector and the one or more additional vectors enables the host cell to produce a viral particle (e.g., a lentivirus containing a polynucleotide from the lentiviral transfer vector). Retroviral particles produced by a cell as described herein may be used to infect another cell. The polynucleotide containing a heterologous gene sequence (e.g., encoding a polypeptide of interest) and/or one or more additional elements (e.g., promoters and viral elements) may be integrated into the genome of the infected cell, thereby permitting the cell and its progeny to express gene(s) originating from the viral transfer vector.

A producer cell suitable for transfection with the lentiviral transfer vector (and one or more packaging vectors) may be a eukaryotic cell, such as a mammalian cell. The host cell may originate from a cell line (e.g., an immortalized cell line). For example, the host cell may be a HEK 293 cell.

Target cell is the cell that is infected (transduced) with a viral particle containing a polynucleotide encoding a gene of interest. After transduction, the heterologous gene of interest is stably inserted into target cell genome and can be detected by molecular biology methods such as PCR and Southern blot. Transgene can be expressed in target cell and detected by flow cytometry or Western blot. In some instances, target cell is a human cell. In certain instances, the host cell is a particular cell type of interest, e.g., a primary T cell, SupT1 cell, Jurkat cell, or 293T cell.

The viral transfer vectors may include a polynucleotide molecule containing one or more of the following: a response element (e.g., an NFAT response element) and/or a promoter (e.g., a CMV, RSV, EFS, minimal, or EF1a promoter) driving expression of one or more viral sequences, long terminal repeat (LTR) regions (e.g., an R region or an U5 region), optionally flanking a heterologous gene sequence, a primer binding site (PBS), a packaging signal (psi) (e.g., a packaging signal including a major splice donor site (SD)), acPPT element, a Kozak sequence positioned upstream (e.g., immediately upstream) of a heterologous gene sequence to be transferred to a cell), a Rev-response element (RRE), a subgenomic promoter (e.g., P-EF1a), a heterologous gene (e.g., a heterologous gene encoding a CAR gene), a post-transcriptional regulatory element (e.g., a WPRE or HPRE), a polyA sequence, a selectable marker (e.g., a kanamycin resistance gene (nptII), ampicillin resistance gene, or a chloramphenicol resistance gene), and an origin of replication (e.g., a pUC origin of replication, an SV40 origin of replication, or an f1 origin of replication).

The viral transfer vector may also include elements suitable for driving expression of a heterologous protein in a cell. In certain instances, a Kozak sequence is positioned upstream of the heterologous protein open reading frame. For example, the viral transfer vector may include a promoter (e.g., a CMV, RSV, minimal, EFS, or EF1a promoter) that controls the expression of the heterologous nucleic acid. Other promoters suitable for use in the lentiviral transfer vector include, for example, constitutive promoters or tissue/cell type-specific promoters. In some instances, the lentiviral transfer vector includes a means of selectively marking a gene product (e.g., a polypeptide or RNA) encoded by at least a portion of the polynucleotide (e.g., a polynucleotide encoding a gene product of interest). For example, the viral transfer vector may include a marker gene (e.g., a gene encoding a selectable marker, such as a fluorescent protein (e.g., GFP, eGFP, mKate, YFP, RFP, dsRed, mCherry, or any derivative thereof)). The marker gene may be expressed independently of the gene product of interest. Alternatively, the marker gene may be co-expressed with the gene product of interest. For example, the marker gene may be under the control of the same or different promoter as the gene product of interest. In another example, the marker gene may be fused to the gene product of interest. The elements of the viral transfer vectors of the disclosure are, in general, in operable association with one another, to enable the transfer vectors together with one or more packaging vectors to participate in the formation of a pesudotyped viral particle in a transfected cell.

The viral transfer vectors of the disclosure may be co-transfected into a cell together with one or more additional vectors. In some instances, the one or more additional vectors may include lentiviral packaging vectors and/or envelop vectors. In certain instances, the one or more additional vectors may include an envelope vector. Generally, a packaging vector includes one or more polynucleotide sequences encoding viral proteins (e.g., gag, pol, env, tat, rev, vif, vpu, vpr, and/or nef protein, or a derivative, combination, or portion thereof). A packaging vector to be co-transfected into a cell with a viral transfer vector of the disclosure may include sequence(s) encoding one or more viral proteins not encoded by the transfer vector. For example, a viral transfer vector may be co-transfected with a first packaging vector encoding gag and pol and a second packaging vector encoding rev. Thus, co-transfection of a viral transfer vector with such packaging vector(s) may result in the introduction of all genes required for viral particle formation into the cell, thereby enabling the cell to produce viral particles that may be isolated. Further, the viral particles produced by the cell lack genes critical for viral particle formation and are, thus, incapable of self-replication. For various safety reasons, it can be advantageous to produce viral particles and are incapable of self-replication. Appropriate packaging vectors for use in the disclosure can be selected by those of skill in the art based on, for example, consideration of the features selected for a viral transfer vector of the disclosure. For examples of packaging vectors that can be used or adapted for use in the disclosure see, e.g., WO 03/064665, WO 2009/153563, U.S. Pat. No. 7,419,829, WO 2004/022761, U.S. Pat. No. 5,817,491, WO 99/41397, U.S. Pat. Nos. 6,924,123, 7,056,699, WO 99/32646, WO 98/51810, and WO 98/17815. In some instances, a packaging vector may encode a gag and/or pol protein, and may optionally include an RRE sequence (e.g., an pMDLgpRRE vector; see, e.g., Dull et al., J. Virol. 72(11):8463-8471, 1998). In certain instances, a packaging vector may encode a rev protein (e.g., a pRSV-Rev vector).

Chimeric Antigen Receptors and CAR-T cells

The disclosure provides immune cells that express chimeric antigen receptors (CARs) containing VHH domains as antigen binding domains. Embodiments of chimeric antigen receptors comprising VHH domains are shown in FIGS. 9A and 9B. Further non-limiting examples of CARs include those described in Larson and Maus, “Recent advances and discoveries in the mechanisms and functions of CAR T cells,” Nature Reviews Cancer, 21:145-161 (2021), the disclosure of which is incorporated herein by reference in its entirety for all purposes. Modification of immune cells to express a chimeric antigen receptor can enhance an immune cell's immunoreactive activity. In embodiments, the chimeric antigen receptor has an affinity for an epitope on an antigen. In some cases, the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen.

Some embodiments of the methods provided herein involve autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a neoplasia (e.g., a cancer). In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

One technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the disclosure are effector T cells or NK cells. In some embodiments, the effector T cell is a naïve CD8⁺ T cell, a cytotoxic T cell, a natural killer T (NKT) cell, a natural killer cell, a gammadelta T cell (γδ T cell), or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, the immune effector cell is any other subset of T cells.

Chimeric antigen receptors (CARs) as contemplated in the present disclosure comprise an extracellular binding domain (e.g., a VHH domain), a transmembrane domain (e.g., a transmembrane domain derived from a CD28, CD8, or CD3 polypeptide), and an intracellular domain (e.g., an intracellular domain containing a CD3ζ signaling domain, and/or a co-stimulation domain derived from a CD28, 41BB, OX40, or CD27 polypeptide). Binding of an antigen to the extracellular binding domain can activate the CAR-T cell and generate an effector response, which includes CAR-T cell proliferation, cytokine production, and other processes that lead to the death of the antigen expressing cell. In some embodiments of the present disclosure, the chimeric antigen receptor further comprises a linker (e.g., a hinge domain derived from a CD8 CD28, CD4, or IgG polypeptide). In some cases, the CAR comprises a signal peptide, such as a signal peptide derived from a CD28 or GCSF signal peptide.

In various embodiments, the CAR specifically binds any antigen that can be targeted by a chimeric antigen receptor (CAR), such as mesothelin. Further non-limiting examples of antigens that can be bound by a CAR of the present disclosure include those described in Xu, et al. “The development of CAR design for tumor CAR-T cell therapy,” Oncotarget, 9(17) doi: 10.18632/oncotarget.24179, the disclosure of which is incorporated herein by reference in its entirety for all purposes.

Chimeric antigen receptors, or any polypeptide of the present disclosure, can be delivered to an immune cell using a polynucleotide encoding the chimeric antigen receptor or polypeptide. An embodiment of a polynucleotide encoding a chimeric antigen receptor is provided in FIG. 9B. For example, immune cells obtained from a subject may be transduced with a nucleic acid vector encoding the chimeric antigen receptor. The vector may then be used to transduce recipient immune cells so that these cells will then express the chimeric antigen receptor. Efficient means of transducing immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

### Extracellular Binding Domain

The chimeric antigen receptors of the disclosure include an extracellular binding domain (e.g., a VHH domain). The extracellular binding domain of a chimeric antigen receptor contemplated herein comprises an amino acid sequence of an antibody (e.g., a VHH antibody), or an antigen binding fragment thereof, that has an affinity for a specific antigen. In some embodiments, the antigen is mesothelin.

In some embodiments the chimeric antigen receptor contains an amino acid sequence of an antibody (e.g., a VHH antibody). In some embodiments, the chimeric antigen receptor contains the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a VHH domain. In other embodiments, the antibody fragment portion of a chimeric antigen receptor is a multichain variable fragment, which can comprise more than one extracellular binding domains and therefore bind to more than one antigen simultaneously. In a multiple chain variable fragment embodiment, a hinge region may separate the different variable fragments, providing necessary spatial arrangement and flexibility.

In embodiments, the antigen-binding portion of a chimeric antigen receptor comprises complementarity determining regions (e.g., CDR1, CDR2, and CDR3 regions) that are responsible for the antibody's affinity for a particular antigen. Thus, antibodies that recognize different antigens comprise different complementarity determining regions.

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell. For example, in some embodiments, the antigen is expressed in a neoplastic cell.

### Transmembrane Domain

The chimeric antigen receptors of the disclosure include a transmembrane domain. The transmembrane domain of the chimeric antigen receptors described herein spans the CAR-T cell's or CAR NT cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell.

### Intracellular Signaling Domain

The chimeric antigen receptors of the disclosure include an intracellular signaling domain. The intracellular signaling domain is the intracellular portion of a protein expressed in a T cell or NK cell that transduces an effector function signal (e.g., an activation signal) and directs the effector cell to perform a specialized function. Effector cell activation can be induced by a number of factors, including binding of cognate antigen to the chimeric antigen receptor on the surface of effector cell and/or binding of cognate ligand to costimulatory molecules on the surface of the cell. An effector cell co-stimulatory molecule is a cognate binding partner on an immune effector cell that specifically binds with a co-stimulatory ligand, thereby mediating a co-stimulatory response by the effector cell, such as, but not limited to, proliferation. Co-stimulatory molecules include, but are not limited to, an MHC class I molecule. Activation of an effector cell leads to immune response, Such as effector cell proliferation and differentiation (see, e.g., Smith-Garvin et al., Annu. Rev. Immunol., 27:591-619, 2009). Exemplary effector cell (e.g., T cells) signaling domains are known in the art.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

### Sequencing

In embodiments, the methods of the disclosure include sequencing of a polynucleotide encoding a VHH antibody or a fragment thereof. Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference).

The sequencing of a polynucleotide can be carried out using any suitable commercially available sequencing technology (e.g., MiSeq). In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples MiSeq, RNA-seq, combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

### Recombinant Polypeptide Expression

In general, polypeptides of the disclosure (e.g., VHH antibodies) may be produced by transformation of a suitable host cell with all or part of a polypeptide-encoding nucleic acid molecule or fragment thereof in a suitable expression vehicle.

Those skilled in the field of molecular biology will understand that any of a wide variety of systems may be used to express a recombinant protein (see, e.g., FIGS. 9B and 11). The precise host cell used is not critical to the various aspects and embodiments of the disclosure. A polypeptide of the disclosure may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides (e.g., VHH antibodies or chimeric antigen receptors) of the disclosure. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the disclosure is expressed, it can be isolated, e.g., using affinity chromatography. In one example, an antibody (e.g., produced as described herein) raised against an antigen of the disclosure may be attached to a column and used to isolate the recombinant polypeptide. Lysis and fractionation of polypeptide-harboring cells prior to affinity chromatography may be performed by standard methods.

Once isolated, a recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the disclosure, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Compositions

Provided also are compositions (e.g., pharmaceutical compositions) for use in the methods of the disclosure. In embodiments, the composition is a pharmaceutical composition for use in treating a disease or disorder (e.g., a neoplasia). In some instances, a composition of the disclosure is used in a diagnostic method (e.g., to detect a marker associated with a disease). In some cases, a composition of the disclosure is used in one of the screening methods provided herein. In an embodiment, the compositions contain a cell, polynucleotide, vector, or polypeptide (e.g., a VHH antibody) provided herein. In some cases, the composition contains CAR T cells or CAR NK cells, as described herein and an acceptable carrier, excipient, or diluent.

The agents of the disclosure (e.g., polynucleotides, polypeptides, vectors, and/or cells) may be contained in any appropriate amount in any suitable carrier substance and is/are present in some cases in an amount of 0.01-95% by weight of the total weight of the composition. A pharmaceutical composition may be provided in a form that is suitable for a parenteral (e.g., subcutaneous, intravenous, intramuscular, or intraperitoneal) administration route, such that the agent, such as a vector or cell described herein, is systemically delivered.

The compositions of the present disclosure can be prepared in accordance with known techniques. See, e.g., Remington, The Science And Practice of Pharmacy (21st ed. 2005). In some embodiments, an agent of the disclosure is present in a reconstitutable dry composition (e.g., a lyophilized composition or powder). In embodiments, an agent is admixed with a suitable carrier prior to administration or storage, and in some embodiments, the composition further comprises an acceptable carrier (e.g., a pharmaceutically acceptable carrier). Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Carriers can include agents that can stabilize, optimize or otherwise alter the form, consistency, viscosity, pH, pharmacokinetics, or solubility of a composition. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Compositions of the disclosure can contain one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Compositions can also contain one or more osmotic modulating agents, i.e., a compound that modulates the osmotic properties (e.g., tonicity, osmolality, and/or osmotic pressure) of the formulation to a level that is acceptable, for example, to the blood stream and blood cells of recipient subjects. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

The skilled artisan can readily determine the number of cells and amount of optional additives, vehicles, and/or carriers in compositions and to be administered in methods of the disclosure. Of course, for any composition to be administered to an animal or human, and for any particular method of administration, it may be advantageous to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein, and the time for sequential administrations can be ascertained without undue experimentation.

In some embodiments, the composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. The pharmaceutical composition may be administered systemically.

The composition may be in the form of a solution, a suspension, an emulsion, an infusion device, or a delivery device for implantation, or it may be presented as a dry powder to be reconstituted with water or another suitable vehicle before use. Apart from the agent (e.g., CAR T cells, CAR NK cells, VHH antibodies, polynucleotides, or polypeptides provided herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition is formulated for intravenous delivery. The compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Except insofar as any conventional excipient medium is incompatible with a substance or its derivatives, such as by producing any undesirable biological effect or otherwise interacting in a deleterious manner with any other component(s) of the composition, its use is contemplated to be within the scope of this disclosure.

In some embodiments, compositions in accordance with the present disclosure can be used for treatment of any of a variety of diseases, disorders, and/or conditions.

### Treatments

The compositions, polynucleotides, cells (e.g., chimeric antigen receptor T or NK cells), and/or polypeptides (e.g., polypeptides containing a VHH antibody identified by the methods provided herein) provided herein can be used for treating a subject for a disease or disorder, such as a neoplasia (e.g., a lung cancer or an ovarian cancer). Generally, the methods provided herein include administering a therapeutically effective amount of an agent as provided herein, to a subject who is in need of, or who has been determined to be in need of, such treatment.

The methods provided herein include selecting a subject for and/or administering to a subject having or having a propensity to develop a neoplasia a treatment that includes a therapeutically effective amount of an immunotherapeutic agent, such as a CAR T cell or CAR NK cell of the disclosure.

In some instances, the methods provided herein involve administering to a subject in need thereof a VHH domain provided herein that has been conjugated to an agent (e.g., a cytotoxic agent). Antibody-drug conjugates are described, e.g., in Zolog, et al. “Antibody-drug conjugates,” Nature Reviews Drug Discovery, 12:259-260 (2013), the disclosure of which is incorporated herein by reference in its entirety for all purposes.

An effective amount of an agent can be administered in one or more administrations, applications or dosages. A therapeutically effective amount of a therapeutic compound or agent (i.e., an effective dosage) depends on the therapeutic compounds or agents selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents provided herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices may be used. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents may be selected to be within a range of circulating concentrations that include the ED50 with little or no toxicity. The dosage may vary within this range depending upon the dosage form employed and the route of administration utilized. For any agent used in the methods of the disclosure, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Such information can be used to more accurately determine useful doses in humans. Levels in plasma may be measured, for example, by high performance liquid chromatography.

Dosages and desired drug concentration of pharmaceutical compositions of the present disclosure may vary depending on the particular use envisioned. The determination of the appropriate dosage or route of administration (e.g., oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes) is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. In some embodiments, the dose amount of a CAR T cell is about, at least about, and/or no more than about 1e5 cells, 1e6 cells, 1e7 cells, 1e8 cells, 1e9 cells, 1e10 cells, 1e11 cells, 1e12 cells, 1e13 cells, 1e14 cells, 1e15 cells, or 1e16 cells. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Methods for characterizing the efficacy of a treatment for a neoplasia are well known in the art (e.g., computerized tomography (CT) scan, bone scan, magnetic resonance imaging (MRI), position emission tomography (PET) scan, ultrasound X-ray, biopsy, etc.).

### Hardware and Software

The present disclosure also relates to a computer system involved in carrying out the methods of the disclosure relating to both computations and sequencing. In the methods described herein, analyses (e.g., analyses of VHH antibody sequences) can be performed on general-purpose or specially-programmed hardware or software. One can then record the results on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations observed in the data. In aspects, software used to analyze the data can include code that applies an algorithm to the analysis of the results.

A computer system (or digital device) may be used to receive, transmit, display and/or store results, analyze the results, and/or produce a report of the results and analysis. A computer system may be understood as a logical apparatus that can read instructions from media (e.g., software) and/or network port (e.g., from the internet), which can optionally be connected to a server having fixed media. A computer system may comprise one or more of a CPU, disk drives, input devices such as keyboard and/or mouse, and a display (e.g., a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g., one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CR”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in the methods of the disclosure. Kits of the instant disclosure may include one or more containers comprising an agent provided herein, such as a polypeptide (e.g., a VHH antibody) or a polynucleotide encoding the same. In some embodiments, the kits further include instructions for use in accordance with the methods of this disclosure. In some embodiments, these instructions comprise a description of use of the agent to treat a disease or identify VHH antibodies that bind a target antigen. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein. The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the various aspects and embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the various aspects and embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Identification of VHH Antibodies Capable of Binding a Target Antigen Using a Chimeric Antigen Receptor—(CAR) Based Screen

Experiments were undertaken to develop a screen for the identification of VHH antibodies capable of binding an antigen and to use the screen for identification of VHH antibodies capable of binding to a mesothelin antigen. The screen, which is schematically summarized in FIGS. 1, 3, and 8, was a chimeric antigen receptor—(CAR) based screen involving positive and negative selection steps. The screen involved vaccinating mice capable of producing VHH antibodies (i.e., nanobodies) in response to an antigen (i.e., nanomice) with antigen presenting cells expressing a target antigen. The screen further involved using the CAR-based screen to screen the nanobodies produced by the mice in response to the target antigen to identify those VHH antibodies capable of binding the target antigen. Following the positive and negative selection steps, polynucleotide molecules encoding the VHH antibodies selected for in the screen were sequenced.

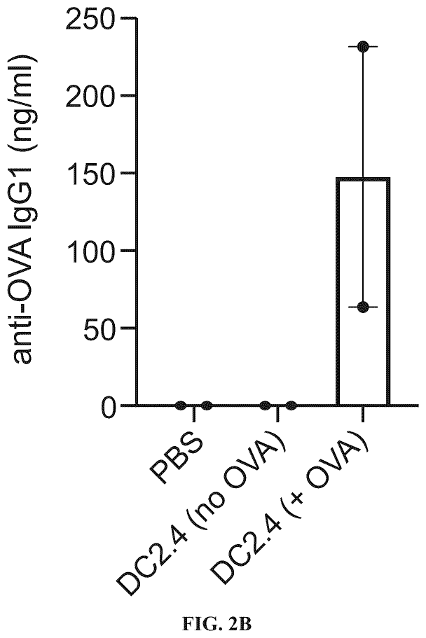

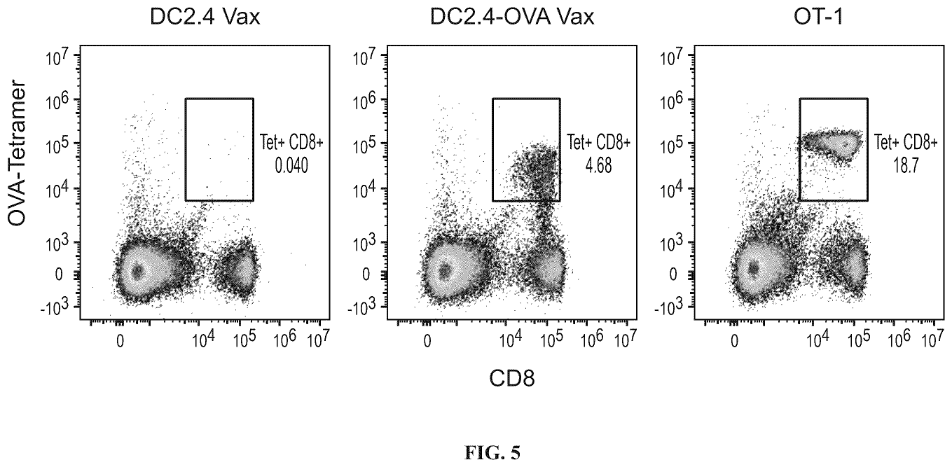

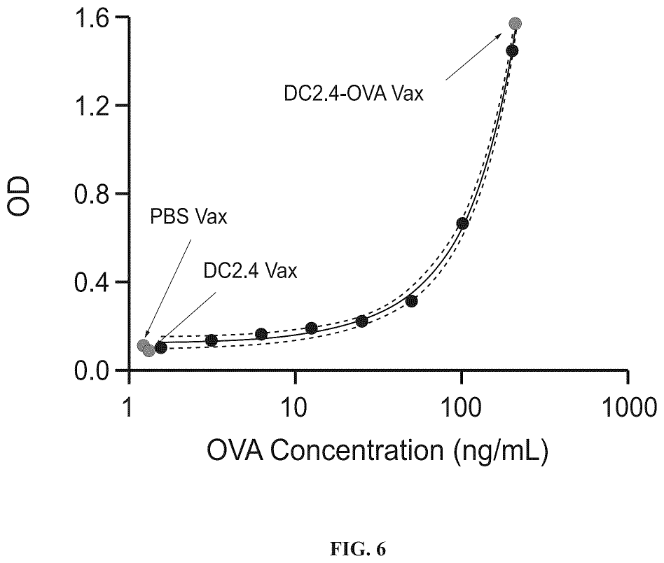

As a first step in developing the screen, experiments were undertaken to confirm that mice would produce antibodies against a target antigen presented to the cells using a live vaccine. The live vaccine contained DC2.4 cells expressing the target antigen ovalbumin (OVA). Mice were vaccinated by administering the DC2.4 cells by intraperitoneal injection (IP) and subcutaneous injection at zero (0) and two (2) weeks (FIG. 4). The DC2.4 cells were stimulated for 4 hours using CpG oligodeoxynucleotides just prior to being used to vaccinate the mice. Splenocytes and serum samples were collected from the mice at two-weeks following the second vaccination (FIG. 4). The mice produced anti-OVA immunoglobulin G1 antibodies (FIGS. 2B and 6) and T cells activated by OVA (see FIGS. 2A and 5), demonstrating that whole cell vaccination induced antigen-specific T cell expansion and a specific antibody response. Therefore, live cell vaccination was an effective way to induce mice to produce antibodies capable of binding a target antigen.

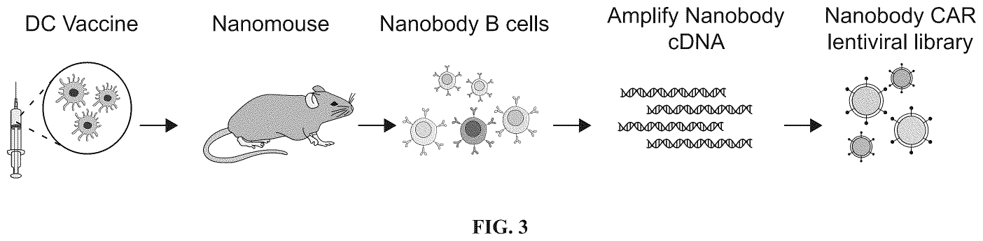

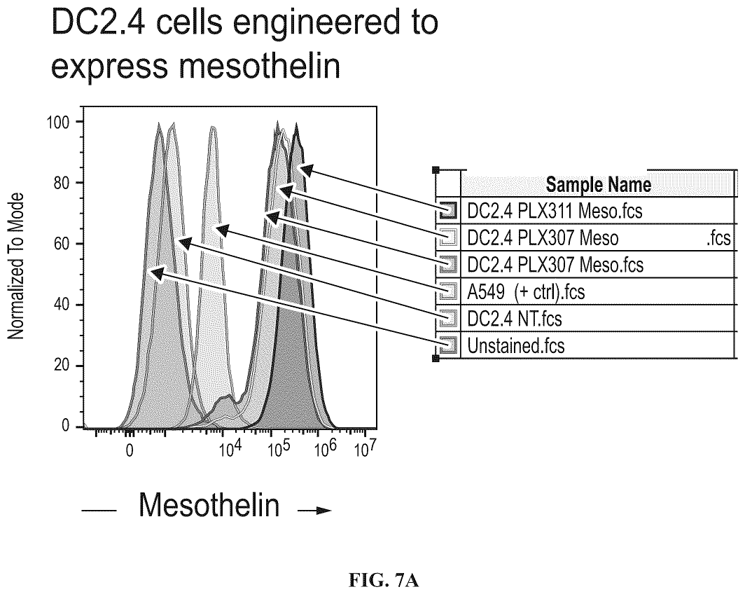

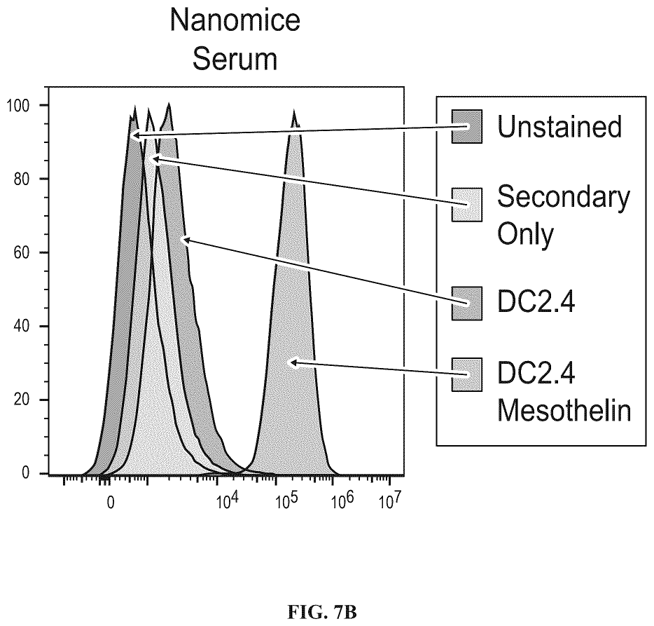

Next, experiments were undertaken to use the CAR-based screen to identify VHH antibodies capable of binding a mesothelin antigen (FIG. 3). First, to prepare a live cell vaccine to administer to mice, DC2.4 cells were transduced with expression constructs encoding mesothelin. The transduced DC2.4 cells surface-presented mesothelin antigens (FIG. 7A). The DC2.4 cells expressing mesothelin were used to vaccinate nanomice, as described above (FIG. 4). Evaluation of serum samples from the vaccinated nanomice confirmed that the mice produced anti-mesothelin VHH antibodies (FIG. 7B).

Having established that the vaccinated nanomice produced anti-mesothelin nanobodies, cDNA was prepared from nanobody-encoding mRNA from the mice and used to prepare a nanobody CAR lentiviral library (FIG. 3). The nanobody CAR lentiviral library was prepared by first amplifying VHH-encoding polynucleotides from the cDNA such that the resulting amplicons contained BpiI restriction-enzyme recognition sequences at the 5′ and 3′ ends of each VHH-encoding polynucleotide amplicon. The VHH-encoding polynucleotide amplicons were next restriction digested using BpiI and directionally cloned in-frame into the chimeric antigen receptor (CAR) expression construct shown in FIG. 9B. The resulting encoded chimeric antigen receptors (FIG. 9A) contained from N-terminus to C-terminus a CD28 signal peptide, a cloned VHH domain as an antigen-binding domain (ABD), a CD8 hinge domain, a CD28 transmembrane domain, a CD28 cytoplasmic domain, and a CD3ζ domain. The expression construct further encoded an enhanced GFP (eGFP) protein linked to the C-terminus of the CAR by a P2A self-cleaving peptide. Lentiviral vectors suitable for transduction of Jurkat cells were prepared containing the expression construct.

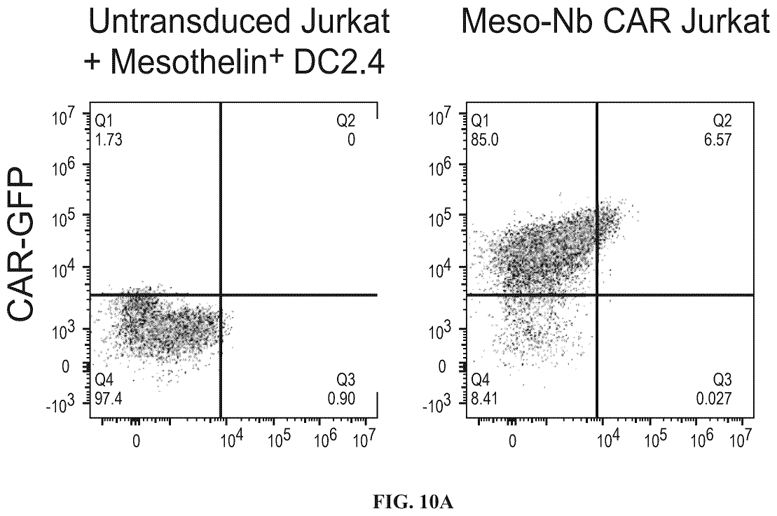

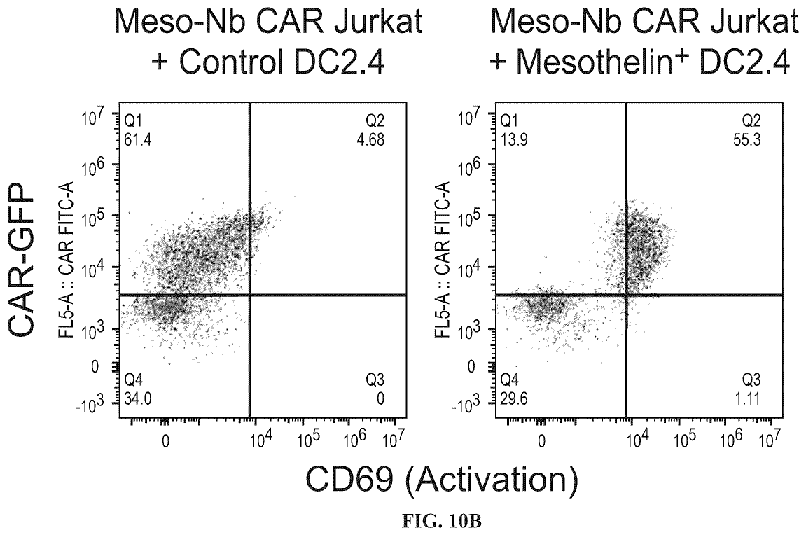

Jurkat cells were transduced using the lentiviral vectors. Jurkat cells expressing the chimeric antigen receptors were activated when co-cultured with DC2.4 cells expressing mesothelin but not when co-cultured with DC2.4 cells not expressing mesothelin (FIGS. 10A and 10B).

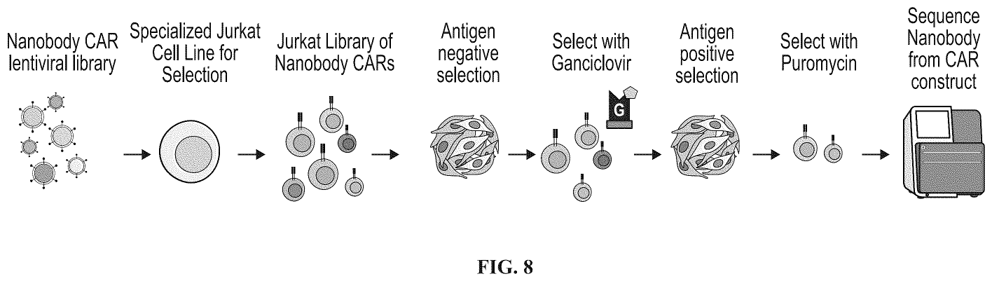

Next, Jurkat cells containing the screening construct shown in FIG. 11 were transduced using the lentiviral vectors (FIG. 8). The screening construct was a polynucleotide containing three tandem repeats of the nuclear factor of activated T cells (NFAT) response element (RE) upstream of a minimal promoter controlling transcription from a polynucleotide sequence encoding a polypeptide such that the polypeptide was selectively expressed when the antigen-binding domain of the chimeric antigen receptor expressed by the cell was bound by an antigen. The encoded polypeptide contained a puromycin resistance (PuroR) polypeptide capable of inactivating puromycin, an HSV Thymidine Kinase (HSV-TK) polypeptide capable of converting ganciclovir into a toxic product, and the fluorescent protein mKate, each linked to one another by self-cleaving peptides (i.e., P2A and T2A) (FIG. 11). The NFAT response element showed only low levels of basal activation, if any, in Jurkat cells. The puromycin resistance polypeptide (PuroR) allowed for positive selection for cells expressing chimeric antigen receptors containing a VHH domain binding a mesothelin antigen by selecting the cells with the mesothelin antigen and puromycin. The HSV thymidine kinase allowed for negative section against cells expressing chimeric antigen receptors containing a VHH domain that were activated when contacted with a non-target antigen or no antigen by selecting the cells with ganciclovir in the absence of a mesothelin antigen. The red fluorescent protein, mKate, was used to monitor construct expression.

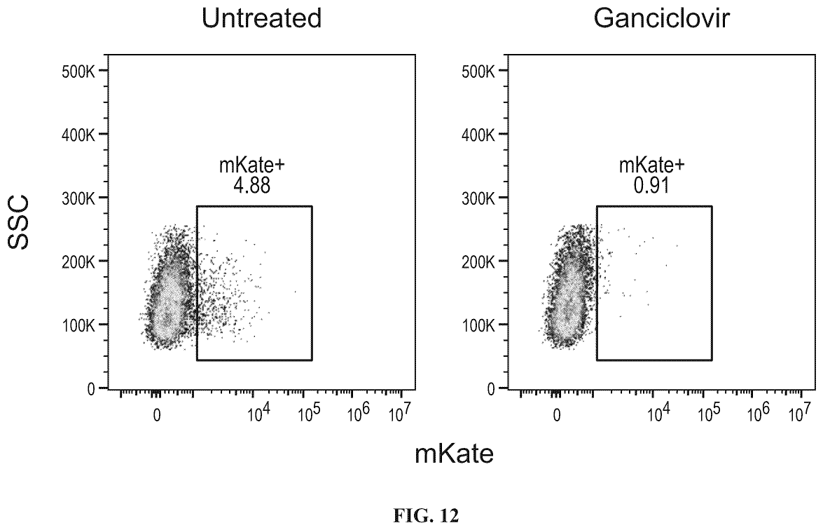

A negative selection was completed by coculturing the Jurkat cells with DC2.4 cells that did not express mesothelin and then selecting the cells with ganciclovir. Selecting the cells with ganciclovir eliminated from the cell population Jurkat cells activated in the absence of the target mesothelin antigen (FIG. 12).

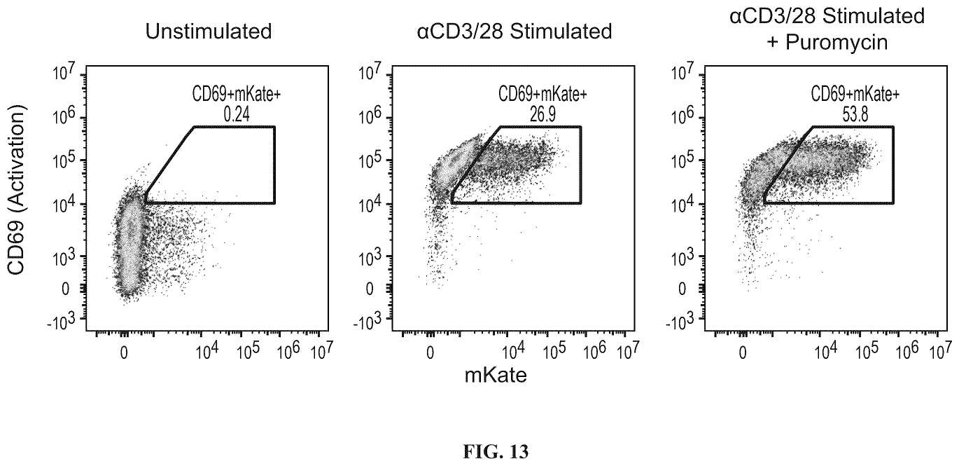

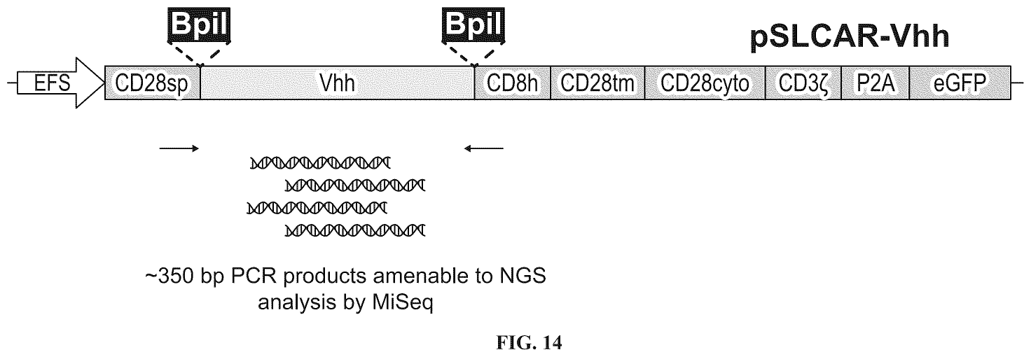

Next, the Jurkat cells that survived the negative selection were subjected to a positive selection by coculturing the Jurkat cells with DC2.4 cells that expressed mesothelin and then selecting the cells with puromycin. Selecting the cells with puromycin eliminated from the cell population Jurkat cells that failed to be activated by a mesothelin antigen displayed on the surface of the DC2.4 cells (FIG. 13).

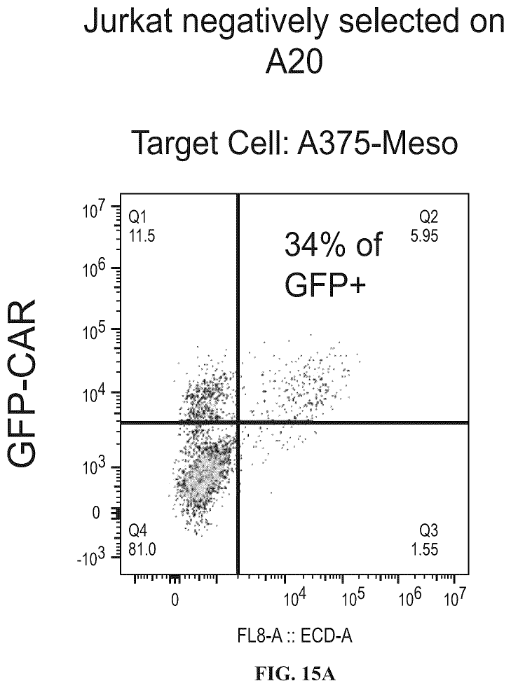

Finally, having completed the negative and positive selections to enrich the Jurkat cells for those cells expressing chimeric antigen receptors capable of selectively binding to a mesothelin antigen to yield an enriched Jurkat cell population, polynucleotides encoding VHH domains in the enriched Jurkat cell population were sequenced. The polynucleotides were sequenced by first preparing cDNA from mRNA of the Jurkat cells and then preparing amplicons about 350 bp in length that contained VHH-encoding sequences and that were amenable to next generation sequencing analysis using MiSeq (FIG. 14). The amplicons were sequenced using MiSeq and the sequences were analyzed to identify sequences of VHH antibodies capable of selectively binding to a mesothelin antigen.

### Example 2: Demonstration of the Efficacy of the Positive and Negative Selection Screens for the Identification of VHH Antibodies

Experiments were undertaken to demonstrate use of the positive and negative selections described in Example 1 (see FIGS. 1, 3, and 8) in the identification of VHH antibodies of interest. In vitro screens were carried out using a small, custom VHH “mini pool” lentiviral library containing pSLCAR chimeric antigen-receptor (CAR) vectors encoding the VHH domains listed in Table 1. Only one of the VHH domains in the library was capable of binding mesothelin. In particular, the anti-mesothelin (MSLN) VHH CAR was a known, functionally validated binder of human Mesothelin. Jurkat cells containing the screening construct shown in FIG. 11 were transduced with the mini pool lentiviral library at high >1000× coverage to yield Jurkat CAR-T cells, which were then subjected to the positive and negative selections, as described below.

TABLE 1
VHH domains used in the VHH “mini pool” library.

| VHH Domain name | Target | Affinity for Target | |
|---|---|---|---|
| N11 | mMHCII | ~single nM | |
| DC8 | mMHCII | 20 | nM |
| CD15 | mMHCII | 25 | nM |
| CD1 | mMHCII | 200 | nM |
| Meso | Mesothelin | high | |
| CD19nb1 | hCD19 | non-functional | |
| LAG-17 | GFP | 50 | nM |

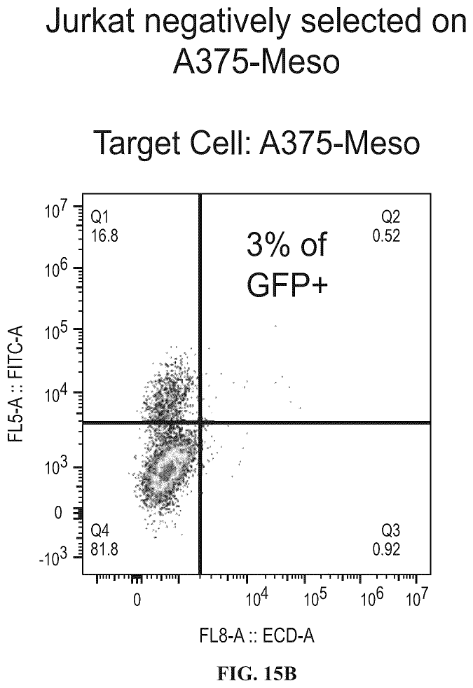

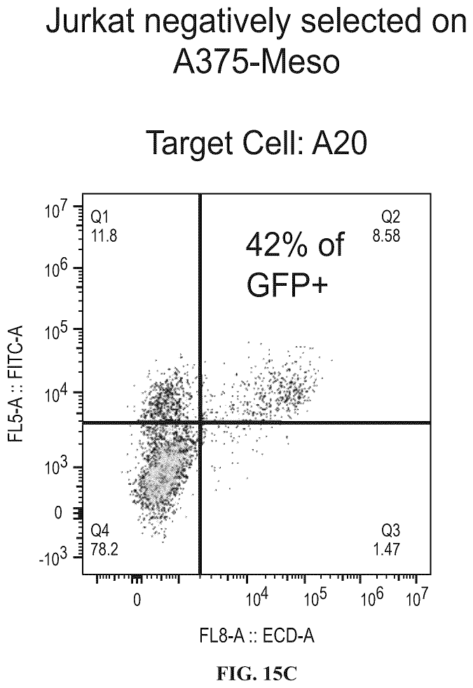

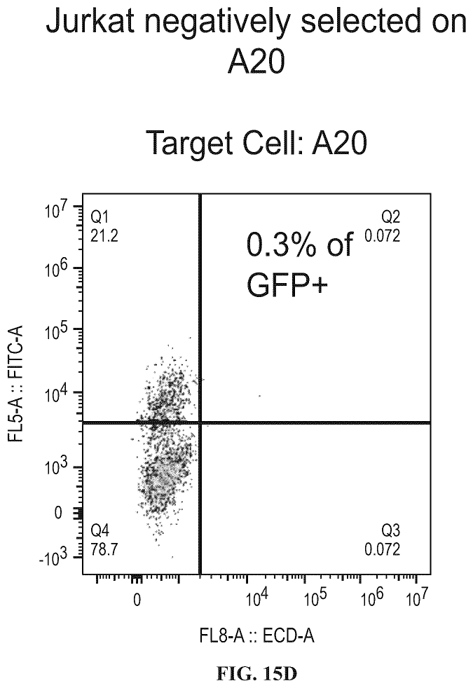

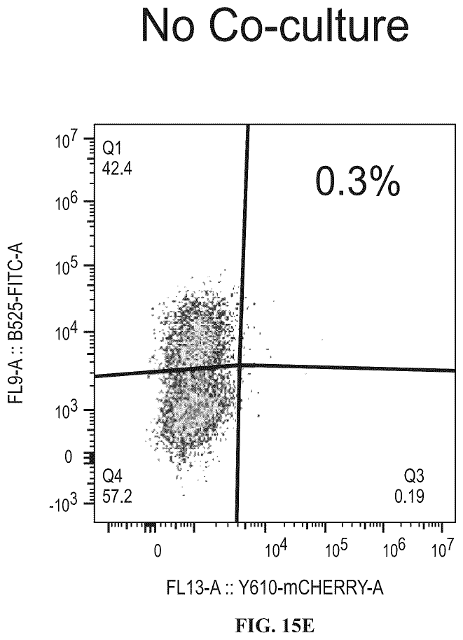

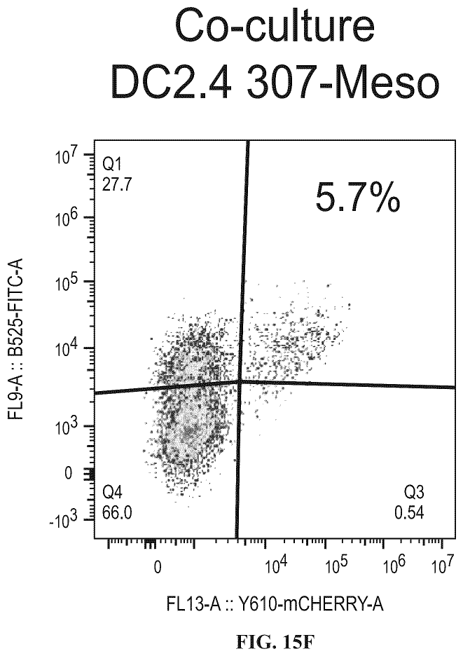

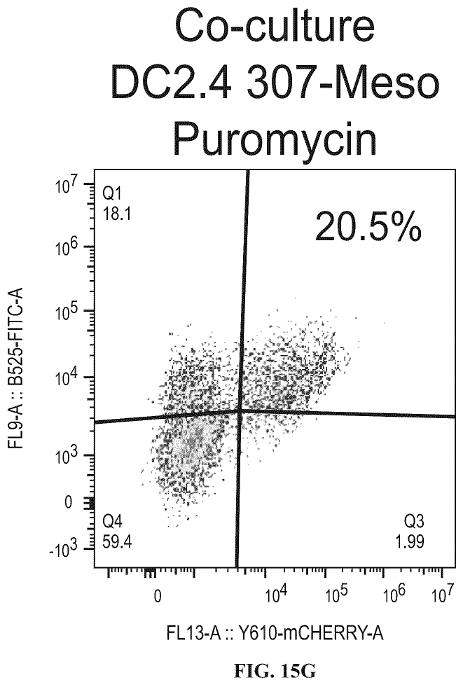

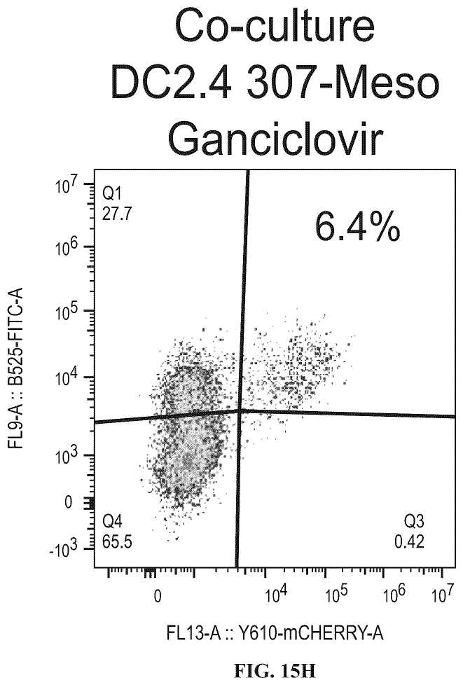

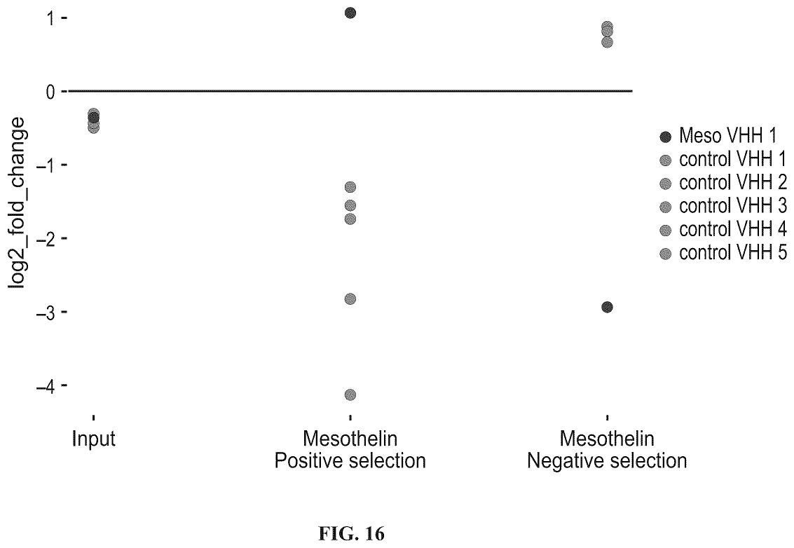

To demonstrate the effectiveness of the negative screen (i.e., a screen where activated CAR T cells are killed), the Jurkat CAR-T cells were co-cultured in ganciclovir-containing medium with either mesothelin expressing A375 cells (MSLN+ A375 cells) or with mouse major histocompatibility complex II expressing A20 cells (mMHCII+ cells). The co-culture containing the A375 cells selected for cells that were not activated by mesothelin and the co-culture containing the A20 cells selected for cells that were not activated by mMHCII. Activated cells expressed HSV Thymidine Kinase, which converted the ganciclovir to a cytotoxic compound killing the activated cells. As shown in FIG. 15A, after co-culturing the Jurkat CAR-T cells with the A20 cells (i.e., negatively selected on A20 cells), 34% of the selected Jurkat CAR-T cells were subsequently activated when co-cultured with the A375 cells, which confirmed that anti-mMHCII Jurkat CAR-T cells were successfully selected against in the negative screen carried out using the mMHCII+ A20 cells. As shown in FIG. 15B, after co-culturing the Jurkat CAR-T cells with the A375 cells (i.e., negatively selected on A375-Meso cells), only 3% of the selected Jurkat CAR-T cells were subsequently activated when co-cultured with the A375 cells again, which confirmed that anti-mesothelin Jurkat CAR-T cells were depleted in the negative screen carried out using the A375 cells, as was expected. Further, depletion of the anti-mesothelin Jurkat CAR-T cells was confirmed through next-generation sequencing (NGS) of the Jurkat CAR-T cells that survived the negative selection (i.e., the selected Jurkat CAR-T cells) (see FIG. 16). As shown in FIG. 15C, after co-culturing the Jurkat CAR-T cells with the A375 cells (i.e., negatively selected on A375-Meso cells), 42% of the selected Jurkat CAR-T cells were subsequently activated when co-cultured with A20 cells, which confirmed that anti-mMHCII Jurkat CAR-T cells successfully enriched in the negative screen carried out using the A375 cells. As shown in FIG. 15D, after co-culturing the Jurkat CAR-T cells with the A20 cells (i.e., negatively selected on A20 cells), only 0.3% of the selected Jurkat CAR-T cells were subsequently activated when co-cultured with A20 cells again, which confirmed that anti-mMHCII Jurkat CAR-T cells were depleted in the negative screen carried out using the A20 cells. These results demonstrated that the negative screen was effective in removing from a cell culture those CAR T cells activated by an antigen (e.g., mesothelin or mMHCII) presented in the negative screen.

To demonstrate the effectiveness of the positive screen (i.e., a screen where activated CAR T cells survive), the Jurkat CAR-T cells were co-cultured in puromycin-containing medium with DC2.4 cells surface expressing mesothelin from a pLX307 plasmid encoding mesothelin. As controls, the cells were not cocultured with any cells (FIG. 15E), the cells were co-cultured in the absence of puromycin or ganciclovir (FIG. 15F), or the cells were co-cultured in the presence of ganciclovir but in the absence of puromycin (FIG. 15H). Activated cells expressed a puromycin resistance gene endowing them with resistance to killing by the puromycin. It was demonstrated (FIG. 15G) that co-culturing of the Jurkat-T cells in puromycin-containing medium with the DC2.4 cells successfully enriched for anti-mesothelin Jurkat CAR-T cells. This enrichment was confirmed through next-generation sequencing (NGS) of the Jurkat CAR-T cells that survived the positive selection (i.e., the selected Jurkat CAR-T cells) (see FIG. 16). These results demonstrated that the positive screen was effective in removing from a cell culture those CAR T cells that failed to be activated by an antigen (e.g., mesothelin) presented in the positive screen.

The above experiments demonstrated that the positive and negative selections described in Example 1 (see FIGS. 1, 3, and 8) were effective for use in the identification of VHH antibodies capable of selectively binding an antigen of interest.

### Sequences

The following tables provide sequences used in the above Examples.

TABLE 2
Nucleotide sequences.

| | SEQ | | |
|---|---|---|---|
| Sequence | ID | | |
| Name | NO: | Nucleotide Sequence | Description |
| IgG1_CH2 | 3 | CCTGGTGCATGATGGGAAGT | IgG1 specific reverse primer used in |
| _GSP_R | | TC | reverse transcription reaction from |
| | | | nanomouse publication |
| TSO_with | 4 | GTCGCACGGTCCATCGCAGC | Modified template switch |
| _Mod | | AGTCrGrGrG/C3-Spacer | oligonucleotide used for reverse |
| | | | transcription |
| IgG1_CH2 | 5 | CCAGCTGAACTGGACCTCGG | IgGI specific reverse primer used in |
| _1st_R | | | first PCR reaction for amplifying |
| | | | VhhDJ cDNA |
| TSO_no_ | 6 | GTCGCACGGTCCATCGCAGC | Unmodified template switch |
| modi | | AGTC | oligonucleotide used as forward |
| | | | primer in first PCR reaction for |
| | | | amplifying VhhDJ cDNA |
| CAR_ | 7 | ttcccttcaattcaagtaac | VhhDJ Exon 1 forward primer for |
| Gibson_ | | aggaCAAGTTCAGCTTGTAG | 2nd PCR reaction with CAR adapter |
| F_Exon_1 | | AGTCAGGCG | (lowercase) for pooled gibson |
| | | | cloning into CAR vector |
| CAR_ | 8 | ttcccttcaattcaagtaac | VhhDJ Exon 2 forward primer for |
| Gibson_ | | aggaCAGGTTCAGCTGGTGG | 2nd PCR reaction with CAR adapter |
| F_Exon_2 | | AGTCC | (lowercase) for pooled gibson |
| | | | cloning into CAR vector |
| CAR_ | 9 | ttcccttcaattcaagtaac | VhhDJ Exon 3 forward primer for |
| Gibson_ | | aggaCAAGTTCAGCTTGTCG | 2nd PCR reaction with CAR adapter |
| F_Exon_3 | | AGAGTGGTG | (lowercase) for pooled gibson |
| | | | cloning into CAR vector |
| CAR_ | 10 | ttcccttcaattcaagtaac | VhhDJ Exon 4 forward primer for |
| Gibson_ | | aggaCAGGTCCAATTGGTGG | 2nd PCR reaction with CAR adapter |
| F_Exon_4 | | AATCTGGG | (lowercase) for pooled gibson |
| | | | cloning into CAR vector |
| CAR_ | 11 | ttcccttcaattcaagtaac | VhhDJ Exon 5 forward primer for |
| Gibson_ | | aggaCAAGTGCAACTTGTCG | 2nd PCR reaction with CAR adapter |
| F_Exon_5 | | AGAGCG | (lowercase) for pooled gibson |
| | | | cloning into CAR vector |
| CAR_ | 12 | ttcccttcaattcaagtaac | VhhDJ Exon 6 forward primer for |
| Gibson_ | | aggaGACGTACAACTTGTGG | 2nd PCR reaction with CAR adapter |
| F_Exon_6 | | AATCAGGTGG | (lowercase) for pooled gibson |
| | | | cloning into CAR vector |
| CAR_ | 13 | ttcccttcaattcaagtaac | VhhDJ Exon 7 forward primer for |
| Gibson_ | | aggaCAGGTTCAACTCGTTG | 2nd PCR reaction with CAR adapter |
| F_Exon_7 | | AGTCAGGC | (lowercase) for pooled gibson |
| | | | cloning into CAR vector |
| CAR_ | 14 | ttcccttcaattcaagtaac | VhhDJ Exon 8 forward primer for |
| Gibson_ | | aggaCAAGTTCAGTTGGTGG | 2nd PCR reaction with CAR adapter |
| F_Exon_8 | | AAAGTGGG | (lowercase) for pooled gibson |
| | | | cloning into CAR vector |
| CAR_ | 15 | ttcccttcaattcaagtaac | VhhDJ Exon 9 forward primer for |
| Gibson_ | | aggaCAAGTAAAACTTGAAG | 2nd PCR reaction with CAR adapter |
| F_Exon_9 | | AATCAGGTGGTGG | (lowercase) for pooled gibson |
| | | | cloning into CAR vector |
| CAR_ | 16 | ttcccttcaattcaagtaac | VhhDJ Exon 10 forward primer for |
| Gibson_ | | aggaCAAGTCCAGCTCGTCG | 2nd PCR reaction with CAR adapter |
| F_Exon_10 | | AGAGTG | (lowercase) for pooled gibson |
| | | | cloning into CAR vector |
| CAR_ | 17 | ttcccttcaattcaagtaac | VhhDJ Exon 11 forward primer for |
| Gibson_ | | aggaCAGGTCCAATTGGTGG | 2nd PCR reaction with CAR adapter |
| F_Exon_11 | | AAAGCG | (lowercase) for pooled gibson |
| | | | cloning into CAR vector |
| CAR_ | 18 | ttcccttcaattcaagtaac | VhhDJ Exon 12 forward primer for |
| Gibson_ | | aggaGAGGTCCAGGTCGTCG | 2nd PCR reaction with CAR adapter |
| F_Exon_12 | | AATCAGG | (lowercase) for pooled gibson |
| | | | cloning into CAR vector |
| CAR_ | 19 | ttcccttcaattcaagtaac | VhhDJ Exon 13 forward primer for |
| Gibson_ | | aggaGAGGTACAACTTGTGG | 2nd PCR reaction with CAR adapter |
| F_Exon_13 | | AAAGCGGTG | (lowercase) for pooled gibson |
| | | | cloning into CAR vector |
| CAR_ | 20 | ttcccttcaattcaagtaac | VhhDJ Exon 14 forward primer for |
| Gibson_ | | aggaGAAGTGCAGGTTGTCG | 2nd PCR reaction with CAR adapter |
| F_Exon_14 | | AGTCCG | (lowercase) for pooled gibson |
| | | | cloning into CAR vector |
| CAR_ | 21 | ttcccttcaattcaagtaac | VhhDJ Exon 15 forward primer for |
| Gibson_ | | aggaCAAGTAAAACTCGAAG | 2nd PCR reaction with CAR adapter |
| F_Exon_15 | | AGAGCGGG | (lowercase) for pooled gibson |
| | | | cloning into CAR vector |
| CAR_ | 22 | ttcccttcaattcaagtaac | VhhDJ Exon 16 forward primer for |
| Gibson_ | | aggaCAGCTTCAACTGGTAG | 2nd PCR reaction with CAR adapter |
| F_Exon_16 | | AGAGTGGTGG | (lowercase) for pooled gibson |
| | | | cloning into CAR vector |
| CAR_ | 23 | ttcccttcaattcaagtaac | VhhDJ Exon 17 forward primer for |
| Gibson_ | | aggaCAGGTCCAACTGGTAG | 2nd PCR reaction with CAR adapter |
| F_Exon_17 | | AAAGTGGG | (lowercase) for pooled gibson |
| | | | cloning into CAR vector |
| CAR_ | 24 | ttcccttcaattcaagtaac | VhhDJ Exon 18 forward primer for |
| Gibson_ | | aggaCAGGTCCAACTGGTAG | 2nd PCR reaction with CAR adapter |
| F_Exon_18 | | AGTCAGG | (lowercase) for pooled gibson |
| | | | cloning into CAR vector |
| CAR_ | 25 | ttcccttcaattcaagtaac | VhhDJ Exon 19 forward primer for |
| Gibson_ | | aggaGAGGTCCAACTCGTCG | 2nd PCR reaction with CAR adapter |
| F_Exon_19 | | AATCTGG | (lowercase) for pooled gibson |
| | | | cloning into CAR vector |
| CAR_ | 26 | ttcccttcaattcaagtaac | VhhDJ Exon 20 forward primer for |
| Gibson_ | | aggaCAAGTGCAATTGGTTG | 2nd PCR reaction with CAR adapter |
| F_Exon_20 | | AATCTGGTGG | (lowercase) for pooled gibson |
| | | | cloning into CAR vector |
| CAR_ | 27 | ttcccttcaattcaagtaac | VhhDJ Exon 21 forward primer for |
| Gibson_ | | aggaGAAGTCCAACTTGTAG | 2nd PCR reaction with CAR adapter |
| F_Exon_21 | | AGAGTGGG | (lowercase) for pooled gibson |
| | | | cloning into CAR vector |
| CAR_ | 28 | ttcccttcaattcaagtaac | VhhDJ Exon 22 forward primer for |
| Gibson_ | | aggaCAGGTGCAGGTTGTGG | 2nd PCR reaction with CAR adapter |
| F_Exon_22 | | AAAGC | (lowercase) for pooled gibson |
| | | | cloning into CAR vector |
| CAR_ | 29 | ttcccttcaattcaagtaac | VhhDJ Exon 23 forward primer for |
| Gibson_ | | aggaCAGGTGCAATTGGTAG | 2nd PCR reaction with CAR adapter |
| F_Exon_23 | | AGTCTGGAG | (lowercase) for pooled gibson |
| | | | cloning into CAR vector |
| CAR_ | 30 | ttcccttcaattcaagtaac | VhhDJ Exon 24 forward primer for |
| Gibson_ | | aggaCAAGTGCAGTTGGTTG | 2nd PCR reaction with CAR adapter |
| F_Exon_24 | | AGAGCG | (lowercase) for pooled gibson |
| | | | cloning into CAR vector |
| CAR_ | 31 | ttcccttcaattcaagtaac | VhhDJ Exon 25 forward primer for |
| Gibson_ | | aggaCAGGTTCAACTCGTCG | 2nd PCR reaction with CAR adapter |
| F_Exon_25 | | AATCCGG | (lowercase) for pooled gibson |
| | | | cloning into CAR vector |
| CAR_ | 32 | ttcccttcaattcaagtaac | VhhDJ Exon 26 forward primer for |
| Gibson_ | | aggaCAAGTTCAGCTTGTGG | 2nd PCR reaction with CAR adapter |
| 26 | | AGAGCG | (lowercase) for pooled gibson |
| | | | cloning into CAR vector |
| CAR_ | 33 | ttcccttcaattcaagtaac | VhhDJ Exon 27 forward primer for |
| Gibson_ | | aggaGAAGTTCAGCTTGTTG | 2nd PCR reaction with CAR adapter |
| F_Exon_27 | | AATCAGGCG | (lowercase) for pooled gibson |
| | | | cloning into CAR vector |
| CAR_ | 34 | ttcccttcaattcaagtaac | VhhDJ Exon 28 forward primer for |
| Gibson_ | | aggaCAAGTTCAACTCGTAG | 2nd PCR reaction with CAR adapter |
| F_Exon_28 | | AATCTGGCGG | (lowercase) for pooled gibson |
| | | | cloning into CAR vector |
| CAR_ | 35 | ttcccttcaattcaagtaac | VhhDJ Exon 29 forward primer for |
| Gibson_ | | aggaGAAGTTCAACTTGTCG | 2nd PCR reaction with CAR adapter |
| F_Exon_29 | | AAAGCGGAGG | (lowercase) for pooled gibson |
| | | | cloning into CAR vector |
| CAR_ | 36 | ttcccttcaattcaagtaac | VhhDJ Exon 30 forward primer for |
| Gibson_ | | aggaCAGGTCCAGCTTGTCG | 2nd PCR reaction with CAR adapter |
| F_Exon_30 | | AATCCG | (lowercase) for pooled gibson |
| | | | cloning into CAR vector |
| CAR_JH1 | 37 | ctcgcaatagttggcgctgg | VhhDJ JH1 reverse primer for 2nd |
| Gibson_R | | cgtcggcGAGGAgACGGTGA | PCR reaction with CAR adapter |
| | | CCGTGGT | (lowercase) for pooled gibson |
| | | | cloning into CAR vector |
| CAR_JH2 | 38 | ctcgcaatagttggcgctgg | VhhDJ JH2 reverse primer for 2nd |
| Gibson_R | | cgtcggcGAGGAGACTGTGA | PCR reaction with CAR adapter |
| | | GAGTGGT | (lowercase) for pooled gibson |
| | | | cloning into CAR vector |
| CAR_JH3 | 39 | ctcgcaatagttggcgctgg | VhhDJ JH3 reverse primer for 2nd |
| Gibson_R | | cgtcggcGCAGAGACAGTGA | PCR reaction with CAR adapter |
| | | CCAGAGT | (lowercase) for pooled gibson |
| | | | cloning into CAR vector |
| CAR_JH4 | 40 | ctcgcaatagttggcgctgg | VhhDJ JH4 reverse primer for 2nd |
| Gibson_R | | cgtcggcGAGGAGACGGTGA | PCR reaction with CAR adapter |
| | | CTGAGGT | (lowercase) for pooled gibson |
| | | | cloning into CAR vector |
| P5_Vhh_C | 41 | AAT GAT ACG GCG ACC | Sequencing library forward primer |
| AR_F1 | | ACC GAG ATC TAC ACT | containing illumina P5 adapter |
| | | CTT TCC CTA CAC GAC | sequence, illumina read 1 primer, |
| | | GCT CTT CCG ATC | stagger sequence and CAR plasmid |
| | | TCCATGCTCAGGCTGCTCT* | forward primer |
| | | T*G*G* C | |
| P5_Vhh_C | 42 | AAT GAT ACG GCG ACC | Sequencing library forward primer |
| AR_F2 | | ACC GAG ATC TAC ACT | containing illumina P5 adapter |
| | | CTT TCC CTA CAC GAC | sequence, illumina read 1 primer, |
| | | GCT CTT CCG ATC | stagger sequence and CAR plasmid |
| | | TCCCATGCTCAGGCTGCTCT | forward primer |
| | | *T*G*G*C | |
| P5_Vhh_C | 43 | AAT GAT ACG GCG ACC | Sequencing library forward primer |
| AR_F3 | | ACC GAG ATC TAC ACT | containing illumina P5 adapter |
| | | CTT TCC CTA CAC GAC | sequence, illumina read 1 primer, |
| | | GCT CTT CCG ATC TGC | stagger sequence and CAR plasmid |
| | | CCATGCTCAGGCTGCTCT*T | forward primer |
| | | *G*G*C | |
| P5_Vhh_C | 44 | AAT GAT ACG GCG ACC | Sequencing library forward primer |
| AR_F4 | | ACC GAG ATC TAC ACT | containing illumina P5 adapter |
| | | CTT TCC CTA CAC GAC | sequence, illumina read 1 primer, |
| | | GCT CTT CCG ATC TAG | stagger sequence and CAR plasmid |
| | | CCCATGCTCAGGCTGCTCT* | forward primer |
| | | T*G*G*C | |
| P5_Vhh_C | 45 | AAT GAT ACG GCG ACC | Sequencing library forward primer |
| AR_F5 | | ACC GAG ATC TAC ACT | containing illumina P5 adapter |
| | | CTT TCC CTA CAC GAC | sequence, illumina read 1 primer, |
| | | GCT CTT CCG ATC TCA | stagger sequence and CAR plasmid |
| | | ACCCATGCTCAGGCTGCTCT | forward primer |
| | | *T*G*G*C | |
| P5_Vhh_C | 46 | AAT GAT ACG GCG ACC | Sequencing library forward primer |
| AR_F6 | | ACC GAG ATC TAC ACT | containing illumina P5 adapter |
| | | CTT TCC CTA CAC GAC | sequence, illumina read 1 primer, |
| | | GCT CTT CCG ATC TTG | stagger sequence and CAR plasmid |
| | | CAC | forward primer |
| | | CCCATGCTCAGGCTGCTCT* | |
| | | T* G*G* C | |
| P5_Vhh_C | 47 | AAT GAT ACG GCG ACC | Sequencing library forward primer |
| AR_F7 | | ACC GAG ATC TAC ACT | containing illumina P5 adapter |
| | | CTT TCC CTA CAC GAC | sequence, illumina read 1 primer, |
| | | GCT CTT CCG ATC TAC | stagger sequence and CAR plasmid |
| | | GCA | forward primer |
| | | ACCCATGCTCAGGCTGCTCT | |
| | | *T*G*G*C | |
| P5_Vhh_C | 48 | AAT GAT ACG GCG ACC | Sequencing library forward primer |
| AR_F8 | | ACC GAG ATC TAC ACT | containing illumina P5 adapter |
| | | CTT TCC CTA CAC GAC | sequence, illumina read 1 primer, |
| | | GCT CTT CCG ATC TGA | stagger sequence and CAR plasmid |
| | | AGA CCC | forward primer |
| | | CCATGCTCAGGCTGCTCT*T | |
| | | *G*G*C | |
| P7_Vhh_C | 49 | CAAGCAGAAGACGGCATACG | Sequencing library reverse primer |
| AR_R1 | | AGATAACCGCGGGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 50 | CAAGCAGAAGACGGCATACG | Sequencing library reverse primer |
| AR_R2 | | AGATGGTTATAAGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 51 | CAAGCAGAAGACGGCATACG | Sequencing library reverse primer |
| AR_R3 | | AGATCCAAGTCCGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 52 | CAAGCAGAAGACGGCATACG | Sequencing library reverse primer |
| AR_R4 | | AGATTTGGACTTGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 53 | CAAGCAGAAGACGGCATACG | Sequencing library reverse primer |
| AR_R5 | | AGATCAGTGGATGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 54 | CAAGCAGAAGACGGCATACG | Sequencing library reverse primer |
| AR_R6 | | AGATTGACAAGCGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 55 | CAAGCAGAAGACGGCATACG | Sequencing library reverse primer |
| AR_R7 | | AGATCTAGCTTGGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 56 | CAAGCAGAAGACGGCATACG | Sequencing library reverse primer |
| AR_R8 | | AGATTCGATCCAGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 57 | CAAGCAGAAGACGGCATACG | Sequencing library reverse primer |
| AR_R9 | | AGATCCTGAACTGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 58 | CAAGCAGAAGACGGCATACG | Sequencing library reverse primer |
| AR_R10 | | AGATTTCAGGTCGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 59 | CAAGCAGAAGACGGCATACG | Sequencing library reverse primer |
| AR_R11 | | AGATAGTAGAGAGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |
| P7_Vhh_C | 60 | CAAGCAGAAGACGGCATACG | Sequencing library reverse primer |
| AR_R12 | | AGATGACGAGAGGTGACTGG | containing illumina P7 adapter |
| | | AGTTCAGACGTGTGCTCTTC | sequence, unique sample barcode |
| | | CGATCTCAATAGTTGGCGCT | sequence, illumina read 2 primer, |
| | | GGCG*T*C*G*G | and CAR plasmid reverse primer |

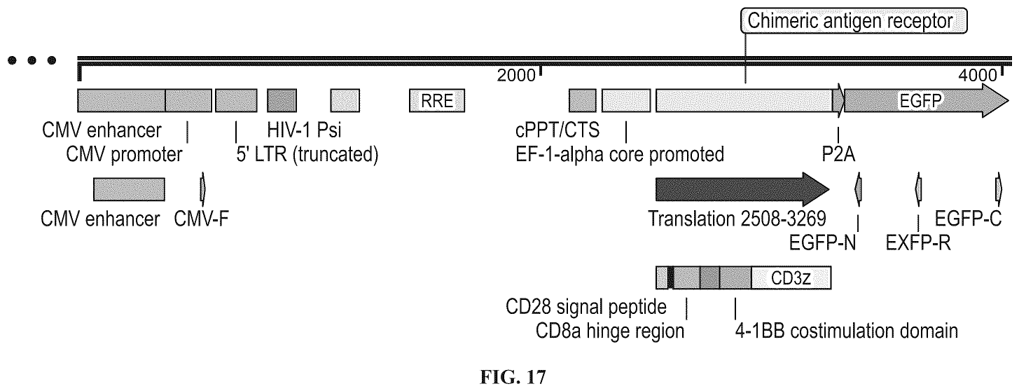

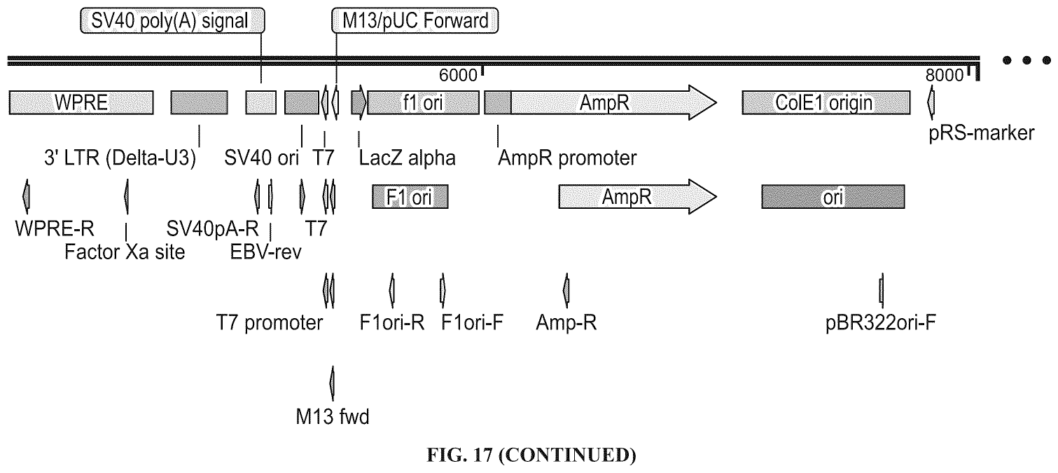

What follows is the nucleotide sequence of the chimeric antigen receptor (CARs) plasmid (pSLCAR-VHH) used for cloning of VHH domains, where the nucleotide sequence encoding a chimeric antigen receptor polypeptide linked to enhanced green fluorescent protein (eGFP) polypeptide by way of a self-cleaving peptide (P2), and prior to insertion of an anti-mesothelin VHH domain sequence, is shown in BOLD UPPERCASE TEXT. In the below sequence, sites recognized by the Bpil restriction enzyme are shown as BOLD. UPPERCASE. UNDERLINED TEXT. A map of the plasmid is provided at FIG. 17, and Tables 3A and 3B provide amino acid and polynucleotide sequences corresponding to the features labeled in the plasmid map.

(SEQ ID NO: 61)
gacattgattattgactagttattaatagtaatcaattacggggtcattagttcatagccca
tatatggagttccgcgttacataacttacggtaaatggcccgcctggctgaccgcccaacga
cccccgcccattgacgtcaataatgacgtatgttcccatagtaacgccaatagggactttcc
attgacgtcaatgggtggagtatttacggtaaactgcccacttggcagtacatcaagtgtat
catatgccaagtacgccccctattgacgtcaatgacggtaaatggcccgcctggcattatgc
ccagtacatgaccttatgggactttcctacttggcagtacatctacgtattagtcatcgcta
ttaccatggtgatgcggttttggcagtacatcaatgggcgtggatagcggtttgactcacgg
ggatttccaagtctccaccccattgacgtcaatgggagtttgttttggcaccaaaatcaacg
ggactttccaaaatgtcgtaacaactccgccccattgacgcaaatgggcggtaggcgtgtac
ggtgggaggtctatataagcagcgcgttttgcctgtactgggtctctctggttagaccagat
ctgagcctgggagctctctggctaactagggaacccactgcttaagcctcaataaagcttgc
cttgagtgcttcaagtagtgtgtgcccgtctgttgtgtgactctggtaactagagatccctc
agacccttttagtcagtgtggaaaatctctagcagtggcgcccgaacagggacttgaaagcg
aaagggaaaccagaggagctctctcgacgcaggactcggcttgctgaagcgcgcacggcaag
aggcgaggggcggcgactggtgagtacgccaaaaattttgactagcggaggctagaaggaga
gagatgggtgcgagagcgtcagtattaagcgggggagaattagatcgcgatgggaaaaaatt
cggttaaggccagggggaaagaaaaaatataaattaaaacatatagtatgggcaagcaggga
gctagaacgattcgcagttaatcctggcctgttagaaacatcagaaggctgtagacaaatac
tgggacagctacaaccatcccttcagacaggatcagaagaacttagatcattatataataca
gtagcaaccctctattgtgtgcatcaaaggatagagataaaagacaccaaggaagctttaga
caagatagaggaagagcaaaacaaaagtaagaccaccgcacagcaagcggccgctgatcttc
agacctggaggaggagatatgagggacaattggagaagtgaattatataaatataaagtagt
aaaaattgaaccattaggagtagcacccaccaaggcaaagagaagagtggtgcagagagaaa
aaagagcagtgggaataggagctttgttccttgggttcttgggagcagcaggaagcactatg
ggcgcagcgtcaatgacgctgacggtacaggccagacaattattgtctggtatagtgcagca
gcagaacaatttgctgagggctattgaggcgcaacagcatctgttgcaactcacagtctggg
gcatcaagcagctccaggcaagaatcctggctgtggaaagatacctaaaggatcaacagctc
ctggggatttggggttgctctggaaaactcatttgcaccactgctgtgccttggaatgctag
ttggagtaataaatctctggaacagatttggaatcacacgacctggatggagtgggacagag
aaattaacaattacacaagcttaatacactccttaattgaagaatcgcaaaaccagcaagaa
aagaatgaacaagaattattggaattagataaatgggcaagtttgtggaattggtttaacat
aacaaattggctgtggtatataaaattattcataatgatagtaggaggcttggtaggtttaa
gaatagtttttgctgtactttctatagtgaatagagttaggcagggatattcaccattatcg
tttcagacccacctcccaaccccgaggggaccccgggtttattacagggacagcagagatcc
actttggcgccggctcgagttttaaaagaaaaggggggattggggggtacagtgcaggggaa
agaatagtagacataatagcaacagacatacaaactaaagaattacaaaaacaaattacaaa
aattcaaaatttttcgagtggctccggtgcccgtcagtgggcagagcgcacatcgcccacag
tccccgagaagttggggggaggggtcggcaattgaaccggtgcctagagaaggtggcgcggg
gtaaactgggaaagtgatgtcgtgtactggctccgcctttttcccgagggtgggggagaacc
gtatataagtgcagtagtcgccgtgaacgttctttttcgcaacgggtttgccgccagaacac
aggtgtcgtgacgcgggatccgccaccATGCTCAGGCTGCTCTTGGCTCTCAACTTATTCCC
TTCAATTCAAGTAACAGGAGGGTCTTCCTTTTTTGAAGACCCGACGCCAGCGCCAACTATTG
CGAGTCAGCCTCTCAGTCTGCGACCTGAGGCTTGTCGACCAGCAGCCGGAGGCGCAGTGCAC
ACGAGGGGGCTGGACTTCGCCTGTGATCCTTCTAAGCCCTTTTGGGTGCTGGTGGTGGTTGG
TGGAGTCCTGGCTTGCTATAGCTTGCTAGTAACAGTGGCCTTTATTATTTTCTGGGTGAGGA
AACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACT
GGCTAGCCTGAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGACAAGAGA
CGTGGCCGGGACCCTGAGATGGGGGGAAAGCCGCAGAGAAGGAAGAACCCTCAGGAAGGCCT
GTACAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAGGAC
ACCTACGACGCCCTTCACATGCAGGCCCTGCCCCCTCGCGCTAGCGCCACGAACTTCTCTCT
GTTAAAGCAAGCAGGCGACGTGGAAGAAAACCCCGGTCCCGTGAGCAAGGGCGAGGAGCTGT
TCACCGGGGTGGTGCCCATCCTGGTCGAGCTGGACGGCGACGTAAACGGCCACAAGTTCAGC
GTGTCCGGCGAGGGCGAGGGCGATGCCACCTACGGCAAGCTGACCCTGAAGTTCATCTGCAC
CACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGACTTCAAGG
AGGACGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCGAGGA
CGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAG
CGCGATCACATGGTCCTGCTGGAGTTCGTGACCGCCGCCGGGATCACTCTCGGCATGGACGA
GCTGTACAAGtaacgcgttaagtcgacaatcaacctctggattacaaaatttgtgaaagatt
gactggtattcttaactatgttgctccttttacgctatgtggatacgctgctttaatgcctt
tgtatcatgctattgcttcccgtatggctttcattttctcctccttgtataaatcctggttg
ctgtctctttatgaggagttgtggcccgttgtcaggcaacgtggcgtggtgtgcactgtgtt
tgctgacgcaacccccactggttggggcattgccaccacctgtcagctcctttccgggactt
tcgctttccccctccctattgccacggcggaactcatcgccgcctgccttgcccgctgctgg
acaggggctcggctgttgggcactgacaattccgtggtgttgtcggggaaatcatcgtcctt
tccttggctgctcgcctgtgttgccacctggattctgcgcgggacgtccttctgctacgtcc
cttcggccctcaatccagcggaccttccttcccgcggcctgctgccggctctgcggcctctt
ccgcgtctttgccttcgccctcagacgagtcggatctccctttgggccgcctccccgcgtcg
actttaagaccaatgacttacaaggcagctgtagatcttagccactttttaaaagaaaaggg
gggactggaagggctaattcactcccaacgaagataagatctgctttttgcttgtactgggt
ctctctggttagaccagatctgagcctgggagctctctggctaactagggaacccactgctt
aagcctcaataaagcttgccttgagtgcttcaagtagtgtgtgcccgtctgttgtgtgactc
tggtaactagagatccctcagacccttttagtcagtgtggaaaatctctagcagtacgtata
gtagttcatgtcatcttattattcagtatttataacttgcaaagaaatgaatatcagagagt
gagaggaacttgtttattgcagcttataatggttacaaataaagcaatagcatcacaaattt
cacaaataaagcatttttttcactgcattctagttgtggtttgtccaaactcatcaatgtat
cttatcatgtctggctctagctatcccgcccctaactccgcccatcccgcccctaactccgc
ccagttccgcccattctccgccccatggctgactaattttttttatttatgcagaggccgag
gccgcctcggcctctgagctattccagaagtagtgaggaggcttttttggaggcctagggac
gtacccaattcgccctatagtgagtcgtattacgcgcgctcactggccgtcgttttacaacg
tcgtgactgggaaaaccctggcgttacccaacttaatcgccttgcagcacatccccctttcg
ccagctggcgtaatagcgaagaggcccgcaccgatcgcccttcccaacagttgcgcagcctg
aatggcgaatgggacgcgccctgtagcggcgcattaagcgcggcgggtgtggtggttacgcg
cagcgtgaccgctacacttgccagcgccctagcgcccgctcctttcgctttcttcccttcct
ttctcgccacgttcgccggctttccccgtcaagctctaaatcgggggctccctttagggttc
cgatttagtgctttacggcacctcgaccccaaaaaacttgattagggtgatggttcacgtag
tgggccatcgccctgatagacggtttttcgccctttgacgttggagtccacgttctttaata
gtggactcttgttccaaactggaacaacactcaaccctatctcggtctattcttttgattta
taagggattttgccgatttcggcctattggttaaaaaatgagctgatttaacaaaaatttaa
cgcgaattttaacaaaatattaacgcttacaatttaggtggcacttttcggggaaatgtgcg
cggaacccctatttgtttatttttctaaatacattcaaatatgtatccgctcatgagacaat
aaccctgataaatgcttcaataatattgaaaaaggaagagtatgagtattcaacatttccgt
gtcgcccttattcccttttttgcggcattttgccttcctgtttttgctcacccagaaacgct
ggtgaaagtaaaagatgctgaagatcagttgggtgcacgagtgggttacatcgaactggatc
tcaacagcggtaagatccttgagagttttcgccccgaagaacgttttccaatgatgagcact
tttaaagttctgctatgtggcgcggtattatcccgtattgacgccgggcaagagcaactcgg
tcgccgcatacactattctcagaatgacttggttgagtactcaccagtcacagaaaagcatc
ttacggatggcatgacagtaagagaattatgcagtgctgccataaccatgagtgataacact
gcggccaacttacttctgacaacgatcggaggaccgaaggagctaaccgcttttttgcacaa
catgggggatcatgtaactcgccttgatcgttgggaaccggagctgaatgaagccataccaa
acgacgagcgtgacaccacgatgcctgtagcaatggcaacaacgttgcgcaaactattaact
ggcgaactacttactctagcttcccggcaacaattaatagactggatggaggcggataaagt
tgcaggaccacttctgcgctcggcccttccggctggctggtttattgctgataaatctggag
ccggtgagcgtgggtctcgcggtatcattgcagcactggggccagatggtaagccctcccgt
atcgtagttatctacacgacggggagtcaggcaactatggatgaacgaaatagacagatcgc
tgagataggtgcctcactgattaagcattggtaactgtcagaccaagtttactcatatatac
tttagattgatttaaaacttcatttttaatttaaaaggatctaggtgaagatcctttttgat
aatctcatgaccaaaatcccttaacgtgagttttcgttccactgagcgtcagaccccgtaga
aaagatcaaaggatcttcttgagatcctttttttctgcgcgtaatctgctgcttgcaaacaa
aaaaaccaccgctaccagcggtggtttgtttgccggatcaagagctaccaactctttttccg
aaggtaactggcttcagcagagcgcagataccaaatactgttcttctagtgtagccgtagtt
aggccaccacttcaagaactctgtagcaccgcctacatacctcgctctgctaatcctgttac
cagtggctgctgccagtggcgataagtcgtgtcttaccgggttggactcaagacgatagtta
ccggataaggcgcagcggtcgggctgaacggggggttcgtgcacacagcccagcttggagcg
aacgacctacaccgaactgagatacctacagcgtgagctatgagaaagcgccacgcttcccg
aagggagaaaggcggacaggtatccggtaagcggcagggtcggaacaggagagcgcacgagg
gagcttccagggggaaacgcctggtatctttatagtcctgtcgggtttcgccacctctgact
tgagcgtcgatttttgtgatgctcgtcaggggggcggagcctatggaaaaacgccagcaacg
cggcctttttacggttcctggccttttgctggccttttgctcacatgtgtcgacggatcggg
agatctcccgatcccctatggtgcactctcagtacaatctgctctgatgccgcatagttaag
ccagtatctgctccctgcttgtgtgttggaggtcgctgagtagtgcgcgagcaaaatttaag
ctacaacaaggcaaggcttgaccgacaattgcatgaagaatctgcttagggttaggcgtttt
gcgctgcttcgcgatgtacgggccagatatacgcgtt

What follows is the amino acid sequence encoded by the BOLD ALL CAPS sequence shown above, which corresponds to a CAR sequence lacking an antigen binding domain (e.g., a VHH domain) that is linked to enhanced green fluorescent protein (eGFP) polypeptide by way of a self-cleaving peptide (P2A).

(SEQ ID NO: 62)
MLRLLLALNLFPSIQVTGGSSFFEDPTPAPTIASQPLSLRPEACRP
AAGGAVHTRGLDFACDPSKPFWVLVVVGGVLACYSLLVTVAFIIF
WVRKRGRKKLLYIFKQPFMRPVQTTQEEDGCSCRFPEEEEGGCEL
ASLRVKFSRSADAPAYQQGQNQLYNELNLGRREEYDVLDKRRGRD
PEMGGKPQRRKNPQEGLYNELQKDKMAEAYSEIGMKGERRRGKGH
DGLYQGLSTATKDTYDALHMQALPPRASATNFSLLKQAGDVEENP
GPVSKGEELFTGVVPILVELDGDVNGHKFSVSGEGEGDATYGKLT
LKFICTTGKLPVPWPTLVTTLTYGVQCFSRYPDHMKQHDFFKSAM
PEGYVQERTIFFKDDGNYKTRAEVKFEGDTLVNRIELKGIDFKED
GNILGHKLEYNYNSHNVYIMADKQKNGIKVNFKIRHNIEDGSVQL
ADHYQQNTPIGDGPVLLPDNHYLSTQSALSKDPNEKRDHMVLLEF
VTAAGITLGMDELYK

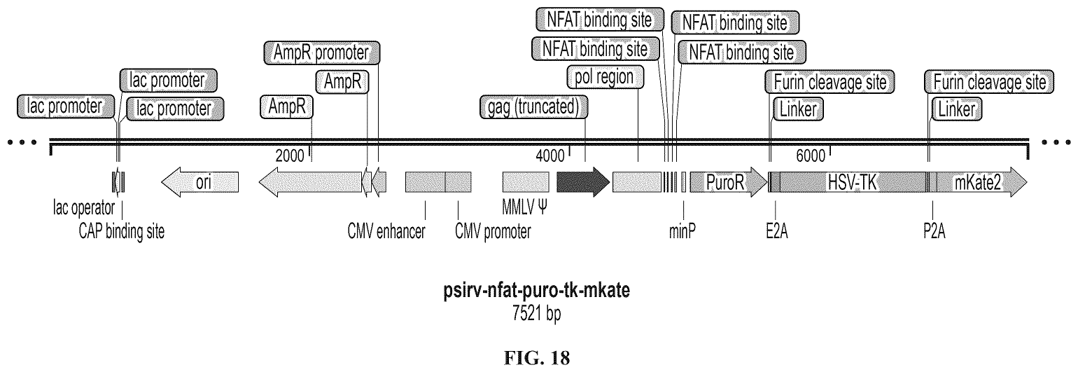

What follows is the nucleotide sequence of the plasmid encoding the screening construct used in the Examples. A map of the plasmid is provided at FIG. 18, and Tables 3A and 3B provide amino acid and polynucleotide sequences corresponding to the features labeled in the plasmid map.

(SEQ ID NO: 63)
ggccgccagcacagtggtcgatcgacgataaaataaaagattttatttagtctccagaaaaa
ggggggaatgaaagaccccacctgtaggtttggcaagctagcttaagtaacgccattttgca
aggcatggaaaaatacataactgagaatagaaaagttcagatcaaggtcaggaacagatgga
acagggtcgcgtcccgcaataaaagagcccacaacccctcactcggggcgccagtcctccga
ttgactgagtcgcccgggtacccgtgtatccaataaaccctcttgcagttgcatccgacttg
tggtctcgctgttccttgggagggtctcctctgagtgattgactacccgtcagcgggggtct
ttcacatgcagcatgtatcaaaattaatttggttttttttcttaagtatttacattaaatgg
ccatagtagttcattatggacagcgcagaaagagctggggagaattgtgaaattgttatccg
ctcacaattccacacaacatacgagccggaagcataaagtgtaaagcctggggtgcctaatg
agtgagctaactcacattaattgcgttgcgctcactgcccgctttccagtcgggaaacctgt
cgtgccagctgcattaatgaatcggccaacgcgcggggagaggcggtttgcgtattgggcgc
tcttccgcttcctcgctcactgactcgctgcgctcggtcgttcggctgcggcgagcggtatc
agctcactcaaaggcggtaatacggttatccacagaatcaggggataacgcaggaaagaaca
tgtgagcaaaaggccagcaaaaggccaggaaccgtaaaaaggccgcgttgctggcgtttttc
cataggctccgcccccctgacgagcatcacaaaaatcgacgctcaagtcagaggtggcgaaa
cccgacaggactataaagataccaggcgtttccccctggaagctccctcgtgcgctctcctg
ttccgaccctgccgcttaccggatacctgtccgcctttctcccttcgggaagcgtggcgctt
tctcatagctcacgctgtaggtatctcagttcggtgtaggtcgttcgctccaagctgggctg
tgtgcacgaaccccccgttcagcccgaccgctgcgccttatccggtaactatcgtcttgagt
ccaacccggtaagacacgacttatcgccactggcagcagccactggtaacaggattagcaga
gcgaggtatgtaggcggtgctacagagttcttgaagtggtggcctaactacggctacactag
aagaacagtatttggtatctgcgctctgctgaagccagttaccttcggaaaaagagttggta
gctcttgatccggcaaacaaaccaccgctggtagcggtggtttttttgtttgcaagcagcag
attacgcgcagaaaaaaaggatctcaagaagatcctttgatcttttctacggggtctgacgc
tcagtggaacgaaaactcacgttaagggattttggtcatgagattatcaaaaaggatcttca
cctagatccttttgcggccggccgcaaatcaatctaaagtatatatgagtaaacttggtctg
acagttaccaatgcttaatcagtgaggcacctatctcagcgatctgtctatttcgttcatcc
atagttgcctgactccccgtcgtgtagataactacgatacgggagggcttaccatctggccc
cagtgctgcaatgataccgcgagacccacgctcaccggctccagatttatcagcaataaacc
agccagccggaagggccgagcgcagaagtggtcctgcaactttatccgcctccatccagtct
attaattgttgccgggaagctagagtaagtagttcgccagttaatagtttgcgcaacgttgt
tgccattgctacaggcatcgtggtgtcacgctcgtcgtttggtatggcttcattcagctccg
gttcccaacgatcaaggcgagttacatgatcccccatgttgtgcaaaaaagcggttagctcc
ttcggtcctccgatcgttgtcagaagtaagttggccgcagtgttatcactcatggttatggc
agcactgcataattctcttactgtcatgccatccgtaagatgcttttctgtgactggtgagt
actcaaccaagtcattctgagaatagtgtatgcggcgaccgagttgctcttgcccggcgtca
atacgggataataccgcgccacatagcagaactttaaaagtgctcatcattggaaaacgttc
ttcggggcgaaaactctcaaggatcttaccgctgttgagatccagttcgatgtaacccactc
gtgcacccaactgatcttcagcatcttttactttcaccagcgtttctgggtgagcaaaaaca
ggaaggcaaaatgccgcaaaaaagggaataagggcgacacggaaatgttgaatactcatact
cttcctttttcaatattattgaagcatttatcagggttattgtctcatgagcggatacatat
ttgaatgtatttagaaaaataaacaaataggggttccgcgcacatttccccgaaaagtgcca
ccagctttgctcttaggagtttcctaatacatcccaaactcaaatatataaagcatttgact
tgttctatgccctagttattaatagtaatcaattacggggtcattagttcatagcccatata
tggagttccgcgttacataacttacggtaaatggcccgcctggctgaccgcccaacgacccc
cgcccattgacgtcaataatgacgtatgttcccatagtaacgccaatagggactttccattg
acgtcaatgggtggagtatttacggtaaactgcccacttggcagtacatcaagtgtatcata
tgccaagtacgccccctattgacgtcaatgacggtaaatggcccgcctggcattatgcccag
tacatgaccttatgggactttcctacttggcagtacatctacgtattagtcatcgctattac
catggtgatgcggttttggcagtacatcaatgggcgtggatagcggtttgactcacggggat
ttccaagtctccaccccattgacgtcaatgggagtttgttttggcaccaaaatcaacgggac
tttccaaaatgtcgtaacaactccgccccattgacgcaaatgggcggtaggcgtgtacggtg
ggaggtctatataagcagagctcaataaaagagcccacaacccctcactcggcgcgccagtc
ctccgattgactgagtcgcccgggtacccgtgtatccaataaaccctcttgcagttgcatcc
gacttgtggtctcgctgttccttgggagggtctcctctgagtgattgactacccgtcagcgg
gggtctttcatttgggggctcgtccgggatcgggagacccctgcccagggaccaccgaccca
ccaccgggaggtaagctggccagcaacttatctgtgtctgtccgattgtctagtgtctatga
ctgattttatgcgcctgcgtcggtactagttagctaactagctctgtatctggcggacccgt
ggtggaactgacgagttcggaacacccggccgcaaccctgggagacgtcccagggacttcgg
gggccgtttttgtggcccgacctgagtccaaaaatcccgatcgttttggactctttggtgca
ccccccttagaggagggatatgtggttctggtaggagacgagaacctaaaacagttcccgcc
tccgtctgaatttttgctttcggtttgggaccgaagccgcgccgcgcgtcttgtctgctgca
gcatcgttctgtgttgtctctgtctgactgtgtttctgtatttgtctgaaaatatgggcccg
ggccagactgttaccactcccttaagtttgaccttaggtcactggaaagatgtcgagcggat
cgctcacaaccagtcggtagatgtcaagaagagacgttgggttaccttctgctctgcagaat
ggccaacctttaacgtcggatggccgcgagacggcacctttaaccgagacctcatcacccag
gttaagatcaaggtcttttcacctggcccgcatggacacccagaccaggtcccctacatcgt
gacctgggaagccttggcttttgacccccctccctgggtcaagccctttgtacaccctaagc
ctccgcctcctcttcctccatccgccccgtctctcccccttgaacctcctcgttcgaccccg
cctcgatcctccctttatccagccctcactccttctctaggcgcccccatatggccatatga
gatcttatatggggcacccccgccccttgtaaacttccctgaccctgacatgacaagagtta
ctaacagcccctctctccaagctcacttacaggctctctacttagtccagcacgaagtctgg
agacctctggcggcagcctaccaagaacaactggaccgaccggtggtacctcacccttaccg
agtcggcgacacagtgtgggtccgccgacaccagactaagaacctagaacctcgctggaaag
gaccttacacagtcctgctgaccacccccaccgccctcaaagtagacggcatcgcagcttgg
atacacgccgcccacgtgaaggctgccgaccccgggggtggaccatcctctagactgccgga
tccaagctggaggaaaaactgtttcatacagaaggcgtggaggaaaaactgtttcatacaga
aggcgtggaggaaaaactgtttcatacagaaggcgtggaggaaaaactgtttcatacagaag
gcgtcgcgaattcgcggagactctagagggtatataatggaagctcgatttccagcttggca
ttccggtactgttggtaaacaccaagctatgaccgagtacaagcccacggtgcgcctcgcca
cccgcgacgacgtccccagggccgtacgcaccctcgccgccgcgttcgccgactaccccgcc
acgcgccacaccgtcgatccggaccgccacatcgagcgggtcaccgagctgcaagaactctt
cctcacgcgcgtcgggctcgacatcggcaaggtgtgggtcgcggacgacggcgccgccgtgg
cggtctggaccacgccggagagcgtcgaagcgggggcggtgttcgccgagatcggcccgcgc
atggccgagttgagcggttcccggctggccgcgcagcaacagatggaaggcctcctggcgcc
gcaccggcccaaggagcccgcgtggttcctggccaccgtcggcgtttcgcccgaccaccagg
gcaagggtctgggcagcgccgtcgtgctccccggagtggaggcggccgagcgcgccggggtg
cccgccttcctggagacctccgcgccccgcaacctccccttctacgagcggctcggcttcac
cgtcaccgccgacgtcgaggtgcccgaaggaccgcgcacctggtgcatgacccgcaagcccg
gtgccgctagccgggccaagcggtccggatccggacagtgcaccaactatgcgctgctgaaa
ctggcgggcgatgtggaaagcaacccgggcccgatggcttcgtacccctgccatcaacacgc
gtctgcgttcgaccaggctgcgcgttctcgcggccatagcaaccgacgtacggcgttgcgcc
ctcgccggcagcaagaagccacggaagtccgcctggagcagaaaatgcccacgctactgcgg
gtttatatagacggtcctcacgggatggggaaaaccaccaccacgcaactgctggtggccct
gggttcgcgcgacgatatcgtctacgtacccgagccgatgacttactggcaggtgctggggg
cttccgagacaatcgcgaacatctacaccacacaacaccgcctcgaccagggtgagatatcg
gccggggacgcggcggtggtaatgacaagcgcccagataacaatgggcatgccttatgccgt
gaccgacgccgttctggctcctcatatcgggggggaggctgggagctcacatgccccgcccc
cggccctcaccctcatcttcgaccgccatcccatcgccgccctcctgtgctacccggccgcg
cgataccttatgggcagcatgaccccccaggccgtgctggcgttcgtggccctcatcccgcc
gaccttgcccggcacaaacatcgtgttgggggcccttccggaggacagacacatcgaccgcc
tggccaaacgccagcgccccggcgagcggcttgacctggctatgctggccgcgattcgccgc
gtttacgggctgcttgccaatacggtgcggtatctgcagggcggcgggtcgtggcgggagga
ttggggacagctttcggggacggccgtgccgccccagggtgccgagccccagagcaacgcgg
gcccacgaccccatatcggggacacgttatttaccctgtttcgggcccccgagttgctggcc
cccaacggcgacctgtacaacgtgtttgcctgggccttggacgtcttggccaaacgcctccg
tcccatgcacgtctttatcctggattacgaccaatcgcccgccggctgccgggacgccctgc
tgcaacttacctccgggatggtccagacccacgtcaccacccccggctccataccgacgatc
tgcgacctggcgcgcacgtttgcccgggagatgggggaggctaaccgcgccaagcgctcggg
ttcgggtgccaccaacttcagcctgctgaagcaggccggcgacgtggaggagaaccccggcc
ccatggtgagcgagctgattaaggagaacatgcacatgaagctgtacatggagggcaccgtg
aacaaccaccacttcaagtgcacatccgagggcgaaggcaagccctacgagggcacccagac
catgagaatcaaggcggtcgagggcggccctctccccttcgccttcgacatcctggctacca
gcttcatgtacggcagcaaaaccttcatcaaccacacccagggcatccccgacttctttaag
cagtccttccccgagggcttcacatgggagagagtcaccacatacgaagacgggggcgtgct
gaccgctacccaggacaccagcctccaggacggctgcctcatctacaacgtcaagatcagag
gggtgaacttcccatccaacggccctgtgatgcagaagaaaacactcggctgggaggcctcc
accgagaccctgtaccccgctgacggcggcctggaaggcagagccgacatggccctgaagct
cgtgggcgggggccacctgatctgcaacttgaagaccacatacagatccaagaaacccgcta
agaacctcaagatgcccggcgtctactatgtggacagaagactggaaagaatcaaggaggcc
gacaaagagacctacgtcgagcagcacgaggtggctgtggccagatactgcgacctccctag
caaactggggcacagataa

TABLE 3A
Nucleotide sequences for plasmid features.

| | SEQ ID | |
|---|---|---|
| Feature Name | NO: | Nucleotide Sequence |
| CMV Enhancer | 64 | GACATTGATTATTGACTAGTTATTAATAGTAATCAATTA |
| | | CGGGGTCATTAGTTCATAGCCCATATATGGAGTTCCGCG |
| | | TTACATAACTTACGGTAAATGGCCCGCCTGGCTGACCGC |
| | | CCAACGACCCCCGCCCATTGACGTCAATAATGACGTATG |
| | | TTCCCATAGTAACGCCAATAGGGACTTTCCATTGACGTC |
| | | AATGGGTGGAGTATTTACGGTAAACTGCCCACTTGGCAG |
| | | TACATCAAGTGTATCATATGCCAAGTACGCCCCCTATTG |
| | | ACGTCAATGACGGTAAATGGCCCGCCTGGCATTATGCCC |
| | | AGTACATGACCTTATGGGACTTTCCTACTTGGCAGTACA |
| | | TCTACGTATTAGTCATCGCTATTACCATG; or |
| | 65 | CGTTACATAACTTACGGTAAATGGCCCGCCTGGCTGACC |
| | | GCCCAACGACCCCCGCCCATTGACGTCAATAATGACGTA |
| | | TGTTCCCATAGTAACGCCAATAGGGACTTTCCATTGACG |
| | | TCAATGGGTGGAGTATTTACGGTAAACTGCCCACTTGGC |
| | | AGTACATCAAGTGTATCATATGCCAAGTACGCCCCCTAT |
| | | TGACGTCAATGACGGTAAATGGCCCGCCTGGCATTATGC |
| | | CCAGTACATGACCTTATGGGACTTTCCTACTTGGCAGTA |
| | | CATCTACGTATTAGTCATCGCTATTACCATG |
| CMV promoter | 66 | TGATGCGGTTTTGGCAGTACATCAATGGGCGTGGATAGC |
| | | GGTTTGACTCACGGGGATTTCCAAGTCTCCACCCCATTG |
| | | ACGTCAATGGGAGTTTGTTTTGGCACCAAAATCAACGGG |
| | | ACTTTCCAAAATGTCGTAACAACTCCGCCCCATTGACGC |
| | | AAATGGGCGGTAGGCGTGTACGGTGGGAGGTCTATATAA |
| | | GCAG |
| CMF-F | 67 | CGCAAATGGGCGGTAGGCGTG |
| 5′ LTR | 68 | GGGTCTCTCTGGTTAGACCAGATCTGAGCCTGGGAGCTC |
| (truncated) | | TCTGGCTAACTAGGGAACCCACTGCTTAAGCCTCAATAA |
| | | AGCTTGCCTTGAGTGCTTCAAGTAGTGTGTGCCCGTCTG |
| | | TTGTGTGACTCTGGTAACTAGAGATCCCTCAGACCCTTT |
| | | TAGTCAGTGTGGAAAATCTCTAGCA |
| HIV-1 Psi | 69 | CTCTCTCGACGCAGGACTCGGCTTGCTGAAGCGCGCACG |
| | | GCAAGAGGCGAGGGGCGGCGACTGGTGAGTACGCCAAAA |
| | | ATTTTGACTAGCGGAGGCTAGAAGGAGAGAGATGGGTGC |
| | | GAGAGCGTC |
| RRE | 70 | AGGAGCTTTGTTCCTTGGGTTCTTGGGAGCAGCAGGAAG |
| | | CACTATGGGCGCAGCGTCAATGACGCTGACGGTACAGGC |
| | | CAGACAATTATTGTCTGGTATAGTGCAGCAGCAGAACAA |
| | | TTTGCTGAGGGCTATTGAGGCGCAACAGCATCTGTTGCA |
| | | ACTCACAGTCTGGGGCATCAAGCAGCTCCAGGCAAGAAT |
| | | CCTGGCTGTGGAAAGATACCTAAAGGATCAACAGCTCCT |
| cPPT/CTS | 71 | TTTTAAAAGAAAAGGGGGGATTGGGGGGTACAGTGCAGG |
| | | GGAAAGAATAGTAGACATAATAGCAACAGACATACAAAC |
| | | TAAAGAATTACAAAAACAAATTACAAAAATTCAAAATTT |
| | | T |
| EF-1-alpha core | 72 | GGGCAGAGCGCACATCGCCCACAGTCCCCGAGAAGTTGG |
| promoter | | GGGGAGGGGTCGGCAATTGAACCGGTGCCTAGAGAAGGT |
| Chimeric Antigen | | GGCGCGGGGTAAACTGGGAAAGTGATGTCGTGTACTGGC |
| | | TCCGCCTTTTTCCCGAGGGTGGGGGAGAACCGTATATAA |
| | | GTGCAGTAGTCGCCGTGAACGTTCTTTTTCGCAACGGGT |
| | | TTGCCGCCAGAACACAG |
| Receptor lacking | 73 | ATGCTCAGGCTGCTCTTGGCTCTCAACTTATTCCCTTCA |
| a VHH domain | | ATTCAAGTAACAGGAGGGTCTTCCTTTTTTGAAGACCCG |
| | | ACGCCAGCGCCAACTATTGCGAGTCAGCCTCTCAGTCTG |
| | | CGACCTGAGGCTTGTCGACCAGCAGCCGGAGGCGCAGTG |
| | | CACACGAGGGGGCTGGACTTCGCCTGTGATCCTTCTAAG |
| | | CCCTTTTGGGTGCTGGTGGTGGTTGGTGGAGTCCTGGCT |
| | | TGCTATAGCTTGCTAGTAACAGTGGCCTTTATTATTTTC |
| | | TGGGTGAGGAAACGGGGCAGAAAGAAACTCCTGTATATA |
| | | TTCAAACAACCATTTATGAGACCAGTACAAACTACTCAA |
| | | GAGGAAGATGGCTGTAGCTGCCGATTTCCAGAAGAAGAA |
| | | GAAGGAGGATGTGAACTGGCTAGCCTGAGAGTGAAGTTC |
| | | AGCAGGAGCGCAGACGCCCCCGCGTACCAGCAGGGCCAG |
| | | AACCAGCTCTATAACGAGCTCAATCTAGGACGAAGAGAG |
| | | GAGTACGATGTTTTGGACAAGAGACGTGGCCGGGACCCT |
| | | GAGATGGGGGGAAAGCCGCAGAGAAGGAAGAACCCTCAG |
| | | GAAGGCCTGTACAATGAACTGCAGAAAGATAAGATGGCG |
| | | GAGGCCTACAGTGAGATTGGGATGAAAGGCGAGCGCCGG |
| | | AGGGGCAAGGGGCACGATGGCCTTTACCAGGGTCTCAGT |
| | | ACAGCCACCAAGGACACCTACGACGCCCTTCACATGCAG |
| | | GCCCTGCCCCCTCGCGCTAGC |
| CD28 signal | 74 | ATGCTCAGGCTGCTCTTGGCTCTCAACTTATTCCCTTCA |
| peptide | | ATTCAAGTAACA |
| CD8a hinge | 75 | CCGACGCCAGCGCCAACTATTGCGAGTCAGCCTCTCAGT |
| region | | CTGCGACCTGAGGCTTGTCGACCAGCAGCCGGAGGCGCA |
| | | GTGCACACGAGGGGGCTGGACTTCGCCTGTGAT |
| CD28 | 76 | CTAAGCCCTTTTGGGTGCTGGTGGTGGTTGGTGGAGTCC |
| transmembrane | | TGGCTTGCTATAGCTTGCTAGTAACAGTGGCCTTTATTA |
| region | | TTTTCTGG |
| 4-1BB | 77 | GTGAGGAAACGGGGCAGAAAGAAACTCCTGTATATATTC |
| costimulation | | AAACAACCATTTATGAGACCAGTACAAACTACTCAAGAG |
| domain | | GAAGATGGCTGTAGCTGCCGATTTCCAGAAGAAGAAGAA |
| | | GGAGGATGTGAACTG |
| CD3z | 78 | CTGAGAGTGAAGTTCAGCAGGAGCGCAGACGCCCCCGCG |
| | | TACCAGCAGGGCCAGAACCAGCTCTATAACGAGCTCAAT |
| | | CTAGGACGAAGAGAGGAGTACGATGTTTTGGACAAGAGA |
| | | CGTGGCCGGGACCCTGAGATGGGGGGAAAGCCGCAGAGA |
| | | AGGAAGAACCCTCAGGAAGGCCTGTACAATGAACTGCAG |
| | | AAAGATAAGATGGCGGAGGCCTACAGTGAGATTGGGATG |
| | | AAAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| P2A | 79 | GCCACGAACTTCTCTCTGTTAAAGCAAGCAGGCGACGTG |
| | | GAAGAAAACCCCGGTCCC |
| eGFP | 80 | GTGAGCAAGGGCGAGGAGCTGTTCACCGGGGTGGTGCCC |
| | | ATCCTGGTCGAGCTGGACGGCGACGTAAACGGCCACAAG |
| | | TTCAGCGTGTCCGGCGAGGGCGAGGGCGATGCCACCTAC |
| | | GGCAAGCTGACCCTGAAGTTCATCTGCACCACCGGCAAG |
| | | CTGCCCGTGCCCTGGCCCACCCTCGTGACCACCCTGACC |
| | | TACGGCGTGCAGTGCTTCAGCCGCTACCCCGACCACATG |
| | | AAGCAGCACGACTTCTTCAAGTCCGCCATGCCCGAAGGC |
| | | TACGTCCAGGAGCGCACCATCTTCTTCAAGGACGACGGC |
| | | AACTACAAGACCCGCGCCGAGGTGAAGTTCGAGGGCGAC |
| | | ACCCTGGTGAACCGCATCGAGCTGAAGGGCATCGACTTC |
| | | AAGGAGGACGGCAACATCCTGGGGCACAAGCTGGAGTAC |
| | | AACTACAACAGCCACAACGTCTATATCATGGCCGACAAG |
| | | CAGAAGAACGGCATCAAGGTGAACTTCAAGATCCGCCAC |
| | | AACATCGAGGACGGCAGCGTGCAGCTCGCCGACCACTAC |
| | | CAGCAGAACACCCCCATCGGCGACGGCCCCGTGCTGCTG |
| | | CCCGACAACCACTACCTGAGCACCCAGTCCGCCCTGAGC |
| | | AAAGACCCCAACGAGAAGCGCGATCACATGGTCCTGCTG |
| | | GAGTTCGTGACCGCCGCCGGGATCACTCTCGGCATGGAC |
| | | GAGCTGTACAAGTAA |
| BbsI site I | | GTCTTC |
| BbsI site II | | GAAGAC |
| WPRE | 81 | AATCAACCTCTGGATTACAAAATTTGTGAAAGATTGACT |
| | | GGTATTCTTAACTATGTTGCTCCTTTTACGCTATGTGGA |
| | | TACGCTGCTTTAATGCCTTTGTATCATGCTATTGCTTCC |
| | | CGTATGGCTTTCATTTTCTCCTCCTTGTATAAATCCTGG |
| | | TTGCTGTCTCTTTATGAGGAGTTGTGGCCCGTTGTCAGG |
| | | CAACGTGGCGTGGTGTGCACTGTGTTTGCTGACGCAACC |
| | | CCCACTGGTTGGGGCATTGCCACCACCTGTCAGCTCCTT |
| | | TCCGGGACTTTCGCTTTCCCCCTCCCTATTGCCACGGCG |
| | | GAACTCATCGCCGCCTGCCTTGCCCGCTGCTGGACAGGG |
| | | GCTCGGCTGTTGGGCACTGACAATTCCGTGGTGTTGTCG |
| | | GGGAAATCATCGTCCTTTCCTTGGCTGCTCGCCTGTGTT |
| | | GCCACCTGGATTCTGCGCGGGACGTCCTTCTGCTACGTC |
| | | CCTTCGGCCCTCAATCCAGCGGACCTTCCTTCCCGCGGC |
| | | CTGCTGCCGGCTCTGCGGCCTCTTCCGCGTCTTTGCCTT |
| | | CGCCCTCAGACGAGTCGGATCTCCCTTTGGGCCGCCTCC |
| | | CCGC |
| Factor Xa site | 82 | TCGGCCCTCAAT |
| 3′ LTR (Delta- | 83 | TGGAAGGGCTAATTCACTCCCAACGAAGATAAGATCTGC |
| U3) | | TTTTTGCTTGTACTGGGTCTCTCTGGTTAGACCAGATCT |
| | | GAGCCTGGGAGCTCTCTGGCTAACTAGGGAACCCACTGC |
| | | TTAAGCCTCAATAAAGCTTGCCTTGAGTGCTTCAAGTAG |
| | | TGTGTGCCCGTCTGTTGTGTGACTCTGGTAACTAGAGAT |
| | | CCCTCAGACCCTTTTAGTCAGTGTGGAAAATCTCTAGCA |
| SV40 poly(A) | 84 | AACTTGTTTATTGCAGCTTATAATGGTTACAAATAAAGC |
| signal | | AATAGCATCACAAATTTCACAAATAAAGCATTTTTTTCA |
| | | CTGCATTCTAGTTGTGGTTTGTCCAAACTCATCAATGTA |
| | | TCTTA |
| MMLV Psi | 85 | AAGCTGGCCAGCAACTTATCTGTGTCTGTCCGATTGTCT |
| | | AGTGTCTATGACTGATTTTATGCGCCTGCGTCGGTACTA |
| | | GTTAGCTAACTAGCTCTGTATCTGGCGGACCCGTGGTGG |
| | | AACTGACGAGTTCGGAACACCCGGCCGCAACCCTGGGAG |
| | | ACGTCCCAGGGACTTCGGGGGCCGTTTTTGTGGCCCGAC |
| | | CTGAGTCCAAAAATCCCGATCGTTTTGGACTCTTTGGTG |
| | | CACCCCCCTTAGAGGAGGGATATGTGGTTCTGGTAGGAG |
| | | ACGAGAACCTAAAACAGTTCCCGCCTCCGTCTGAATTTT |
| | | TGCTTTCGGTTTGGGACCGAAGCCGCGCCGCGCGTCTTG |
| | | TCTGCTG |
| gag (truncated) | 86 | GGCCAGACTGTTACCACTCCCTTAAGTTTGACCTTAGGT |
| | | CACTGGAAAGATGTCGAGCGGATCGCTCACAACCAGTCG |
| | | GTAGATGTCAAGAAGAGACGTTGGGTTACCTTCTGCTCT |
| | | GCAGAATGGCCAACCTTTAACGTCGGATGGCCGCGAGAC |
| | | GGCACCTTTAACCGAGACCTCATCACCCAGGTTAAGATC |
| | | AAGGTCTTTTCACCTGGCCCGCATGGACACCCAGACCAG |
| | | GTCCCCTACATCGTGACCTGGGAAGCCTTGGCTTTTGAC |
| | | CCCCCTCCCTGGGTCAAGCCCTTTGTACACCCTAAGCCT |
| | | CCGCCTCCTCTTCCTCCATCCGCCCCGTCTCTCCCCCTT |
| | | GAACCTCCTCGTTCGACCCCGCCTCGATCCTCCCTTTAT |
| | | CCAGCCCTCACTCCTTCTCTAGGCGCC |
| pol region | 87 | CCATATGAGATCTTATATGGGGCACCCCCGCCCCTTGTA |
| | | AACTTCCCTGACCCTGACATGACAAGAGTTACTAACAGC |
| | | CCCTCTCTCCAAGCTCACTTACAGGCTCTCTACTTAGTC |
| | | CAGCACGAAGTCTGGAGACCTCTGGCGGCAGCCTACCAA |
| | | GAACAACTGGACCGACCGGTGGTACCTCACCCTTACCGA |
| | | GTCGGCGACACAGTGTGGGTCCGCCGACACCAGACTAAG |
| | | AACCTAGAACCTCGCTGGAAAGGACCTTACACAGTCCTG |
| | | CTGACCACCCCCACCGCCCTCAAAGTAGACGGCATCGCA |
| | | GCTTGGATACACGCCGCCCACGTGAAGGCTGCCGACCCC |
| | | GGGGGTGGACCATCCTCTAGACTG |
| NFAT binding | | AGGAAAAA |
| site | | |
| PuroR | 88 | ATGACCGAGTACAAGCCCACGGTGCGCCTCGCCACCCGC |
| | | GACGACGTCCCCAGGGCCGTACGCACCCTCGCCGCCGCG |
| | | TTCGCCGACTACCCCGCCACGCGCCACACCGTCGATCCG |
| | | GACCGCCACATCGAGCGGGTCACCGAGCTGCAAGAACTC |
| | | TTCCTCACGCGCGTCGGGCTCGACATCGGCAAGGTGTGG |
| | | GTCGCGGACGACGGCGCCGCCGTGGCGGTCTGGACCACG |
| | | CCGGAGAGCGTCGAAGCGGGGGCGGTGTTCGCCGAGATC |
| | | GGCCCGCGCATGGCCGAGTTGAGCGGTTCCCGGCTGGCC |
| | | GCGCAGCAACAGATGGAAGGCCTCCTGGCGCCGCACCGG |
| | | CCCAAGGAGCCCGCGTGGTTCCTGGCCACCGTCGGCGTT |
| | | TCGCCCGACCACCAGGGCAAGGGTCTGGGCAGCGCCGTC |
| | | GTGCTCCCCGGAGTGGAGGCGGCCGAGCGCGCCGGGGTG |
| | | CCCGCCTTCCTGGAGACCTCCGCGCCCCGCAACCTCCCC |
| | | TTCTACGAGCGGCTCGGCTTCACCGTCACCGCCGACGTC |
| | | GAGGTGCCCGAAGGACCGCGCACCTGGTGCATGACCCGC |
| | | AAGCCCGGTGCCGCTAGC |
| Furin cleavage | 89 | CGGGCCAAGCGG |
| site | | |
| Linker | 90/ | TCCGGATCCGGA OR TCGGGTTCGGGT |
| | 91 | |
| E2A | 92 | CAGTGCACCAACTATGCGCTGCTGAAACTGGCGGGCGAT |
| | | GTGGAAAGCAACCCGGGCCCG |
| HSV-TK | 93 | ATGGCTTCGTACCCCTGCCATCAACACGCGTCTGCGTTC |
| | | GACCAGGCTGCGCGTTCTCGCGGCCATAGCAACCGACGT |
| | | ACGGCGTTGCGCCCTCGCCGGCAGCAAGAAGCCACGGAA |
| | | GTCCGCCTGGAGCAGAAAATGCCCACGCTACTGCGGGTT |
| | | TATATAGACGGTCCTCACGGGATGGGGAAAACCACCACC |
| | | ACGCAACTGCTGGTGGCCCTGGGTTCGCGCGACGATATC |
| | | GTCTACGTACCCGAGCCGATGACTTACTGGCAGGTGCTG |
| | | GGGGCTTCCGAGACAATCGCGAACATCTACACCACACAA |
| | | CACCGCCTCGACCAGGGTGAGATATCGGCCGGGGACGCG |
| | | GCGGTGGTAATGACAAGCGCCCAGATAACAATGGGCATG |
| | | CCTTATGCCGTGACCGACGCCGTTCTGGCTCCTCATATC |
| | | GGGGGGGAGGCTGGGAGCTCACATGCCCCGCCCCCGGCC |
| | | CTCACCCTCATCTTCGACCGCCATCCCATCGCCGCCCTC |
| | | CTGTGCTACCCGGCCGCGCGATACCTTATGGGCAGCATG |
| | | ACCCCCCAGGCCGTGCTGGCGTTCGTGGCCCTCATCCCG |
| | | CCGACCTTGCCCGGCACAAACATCGTGTTGGGGGCCCTT |
| | | CCGGAGGACAGACACATCGACCGCCTGGCCAAACGCCAG |
| | | CGCCCCGGCGAGCGGCTTGACCTGGCTATGCTGGCCGCG |
| | | ATTCGCCGCGTTTACGGGCTGCTTGCCAATACGGTGCGG |
| | | TATCTGCAGGGCGGCGGGTCGTGGCGGGAGGATTGGGGA |
| | | CAGCTTTCGGGGACGGCCGTGCCGCCCCAGGGTGCCGAG |
| | | CCCCAGAGCAACGCGGGCCCACGACCCCATATCGGGGAC |
| | | ACGTTATTTACCCTGTTTCGGGCCCCCGAGTTGCTGGCC |
| | | CCCAACGGCGACCTGTACAACGTGTTTGCCTGGGCCTTG |
| | | GACGTCTTGGCCAAACGCCTCCGTCCCATGCACGTCTTT |
| | | ATCCTGGATTACGACCAATCGCCCGCCGGCTGCCGGGAC |
| | | GCCCTGCTGCAACTTACCTCCGGGATGGTCCAGACCCAC |
| | | GTCACCACCCCCGGCTCCATACCGACGATCTGCGACCTG |
| | | GCGCGCACGTTTGCCCGGGAGATGGGGGAGGCTAAC |
| mKat | 94 | ATGGTGAGCGAGCTGATTAAGGAGAACATGCACATGAAG |
| | | CTGTACATGGAGGGCACCGTGAACAACCACCACTTCAAG |
| | | TGCACATCCGAGGGCGAAGGCAAGCCCTACGAGGGCACC |
| | | CAGACCATGAGAATCAAGGCGGTCGAGGGCGGCCCTCTC |
| | | CCCTTCGCCTTCGACATCCTGGCTACCAGCTTCATGTAC |
| | | GGCAGCAAAACCTTCATCAACCACACCCAGGGCATCCCC |
| | | GACTTCTTTAAGCAGTCCTTCCCCGAGGGCTTCACATGG |
| | | GAGAGAGTCACCACATACGAAGACGGGGGCGTGCTGACC |
| | | GCTACCCAGGACACCAGCCTCCAGGACGGCTGCCTCATC |
| | | TACAACGTCAAGATCAGAGGGGTGAACTTCCCATCCAAC |
| | | GGCCCTGTGATGCAGAAGAAAACACTCGGCTGGGAGGCC |
| | | TCCACCGAGACCCTGTACCCCGCTGACGGCGGCCTGGAA |
| | | GGCAGAGCCGACATGGCCCTGAAGCTCGTGGGGGGGGGC |
| | | CACCTGATCTGCAACTTGAAGACCACATACAGATCCAAG |
| | | AAACCCGCTAAGAACCTCAAGATGCCCGGCGTCTACTAT |
| | | GTGGACAGAAGACTGGAAAGAATCAAGGAGGCCGACAAA |
| | | GAGACCTACGTCGAGCAGCACGAGGTGGCTGTGGCCAGA |
| | | TACTGCGACCTCCCTAGCAAACTGGGGCACAGATAA |
| Minimal | 95 | TAGAGGGTATATAATGGAAGCTCGATTTCCAG |
| promoter (minP) | | |

TABLE 3B
Amino acid sequences for polypeptides encoded by plasmid features.

| | SEQ ID | |
|---|---|---|
| Feature Name | NO: | Amino Acid Sequence |
| Chimeric Antigen | 96 | MLRLLLALNLFPSIQVTGGSSFFEDPTPAPTIASQPL |
| Receptor lacking a | | SLRPEACRPAAGGAVHTRGLDFACDPSKPFWVLVVVG |
| VHH domain | | GVLACYSLLVTVAFIIFWVRKRGRKKLLYIFKQPFMR |
| | | PVQTTQEEDGCSCRFPEEEEGGCELASLRVKFSRSAD |
| | | APAYQQGQNQLYNELNLGRREEYDVLDKRRGRDPEMG |
| | | GKPQRRKNPQEGLYNELQKDKMAEAYSEIGMKGERRR |
| | | GKGHDGLYQGLSTATKDTYDALHMQALPPRAS |
| CD28 signal peptide | 97 | MLRLLLALNLFPSIQVT |
| CD8a hinge region | 98 | PTPAPTIASQPLSLRPEACRPAAGGAVHTRGLDFACD |
| CD28 | 99 | SKPFWVLVVVGGVLACYSLLVTVAFIIFW |
| transmembrane | | |
| region | | |
| 4-1BB costimulation | 100 | VRKRGRKKLLYIFKQPFMRPVQTTQEEDGCSCRFPEE |
| domain | | EEGGCEL |
| CD3z | 101 | LRVKFSRSADAPAYQQGQNQLYNELNLGRREEYDVLD |
| | | KRRGRDPEMGGKPQRRKNPQEGLYNELQKDKMAEAYS |
| | | EIGMKGERRRGKGHDGLYQGLSTATKDTYDALHMQAL |
| | | PPRAS |
| P2A | 102 | ATNFSLLKQAGDVEENPGP |
| eGFP | 103 | VSKGEELFTGVVPILVELDGDVNGHKFSVSGEGEGDA |
| | | TYGKLTLKFICTTGKLPVPWPTLVTTLTYGVQCFSRY |
| | | PDHMKQHDFFKSAMPEGYVQERTIFFKDDGNYKTRAE |
| | | VKFEGDTLVNRIELKGIDFKEDGNILGHKLEYNYNSH |
| | | NVYIMADKQKNGIKVNFKIRHNIEDGSVQLADHYQQN |
| | | TPIGDGPVLLPDNHYLSTQSALSKDPNEKRDHMVLLE |
| | | FVTAAGITLGMDELYK |
| PuroR | 104 | MTEYKPTVRLATRDDVPRAVRTLAAAFADYPATRHTV |
| | | DPDRHIERVTELQELFLTRVGLDIGKVWVADDGAAVA |
| | | VWTTPESVEAGAVFAEIGPRMAELSGSRLAAQQQMEG |
| | | LLAPHRPKEPAWFLATVGVSPDHQGKGLGSAVVLPGV |
| | | EAAERAGVPAFLETSAPRNLPFYERLGFTVTADVEVP |
| | | EGPRTWCMTRKPGAAS |
| Furin cleavage site | 105 | RAKR |
| Linker | 106 | SGSG |
| E2A | 107 | QCTNYALLKLAGDVESNPGP |
| HSV-TK | 108 | MASYPCHQHASAFDQAARSRGHSNRRTALRPRRQQEA |
| | | TEVRLEQKMPTLLRVYIDGPHGMGKTTTTQLLVALGS |
| | | RDDIVYVPEPMTYWQVLGASETIANIYTTQHRLDQGE |
| | | ISAGDAAVVMTSAQITMGMPYAVTDAVLAPHIGGEAG |
| | | SSHAPPPALTLIFDRHPIAALLCYPAARYLMGSMTPQ |
| | | AVLAFVALIPPTLPGTNIVLGALPEDRHIDRLAKRQR |
| | | PGERLDLAMLAAIRRVYGLLANTVRYLQGGGSWREDW |
| | | GQLSGTAVPPQGAEPQSNAGPRPHIGDTLFTLFRAPE |
| | | LLAPNGDLYNVFAWALDVLAKRLRPMHVFILDYDQSP |
| | | AGCRDALLQLTSGMVQTHVTTPGSIPTICDLARTFAR |
| | | EMGEAN |
| mKat | 109 | MVSELIKENMHMKLYMEGTVNNHHFKCTSEGEGKPYE |
| | | GTQTMRIKAVEGGPLPFAFDILATSFMYGSKTFINHT |
| | | QGIPDFFKQSFPEGFTWERVTTYEDGGVLTATQDTSL |
| | | QDGCLIYNVKIRGVNFPSNGPVMQKKTLGWEASTETL |
| | | YPADGGLEGRADMALKLVGGGHLICNLKTTYRSKKPA |
| | | KNLKMPGVYYVDRRLERIKEADKETYVEQHEVAVARY |
| | | CDLPSKLGHR |

### OTHER EMBODIMENTS

From the foregoing description, it will be apparent that variations and modifications may be made to the various aspects and embodiments described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for identifying a VHH antibody that binds a target antigen, the method comprising:
(a) preparing a library of expression vectors encoding chimeric antigen receptors (CARs), each comprising a VHH domain generated in response to an antigen of interest;
(b) expressing each member of the library of expression vectors in an immortalized immune cell, wherein the immortalized immune cell comprises a selection vector comprising an activation induced promoter operably linked to a resistance gene and a sensitizing gene, wherein the resistance gene provides for positive selection with a positive selection agent and the sensitizing gene provides for negative selection with a negative selection agent;
(c) contacting the immune cell with an antigen that is not the antigen of interest and the negative selection agent; and
(d) contacting the immune cell with the antigen of interest and the positive selection agent, thereby identifying chimeric antigen receptors comprising VHH domains that selectively bind the target antigen.

2. The method of claim 1, wherein the antigen of interest is a polypeptide expressed on the surface of a cell.

3. The method of claim 2, wherein the antigen of interest is associated with a neoplasia.

4. The method of claim 1, wherein the VHH domains were generated in an animal exposed to an immunogenic composition comprising an antigen presenting cell (APC) expressing the antigen of interest.

5. The method of claim 6, wherein the animal belongs to the subfamily Camelinae.

6. The method of claim 1, wherein the expression vectors each comprise a promoter controlling expression of the encoded CAR.

7. The method of claim 1, wherein each CAR comprises from N-terminus to C-terminus, a CD28 signal peptide, a VHH domain, a CD28 transmembrane domain, a CD28 cytoplasmic domain, and a CD3ζ domain.

8. The method of claim 1, wherein the immortalized immune cell is an immune effector cell.

9. The method of claim 1, wherein the activation induced promoter comprises a nuclear factor of activated T cells response element (NFAT RE).

10. The method of claim 9, wherein the activation induced promoter comprises two or more tandem repeats of the NFAT RE.

11. The method of claim 10, wherein the activation induced promoter comprises three tandem repeats of the NFAT RE.

12. The method of claim 9, wherein the activation induced promoter comprises a minimal promoter.

13. The method of claim 1, wherein the selection vector comprises a detectable reporter.

14. The method of claim 1, wherein the negative and positive selection genes encode a single polypeptide comprising a self-cleaving peptide.

15. The method of claim 14, wherein the self-cleaving peptide is P2A or T2A.

16. The method of claim 1, wherein the resistance gene is a puromycin resistance gene and the positive selection agent comprises puromycin.

17. The method of claim 1, wherein the sensitizing gene encodes an HSV thymidine kinase and the negative selection agent comprises ganciclovir.

18. The method of claim 1, further comprising sequencing VHH domains encoded by the immune cells following exposure to the positive selection agent and/or the negative selection agent.

19. A kit suitable for use in the method of claim 1, wherein the kit comprises the immortalized immune cell comprising the selection vector.
